(19) United States
(12) Patent Application Publication
(10) Pub. No.: US 2011/0159011 A1
(43) Pub. Date: Jun. 30, 2011

(54) USES OF IL-22, IL-17, AND IL-1 FAMILY CYTOKINES IN AUTOIMMUNE DISEASES

(75) Inventors: Yijun Carrier, Bedford, MA (US); Hak-Ling Ma, Weymouth, MA (US); Kyriaki Dunussi-Joannopoulos, Belmont, MA (US); Quintus G. Medley, Wellesley, MA (US)

(73) Assignee: Wyeth LLC, Madison, NJ (US)

(21) Appl. No.: 13/060,695

(22) PCT Filed: Aug. 28, 2009
(86) PCT No.: PCT/US2009/055366

§ 371 Date: Feb. 25, 2011

Related U.S. Application Data
(60) Provisional application No. 61/092,743, filed on Aug. 28, 2008.
(60) Provisional application No. 61/193,087, filed on Oct. 27, 2008.

(51) Int. Cl. A61K 39/395; G01N 33/68; C12Q 1/68; A61P 29/00; A61P 19/02; A61P 17/06

(52) U.S. Cl. 424/158.1; 436/86; 436/94; 435/6.12

(57) ABSTRACT

Methods of detecting inflammatory disorders using IL- 1 isoforms are provided. Methods of treating an inflammatory disorder with an anti-IL-1 antibody are also provided. Methods of treating an inflammatory disorder with an anti-IL-1 antibody and at least one of an anti-IL-22 antibody, an anti-IL-17 antibody, or an anti-TNFα antibody are also provided.

This application is related to U.S. Provisional Application No. 61/092,743, filed Aug. 28, 2008 and U.S. Provisional Application No. 61/193,087, filed Oct. 27, 2008, each of which is incorporated herein by reference for any purpose.

### FIELD

Methods of detecting inflammatory disorders using IL-1 isoforms are provided. Methods of treating an inflammatory disorder with an anti-IL-1 antibody are also provided. Methods of treating an inflammatory disorder with an anti-IL-1 antibody and at least one of an anti-IL-22 antibody, an anti-IL-17 antibody, or an anti-TNFα antibody are also provided.

### BACKGROUND

The classical interleukin-1 (IL-1) family cytokines, IL-1α, IL-1β and IL-18, play key roles in inflammation. Several novel members of the IL-1 family of cytokines were identified from DNA database searches for IL-1 homologues. IL-1F6, IL-1F8 and IL-1F9 can be produced by keratinocytes and up-regulated in the inflamed skin. IL-22, a Th17 cell-derived pro-inflammatory cytokine, acts on keratinocytes and induces both antimicrobial peptide and proinflammatory cytokine gene expression.

Interleukin 22 (IL-22) is a member of the Interleukin 10 (IL-10)-like subgroup of type II cytokines. (Renauld, J.-C. Nature Reviews Immunology 3, 667-76 (2003)). The members of this subgroup (i.e., IL-10, IL-19, IL-20, IL-22, IL-24, and IL-26) are proposed to have a conserved six α-helical structural and functional unit that is also shared with the interferons. (Renauld et al. Nature Reviews Immunology 3, 667-76 (2003) and Langer et al. Cytokine & Growth Factor Reviews 15, 33-48 (2004)). IL-22 is produced by activated T helper (Th)17 CD4⁺ lymphocytes, as well as monocytes, and its expression is highly dependent on IL-23 (Liang, S. C. et al. Journal of Experimental Medicine 203, 2271-9 (2006) and Zheng, Y. et al. Nature 445, 648-51 (2007)). IL-22 is known to regulate local tissue inflammation while acting only on non-immune cells, and plays a critical role in mucosal immunity as well as dysregulated inflammation observed in autoimmune disease. (Wolk, K. et al. Immunity 21, 241-54 (2004); Wolk et al., Cytokine & Growth Factor Reviews 17, 367-80 (2006); Wolk et al. Journal of Immunology 168, 5397-402 (2002); Pan et al. Cellular & Molecular Immunology 1, 43-9 (2004); Zenewicz et al. Immunity 27, 647-59 (2007); Aujla, S. J. et al. Nature Medicine 14, 275-81 (2008); and Zheng, Y. et al. Nature Medicine 14, 282-89 (2008)). Recent clinical and pre-clinical studies strongly implicate Th17 cell and IL-22 activities in the progression of psoriasis, a human autoimmune disease of the skin (Zheng et al. Nature 445, 648-51 (2007); Nickoloff et al. Nature Medicine 13, 242-244 (2007); Zaba et al. Journal of Experimental Medicine 204, 3183-94 (2007); Ma et al. Journal of Clinical Investigation in press(2008); Lowes et al. Nature 445, 866-73 (2007); and Wolk et al. European Journal of Immunology 36, 1309-23 (2006). Adminstration of IL-22 has been shown to induce the hyperproliferation of skin keratinocytes and resultant thickening of the epidermis, both characteristics of psoriatic lesions (Boniface et al. Journal of Immunology 174, 3695-702 (2005)). In addition, the administration of IL-22 has been shown to induce gene expression from keratinocytes that appear to be involved in the recruitment of immune cells and the maintenance of psoriatic tissue inflammation (Wolk et al. European Journal of Immunology 36, 1309-23 (2006); Boniface et al. Journal of Immunology 174, 3695-702 (2005); and Sa et al. Journal of Immunology 178, 2229-40 (2007)[erratum appears in J Immunol. 2007 Jun. 1; 178(11): 7487]).

Expression of IL-22 is up-regulated in T cells by IL-9 or ConA (Dumoutier L. et al. (2000) Proc Nail Acad Sci USA 97(18): 10144-9). Further studies have shown that expression of IL-22 mRNA is induced in vivo in response to LPS administration, and that IL-22 modulates parameters indicative of an acute phase response (Dumoutier L. et al. (2000) supra; Pittman D. et al. (2001) Genes and Immunity 2:172). Taken together, these observations indicate that IL-22 plays a critical role in inflammation (Kotenko S. V. (2002) Cytokine & Growth Factor Reviews 13(3): 223-40).

The cell surface receptor for IL-22 is believed to be a receptor complex consisting of an IL-22 receptor (IL-22R) and an IL-22 receptor 2 (IL-10R2) subunit, each of which is a member of the type II cytokine receptor family (CRF2) (Xie M. H. et al. (2000) J Biol Chem 275(40):31335-9; Kotenko S. V. et al. (2001) J Biol Chem 276(4): 2725-32). CRF2 members are receptors for IFNα/β, IFNγ, coagulation factor VIIa, IL-10 and the IL-10 related proteins IL-19, IL-20, IL-22, IL-24, IL-26, as well as the recently identified IFN-like cytokines, IL-28 and IL-29 (Kotenko S. V. (2002) Cytokine & Growth Factor Reviews 13(3):223-40; Kotenko, S. V. et al. (2000) Oncogene 19(21): 2557-65; Sheppard, P. et al. (2003) Nature Immunology 4(1):63-8; Kotenko, S. V. et al. (2003) Nature Immunology 4(1): 69-77). Each of the subunits, or chains, of the IL-22 receptor complex presents on epithelial cells and some fibroblasts within various tissues (Wolk et al. Journal of Immunology 168, 5397-402 (2002); Xie et al. Journal of Biological Chemistry 275, 31335-9 (2000); Kotenko et al. Journal of Biological Chemistry 276, 2725-32 (2001); Ikeuchi et al. Arthritis & Rheumatism 52, 1037-46 (2005); Andoh et al. Gastroenterology 129, 969-84 (2005)). Both chains of the IL-22 receptor complex are also expressed constitutively in a number of organs, and epithelial cell lines derived from these organs have been shown to be responsive to IL-22 in vitro (Kotenko S. V. (2002) Cytokine & Growth Factor Reviews 13(3): 223-40.

While the IL-22R and IL-10R2 subunits individually contribute to the formation of different receptor complexes for other type II cytokines , together the subunits form a single receptor complex that is specific for IL-22. IL-22 is believed to first bind to the extracellular domain (ECD) of IL-22R. (Logsdon et al. Journal of Interferon & Cytokine Research 22, 1099-112 (2002) and Li et al. International Immunopharmacology 4, 693-708 (2004)). Due to a proposed IL-22R-induced conformational change in IL-22, IL-10R2 is able to bind to the IL-22/IL-22R surface (Li et al. International Immunopharmacology 4, 693-708 (2004) and Logsdon et al. Journal of Molecular Biology 342, 503-14 (2004)). The resulting IL-22/IL-22R/IL-10R2 complex, as either a heterotrimer or multimer thereof, transmits a signal into the cell via the JAK/STAT and MAPK (for example, ERK) signaling pathways (Dumoutier et al. Journal of Immunology 164, 1814-9 (2000). Dumoutier et al. Proceedings of the National Academy of Sciences of the United States of America 97, 10144-9 (2000); and Lejeune et al. Journal of Biological Chemistry 277, 33676-82 (2002)). IL-22 induces activation of the JAK/STAT3 and MAPK (for example, ERK) pathways, as well as intermediates of other MAPK pathways (Dumoutier L. et al. (2000) supra; Xie M. H. et al. (2000) supra; Dumoutier L. et al. (2000) J Immunol 164(4): 1814-9; Kotenko S. V. et al. (2001) J Biol Chem 276(4):2725-32; Lejeune, D. et al. (2002) J Biol Chem 277(37):33676-82).

The interaction between IL-22R and IL-10R2 has been characterized in an ELISA-based format using biotinylated cytokine and receptor extracellular domain (ECD) Fc fusion dimers. See, e.g., U.S. Published Patent Application No. 2005-0042220. IL-22 was shown to have measurable affinity for the ECD of IL-22R and no detectable affinity for IL-10R2 alone. IL-22 was also shown to have a substantially greater affinity for IL-22R/IL-10R2 ECD presented as Fc heterodimers. IL-10R2 appears to bind to a surface created by the association between IL-22 and IL-22R, suggesting that IL-10R2 ECD further stabilizes the association of IL-22 within its cytokine receptor complex. See, e.g., U.S. Published Patent Application No. 2005-0042220.

In addition to binding to the IL-22 receptor complex, IL-22 also binds to an IL-22 binding protein (IL-22BP), which is a secreted ‘receptor’ specific for IL-22 and has 33% primary sequence identity to the extracellular domain (ECD) of IL-22R (Dumoutier, L., Lejeune, D., Colau, D. & Renauld, J. C. Cloning and characterization of IL-22 binding protein, a natural antagonist of IL-10-related T cell-derived inducible factor/IL-22. Journal of Immunology 166, 7090-5 (2001)). While a cell surface form of IL-22BP has not been specifically identified, in vitro, IL-22BP has been shown to act as a decoy receptor and block IL-22 signaling into the cell (Dumoutier et al. Journal of Immunology 166, 7090-5 (2001) and Xu et al. Proceedings of the National Academy of Sciences of the United States of America 98, 9511-6 (2001)).

Neutralizing anti-IL-22 antibodies have been generated and characterized in terms of their binding specificity, affinity and IL-22 neutralizing activity. See, e.g., U.S. Published Patent Application No. 2005-0042220. Administration of IL-22 in vivo has been shown to induce parameters of an acute phase response, and the administration of a neutralizing anti-IL-22 antibody has been shown to reduce IL-22 activity and ameliorates inflammatory symptoms in a mouse collagen-induced arthritis (CIA) model See, e.g., U.S. Published Patent Application No. 2005-0042220. In addition, the expression of IL-22 mRNA has been shown to be upregulated within inflamed areas. Accordingly, IL-22 antagonists, such as, e.g., neutralizing anti-IL-22 antibodies and fragments thereof can be used to induce immune suppression in vivo and they provide a promising approach to the treatment of various inflammatory and/or autoimmune disorders.

Th17 cells are defined by their ability to express IL-17A and IL-17F (Aggarwal et al., J. Biol. Chem., (2003) 278:1910-14; Langrish et al., J. Exp. Med., (2005) 201:233-40; Harrington et al., Nat. Immunol., (2005) 6:1123-32; Park et al., Nat. Immunol., (2005) 6:1133-41; Veldhoen et al., Immunity, (2006) 24:179-89; Mangan et al., Nature, (2006) 441:231-34; Bettelli et al., Nature, (2006) 441:235-38). Th17 cell differentiation is initiated by TGF-β signaling in the context of pro-inflammatory cytokines, particularly IL-6, and also IL-1β and TNF-α. Maintenance and survival of Th17 cells, in contrast, are dependent upon IL-23, an IL-12 family member composed of IL-12p40 and IL-23p19 subunits. IL-23 deficient mice produce significantly less IL-17 in several murine disease and infection models (Langrish et al., J. Exp. Med., (2005) 201:233-40; Murphy et al., J. Exp. Med., (2003) 198:1951-57; Happel et al., J. Exp. Med., (2005) 202:761-69; Khader et al., J. Immunol., (2005) 175:788-95). Thus, Th17 differentiation is initiated by TGF-β and pro-inflammatory cytokines and subsequently maintained by IL-23.

The IL-17 family is composed of five family members—IL-17A, IL-17B, IL-17C, IL-17D, IL-17E (IL-25), and IL-17F—that share a relative homology between 17 to 55% (Aggarwal et al., Cytokine Growth Factor Rev., (2003) 14:155-74; Kolls et al., Immunity, (2004) 21:467-76). The expression of IL-17 family members is quite diverse. IL-17A and IL-17F are the most homologous (55%) and are located adjacent to each other on human chromosome 1. IL-17A and IL-17F mRNA are expressed at higher levels in Th17 cells as compared to Th1 or Th2 cells. In contrast, IL-17B, IL-17C, and IL-17D are expressed predominantly in non-lymphoid tissues. IL-17E (IL-25) is expressed in Th2 cells (Fort et al., Immunity, (2001) 15:985-95). In addition to IL-17A and IL-17F, TNF-α, IL-6, and GM-CSF have also been identified as genes induced by IL-23 and potentially expressed by Th17 cells (Langrish et al., J. Exp. Med., (2005) 201:233-40; Infante-Duarte et al., J. Immunol., (2000) 165:6107-15). However, because Th1 cells can express TNF-a and Th2 cells can express IL-6 and GM-CSF, the expression of IL-6, TNF-α, and GM-CSF is not restricted to the Th17 lineage. In contrast, Th17 cells are thought to produce IL-17A and IL-17F in a lineage specific manner.

Subsets of CD4 effector cells are involved in a number of different diseases. In some cases, their activity is helpful to the organism. In other diseases, however, their activity is undesirable or even harmful. Identification of those subsets of cells within the CD4 effector population that are responsible for a particular pathology permits targeted regulation of those cells without unneeded suppression of other CD4 effector cells. Similarly, knowledge of the cytokines produced by cellular subsets and how those cytokines interact is a prerequisite for the development of comprehensive therapies that provide improved management of diseases involving those cytokines

IL-22 is also a Th17 cytokine that can act cooperatively, and in some cases, synergistically, with IL-17A or IL-17F. See U.S. Published Patent Application No. 20080031882. In addition, IL-22 induction by IL-23 has been demonstrated. Id.

### SUMMARY

In certain embodiments, a method of detecting an inflammatory disorder is provided. In certain embodiments, the method of detecting an inflammatory disorder comprises identifying upregulation of at least one of (a) at least one isoform of IL-1 and (b) IL-1Rrp2 in a patient, wherein the at least one isoform of IL-1 is IL-1F6, IL-1F8, or IL-1F9. In certain embodiments, the inflammatory disorder is psoriasis, lupus, or arthritis. In certain embodiments, the upregulation of at least one of (a) the at least one isoform of IL-1 and (b) IL-1Rrp2 is determined by detecting mRNA levels. In certain embodiments, the upregulation of at least one of (a) the at least one isoform of IL-1 and (b) IL-1Rrp2 is determined by detecting protein levels. In certain embodiments, the detection of upregulation of at least two of (a) the at least one isoform of IL-1 and (b) IL-1 Rrp2 is determined by detecting protein levels of at least one of (a) the at least one isoform of IL-1 and (b) IL-1Rrp2 and by detecting mRNA levels of at least one of (a) the at least one isoform of IL-1 and (b) IL-1Rrp2. The expression of the at least one of (a) at least one isoform of IL-1 and (b) IL-1Rrp2 in the patient can be compared to the level of expression in a control sample, where an increase in expression of at least one isoform of IL-1 or IL-1Rrp2 in the patient as compared to expression in the control sample indicates the presence of the inflammatory disorder in the patient.

In certain embodiments, a method of treating an IL-22-associated disorder is provided. In certain embodiments, the method of treating an IL-22-associated disorder comprises administering at least one inhibitor of at least one of IL-1F6, IL-1F8, and IL-1F9 to a patient with said IL-22-associated disorder. In certain embodiments, the at least one inhibitor is an anti-IL-1F6 antibody. In certain embodiments, the at least one inhibitor is an anti-IL-1F8 antibody. In certain embodiments, the at least one inhibitor is an anti-IL-1F9 antibody. In certain embodiments, the at least one inhibitor is an anti-IL-1Rrp2 antibody.

In certain embodiments, a method of treating an IL-1-associated disorder is provided. In certain embodiments, a method of treating an IL-1-associated disorder comprises administering an inhibitor of IL-22 to a patient with said IL-1-associated disorder. In certain embodiments, the inhibitor of IL-22 is an anti-IL-22 antibody.

In certain embodiments, a method of treating an inflammatory disorder is provided. In certain embodiments, a method of treating an inflammatory disorder comprises administering to a patient with an inflammatory disorder a combination of (a) at least one of (i) an anti-IL-1F6 antibody, (ii) an anti-IL-1F8 antibody, (iii) an anti-IL-1F9 antibody, and (iv) an anti-IL-1Rrp2 antibody; and (b) an anti-IL-22 antibody or IL-22 antagonist. In certain embodiments, a method of treating an inflammatory disorder comprises administering to a patient with an inflammatory disorder an anti-IL-1 antibody, such as an anti-IL-1F6 antibody, an anti-IL-1F8 antibody, or an anti-IL-1F9 antibody, and an anti-IL-17A antibody or IL-17A antagonist. In certain embodiments, a method of treating an inflammatory disorder comprises administering to a patient with an inflammatory disorder a combination of (a) at least one of (i) an anti-IL-1F6 antibody, (ii) an anti-IL-1F8 antibody, (iii) an anti-IL-1F9 antibody, and (iv) an anti-IL-1Rrp2 antibody; (b) an anti-IL-22 antibody or 11-22 antagonist; and (c) an anti-IL-17A antibody or IL-17A antagonist. In other embodiments, a method of treating an inflammatory disorder comprises administering to a patient a combination of (a) at least one of (i) an anti-IL-1F6 antibody, (ii) an anti-IL-1F8 antibody, (iii) an anti-IL-1F9 antibody, and (iv) an anti-IL-1Rrp2 antibody; and (b) an anti-TNFα antibody or TNFα antagonist. In certain embodiments, the inflammatory disorder is psoriasis, lupus, or arthritis.

In certain embodiments, a method for determining the effectiveness of a therapeutic agent in the treatment, reduction, prevention, and/or amelioration of an inflammatory disorder in a subject is provided. In certain embodiments, the method for determining the effectiveness of a therapeutic agent comprises detecting the level of gene expression in the subject compared to a level of gene expression in a control sample, wherein the gene expression detected is the gene expression from at least one of IL-1F6, IL-1F8, IL-1F9, IL-1Rrp; and wherein a lower level of gene expression in the subject compared to the control indicates effectiveness of the therapeutic agent in the treatment, reduction, prevention, and/or amelioration of the inflammatory disorder in the subject.

### BRIEF DESCRIPTION OF THE DRAWINGS

FIG. 1 shows the increased expression of IL-1 cytokines IL-1F6, IL-1F8, and IL-1F9 and their receptor IL-1Rrp2 in the psoriatic-like mouse ear tissues. Psoriasis (Pso.) was induced in scid/scid mice with adaptive transfer of wild type CD4⁺ CD25⁻ CD45RB^{hi} T cells while control (Cont.) mice received saline injection. Mouse ears were harvested 70 days after injection. FIGS. 1(a) and (c) show transcript levels of IL-1 cytokines and their receptor IL-1Rrp2 evaluated by quantitative RT-PCR. The Y axis indicates the relative mRNA copies of the indicated gene compared to those of house-keeping gene GAPDH with an assumption of 1,000 copies of GAPDH mRNA per cell. Statistical analysis was done with an un-paired two tail t test. “*” indicates statistic significance (p<0.001). FIG. 1(b) shows IL-1F6 and β-actin proteins in individual ear samples detected by antibodies against the respective proteins (R&D systems) in western blots.

FIG. 2 shows the decreased expression of IL-1 cytokines in the psoriatic-like mouse ear tissues after systemic IL-22 neutralization. CD4⁺ CD25⁻ CD45RB^{hi} T cell recipient mice (n=5) were given 16 mg/kg of IL-22 (IL22-104, Wyeth, filled symbols) or isotype control (open circles) antibody, intraperitoneally once per week for 11 weeks. 48 hours after the last treatment, mouse ears were harvested, transcript copies of the indicated genes were evaluated and shown as relative expression vs. GAPDH. “*” indicates statistic significance (p<0.01).

FIG. 3 shows the increased expression of IL-1 cytokines and their receptor IL-1Rrp2 in mouse ears treated with IL-22. Ears of BALB/c mice (n=4) were injected intradermally every other day for 2 weeks with 500 ng of recombinant mouse IL-22 (BD Biosciences) or saline in a total volume of 20 ul. Six hours after the last treatment, mouse ears were harvested, transcript levels of the indicated genes were evaluated and shown as relative expression vs. GAPDH. “*” indicates statistic significance (p<0.1).

FIG. 4 shows the transcript levels of IL-1F6, IL-1F8, IL-1F9, and receptor IL-1Rrp2 in primary human keratinocytes after treatment with the indicated amount of recombinant human IL-22 for 48 hours. RNA were purified from cell lysates and transcript copies of indicated genes were evaluated and shown as relative expression vs. GAPDH. The data in FIGS. 4(a) and 4(b) represent independent experiments

FIG. 5 shows that IL-22 synergizes with IL-17A to induce IL-1 isoform gene expression in human primary keratinocytes. Cells were harvested 48 hrs after they received no treatment, 200 ng/ml of recombinant human IL-22 (Wyeth) alone, 20 ng/ml of recombinant human IL-17A (Wyeth) alone, or 200 ng/ml of IL-22 and 20 ng/ml of IL-17A. FIGS. 5(a) and 5(c) show the transcript levels of IL-1F6, IL-1F8, and IL-1F9 in the cell lysate. FIG. 5(b) shows protein levels of IL-1F9 and β-actin in cell lysate evaluated by western blot using antibodies against the respective proteins (R&D systems).

FIG. 6 shows the transcript levels of IL-1F6, IL-1F8, IL-1F9, and the receptor IL-1Rrp2 in the paired non-lesional and lesional skin samples of psoriasis patients. RNA was purified from frozen tissue biopsies and gene expression was evaluated by quantitative RT-PCR. FIG. 6(a) shows mean copies of the indicated gene transcript per group ±standard deviation (n=11). Statistical significance was indicated by p values depicted in each plot. FIGS. 6(b) and 6(c) show gene expression in non-lesional and lesional samples from individual patients.

FIG. 7 shows linear gene expression correlations between IL-1F6, IL-1F8, IL-1F9, and Th17 cytokine IL-22 and IL-17A. Transcript copies of IL-1F6, IL-1F8, IL-1F9, and receptor IL-1 IL-1Rrp2 from lesional skin biopsies of human patients were plotted against transcript copies of IL-22 and IL17A in the same tissue samples. FIG. 7(a) shows a positive correlation between the gene expression of IL-1F6, IL-1F8, IL-1F9, and IL-22 or IL-17A in psoriatic skin lesions. R squared and P values are also indicated in each plot. FIG. 7(b) shows no correlation between the gene expression of receptor IL-1Rrp2 and IL-22 or IL-17A in psoriatic skin lesions. R squared and P values are also indicated in each plot.

FIG. 8 is the summary of gene expression correlations between IL-1F6, IL-1F8, and IL-1F9, and other pro-inflammatory bio-markers in human psoriasis skin lesions. Statistical analysis was done using a two-tailed Pearson test with a 95% confidence interval. Correlation was considered significant when p<0.05.

FIG. 9 shows increased IL-1F8 and IL-1F9 gene expression detected in the white blood cells of collagen induced arthritis mice. DBA1 mice were immunized with 200 ng of bovine type II collagen (Chondrex) emulsified in CFA intradermally. On day 21, all mice received a boost of 200 ng of collagen in IFA. On day 35, mice were sacrificed and the blood was collected for gene expression analysis. RNA was purified from white blood cells using QIAGEN RNeasy® blood mini kit (QIAGEN), mRNA levels of IL-1F6, IL-1F8 and IL-1F9 were evaluated with RT-PCR. Relative transcript copies of IL-1F8 and IL-1F9 per group +standard deviation (n=5) were depicted. IL-1F6 mRNA level was under the detection limit. The data in FIG. 9 show one of two independent experiments.

FIG. 10 shows increased IL-1F6 and IL-1F9 gene expression detected in the white blood cells of psoriatic-like mice. Psoriasis was induced in mice as described in FIG. 1. Mouse blood was collected on day 70 after adoptive T cell transfer and subject to gene expression analysis as described for FIG. 9. Relative transcript copies of IL-1F6, IL-1F9 and receptor IL-1Rrp2 per group ±standard deviation (n=5) were depicted. IL-1 F8 mRNA level was under the detection limit. The data in FIG. 10 show one of two independent experiments.

FIG. 11 shows increased receptor IL-1 Rrp2, IL-1F6 and IL-1F9 cytokine gene transcripts detected in the white blood cells of lupus prone NZBWF/1 mice. Blood was collected from 10-week and 7-month old NZBWF/1 strain of mice that are genetically susceptible to spontaneous development of lupus. 10-week old naïve C57BL/6 mice that are not susceptible to spontaneous development of lupus were used as controls. Relative transcript copies of IL-1F6, IL-1F9 and receptor IL-1Rrp2 per group ±standard deviation (n=5) were depicted. IL-1 F8 mRNA level was under the detection limit. The data in FIG. 11 show one of two independent experiments.

FIG. 12 shows the human IL-22 nucleotide sequence and the human IL-22 amino acid sequence.

FIG. 13 shows the mouse IL-22 nucleotide sequence and the mouse IL-22 amino acid sequence.

FIG. 14 shows the human IL-1F6 nucleotide sequence and the human IL-1F6 amino acid sequence.

FIG. 15 shows the human IL-1F8 nucleotide sequence and the human IL-1F8 amino acid sequence.

FIG. 16 shows the human IL-1F9 nucleotide sequence and the human IL-1F9 amino acid sequence.

FIG. 17 shows the human IL-1Rrp2 nucleotide sequence and the human IL-1Rrp2 amino acid sequence.

FIG. 18 shows the human IL-17A mRNA nucleotide sequence and the human IL-17A amino acid sequence.

FIG. 19 shows the fold increase of IL-1F6, IL-1F8, and IL-1F9 expression in keratinocytes 48 hours after incubation with 20 ng/ml of TNF-α and combinations of IL-22 (200 ng/ml) and TNF-α (20 ng/ml). Data from 3 donors were pooled and mean ±SD are depicted.

FIG. 20 shows the fold increase of IL-1F8 and IL-1F9 expression in keratinocytes 48 hours after incubation with indicated concentrations of IL-12 without or with 20 ng/ml of IFN-γ. Fold increase of IL-1 F6 expression was not detected. Data from 5 donors were pooled and mean ±SD are depicted.

FIG. 21 shows the fold increase of IL-1F8 expression in keratinocytes 48 hours after incubation with 20 ng/ml of IL-17A, 20 ng/ml of IFN-γ, or the combination of both. Data from 3 donors were pooled and mean ±SD are depicted.

FIG. 22 shows the expression of IL-1 F8 and IL-1 F9 relative to GAPDH in keratinocytes 48 hours after incubation with 200 ng/ml of IL-21 or a combination of 200 ng/ml of IL-21 and 200 ng/ml of IL-22. Data from 2 individual donors are shown.

FIG. 23 shows the fold increase of illa and illb expression in keratinocytes 48 hours after incubation with IL-22 (200 ng/ml), IL-17A (20 ng/ml), IL-22 (200 ng/ml) plus IL-17A (20 ng/ml), IL-12 (200 ng/ml), IFN-y (20 ng/ml), or IL-12 (200 ng/ml) plus IFN-γ (20 ng/ml). Data from 5 donors were pooled and mean ±SD are depicted.

FIG. 24 shows the fold increase of IL1α (a), IL1β (b) IL-1F6 (c) and IL-1F9 (d) expression in keratinocytes 72 hrs after incubation with 1000 ng/ml of IL-1F6, F8 and F9 or in combination of IL-17A (20 ng/ml), IFN-γ (20 ng/ml) or TNF-α (20 ng/ml). Data from 1 donor are shown.

FIG. 25 shows the fold increase of saa1/2 (a), serpin e1 (b), plau (c), plat (d), tnfa (e) and il6 (f) expression in keratinocytes at 6 hours or 72 hours (G—for il6 only) after incubation with 1000 ng/ml of IL-1F6, IL-1F8 and IL-1F9, alone, or in combination with IL-17A (20 ng/ml), IFN-γ (20 ng/ml) or TNF-α (20 ng/ml). Data from 1 donor are shown.

FIG. 26 shows the expression of s100a7 and def4 relative to GAPDH (a) and (c) or fold increase of s100a7 and def4 gene expression (b) and (d) in keratinocytes 72 hours after incubation with 1000 ng/ml of IL-1F6, IL-1F8 and IL-1F9, alone, or in combination with IL-17A (20 ng/ml), IFN-γ (20 ng/ml) or TNF-α (20 ng/ml). Data from 1 donor are shown.

### DETAILED DESCRIPTION OF CERTAIN EMBODIMENTS

Unless otherwise defined, all technical and scientific terms used herein have the same meaning as commonly understood by one of ordinary skill in the art. Although methods and materials similar or equivalent to those described herein can be used in the practice or testing of the claims, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

In order that the present invention may be more readily understood, certain terms are first defined. Additional definitions are set forth throughout the detailed description. Unless specific definitions are provided, the nomenclatures utilized in connection with, and the laboratory procedures and techniques of, analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, delivery, and treatment of patients.

In this application, the use of the singular includes the plural unless specifically stated otherwise. In this application, the use of “or” means “and/or” unless stated otherwise. In the context of a multiple dependent claim, the use of “or” refers back to more than one preceding independent or dependent claim in the alternative only. Furthermore, the use of the term “including,” as well as other forms, such as “includes” and “included,” is not limiting. Also, terms such as “element” or “component” encompass both elements and components comprising one unit and elements and components that comprise more than one subunit unless specifically stated otherwise.

Other features and advantages will be apparent from the following detailed description and claims.

The present application provides for, at least in part, antibodies and antigen-binding fragments thereof that bind to IL-22, in particular, human IL-22, with high affinity and specificity. In certain embodiments, the anti-IL-22 antibodies or fragments thereof can be used to diagnose, treat or prevent IL-22-associated disorders and/or inflammatory disorders, e.g., autoimmune disorders, e.g., arthritis (including rheumatoid arthritis, juvenile rheumatoid arthritis, osteoarthritis, psoriatic arthritis, lupus-associated arthritis or ankylosing spondylitis), scleroderma, systemic lupus erythematosis, HIV, Sjogren's syndrome, vasculitis, multiple sclerosis, autoimmune thyroiditis, dermatitis (including atopic dermatitis and eczematous dermatitis), myasthenia gravis, inflammatory bowel disease (IBD), Crohn's disease, colitis, diabetes mellitus (type I); inflammatory conditions of, e.g., the skin (e.g., psoriasis), cardiovascular system (e.g., atherosclerosis), nervous system (e.g., Alzheimer's disease), liver (e.g., hepatitis), kidney (e.g., nephritis) and pancreas (e.g., pancreatitis); cardiovascular disorders, e.g., cholesterol metabolic disorders, oxygen free radical injury, ischemia; disorders associated with wound healing; respiratory disorders, e.g., asthma and COPD (e.g., cystic fibrosis); acute inflammatory conditions (e.g., endotoxemia, sepsis and septicaemia, toxic shock syndrome and infectious disease); transplant rejection and allergy. In one embodiment, the IL-22-associated disorder is, an arthritic disorder, e.g., a disorder chosen from one or more of rheumatoid arthritis, juvenile rheumatoid arthritis, osteoarthritis, psoriatic arthritis, or ankylosing spondylitis; a respiratory disorder (e.g., asthma, chronic obstructive pulmonary disease (COPD); or an inflammatory condition of, e.g., the skin (e.g., psoriasis), cardiovascular system (e.g., atherosclerosis), nervous system (e.g., Alzheimer's disease), liver (e.g., hepatitis), kidney (e.g., nephritis), pancreas (e.g., pancreatitis), and gastrointestinal organs, e.g., colitis, Crohn's disease and IBD.

The term “interleukin-22” or “IL-22” refers to a class II cytokine (which may be mammalian) capable of binding to IL-22R and/or a receptor complex of IL-22R and IL-10R2, and has at least one of the following features: (1) an amino acid sequence of a naturally occurring mammalian IL-22 polypeptide (full length or mature form) or a fragment thereof, e.g., an amino acid sequence shown as SEQ ID NO:1 (human) or SEQ ID NO:3 (murine) or a fragment thereof; (2) an amino acid sequence substantially identical to, e.g., at least 85%, 90%, 91%, 92%, 93%, 94%, 95%, 96%, 97%, 98%, or 99% identical to, an amino acid sequence shown as SEQ ID NO:1 or amino acids 34-179 thereof (human) or SEQ ID NO:3 (murine) or a fragment thereof; (3) an amino acid sequence which is encoded by a naturally occurring mammalian IL-22 nucleotide sequence or a fragment thereof (e.g., SEQ ID NO:2 or nucleotides 71 to 610 (human) or SEQ ID NO:4 (murine) or a fragment thereof); (4) an amino acid sequence encoded by a nucleotide sequence which is substantially identical to, e.g., at least 85%, 90%, 91%, 92%, 93%, 94%, 95%, 96%, 97%, 98%, or 99% identical to, a nucleotide sequence shown as SEQ ID NO:2 or nucleotides 71 to 610 thereof (human) or SEQ ID NO:4 (murine) or a fragment thereof; (5) an amino acid sequence encoded by a nucleotide sequence degenerate to a naturally occurring IL-22 nucleotide sequence or a fragment thereof, e.g., SEQ ID NO:2 (human) or SEQ ID NO:4 (murine) or a fragment thereof; or (6) a nucleotide sequence that hybridizes to one of the foregoing nucleotide sequences under stringent conditions, e.g., highly stringent conditions. The IL-22 may bind to IL-22R and/or a receptor complex of IL-22R and IL-10R2 of mammalian origin, e.g., human or mouse.

The human IL-22 cDNA was deposited with the American Type Culture Collection (10801 University Boulevard, Manassas, Va., U.S.A. 20110-2209) on Apr. 28, 1999 as an original deposit under the Budapest Treaty and was given the accession number ATCC 207231.

The phrase “an IL-22 activity” or “IL-22 associated activity” refers to one or more of the biological activities of an IL-22 polypeptide, e.g., a mature IL-22 polypeptide (e.g., a mammalian, e.g., human or murine IL-22 having an amino acid sequence as shown in SEQ ID NO:2 and 4, respectively), including, but not limited to, (1) interacting with, e.g., binding to, an IL-22 receptor (e.g., an IL-22R or IL-10R2 or a complex thereof, preferably of mammalian, e.g., murine or human origin); (2) associating with one or more signal transduction molecules; (3) stimulating phosphorylation and/or activation of a protein kinase, e.g., JAK/STAT3, ERK, and MAPK; (4) modulating, e.g., stimulating or decreasing, proliferation, differentiation, effector cell function, cytolytic activity, cytokine or chemokine secretion, and/or survival of an IL-22 responsive cell, e.g., an epithelial cell from, e.g., kidney, liver, colon, small intestine, thyroid gland, pancreas, skin); (5) modulating at least one parameter of an acute phase response, e.g., a metabolic, hepatic, hematopoietic (e.g., anemia, platelet increase) or neuroendocrine change, or a change (e.g., increase or decrease in an acute phase protein, e.g., an increase in fibrinogen and/or serum amyloid A, or a decrease in albumin); and/or (6) modulating at least one parameter of an inflammatory state, e.g., modulating cytokine-mediated proinflammatory actions (e.g., fever, and/or prostaglandin synthesis, for example PGE₂ synthesis), modulating cellular immune responses, modulating cytokine, chemokine (e.g., GRO1), or lymphokine production and/or secretion (e.g., production and/or secretion of a proinflammatory cytokine)

The present application provides for, at least in part, antibodies and antigen-binding fragments thereof that bind to IL-1F6, in particular, human IL-1F6, with high affinity and specificity. In certain embodiments, the anti-IL-1F6 antibodies or fragments thereof can be used to diagnose, treat or prevent IL-1F6-associated disorders and/or inflammatory disorders, e.g., autoimmune disorders, e.g., arthritis (including rheumatoid arthritis, juvenile rheumatoid arthritis, osteoarthritis, psoriatic arthritis, lupus-associated arthritis or ankylosing spondylitis), scleroderma, systemic lupus erythematosis, HIV, Sjogren's syndrome, vasculitis, multiple sclerosis, autoimmune thyroiditis, dermatitis (including atopic dermatitis and eczematous dermatitis), myasthenia gravis, inflammatory bowel disease (IBD), Crohn's disease, colitis, diabetes mellitus (type I); inflammatory conditions of, e.g., the skin (e.g., psoriasis), cardiovascular system (e.g., atherosclerosis), nervous system (e.g., Alzheimer's disease), liver (e.g., hepatitis), kidney (e.g., nephritis) and pancreas (e.g., pancreatitis); cardiovascular disorders, e.g., cholesterol metabolic disorders, oxygen free radical injury, ischemia; disorders associated with wound healing; respiratory disorders, e.g., asthma and COPD (e.g., cystic fibrosis); acute inflammatory conditions (e.g., endotoxemia, sepsis and septicaemia, toxic shock syndrome and infectious disease); transplant rejection and allergy. In one embodiment, the IL-1F6-associated disorder is, an arthritic disorder, e.g., a disorder chosen from one or more of rheumatoid arthritis, juvenile rheumatoid arthritis, osteoarthritis, psoriatic arthritis, or ankylosing spondylitis; a respiratory disorder (e.g., asthma, chronic obstructive pulmonary disease (COPD); or an inflammatory condition of, e.g., the skin (e.g., psoriasis), cardiovascular system (e.g., atherosclerosis), nervous system (e.g., Alzheimer's disease), liver (e.g., hepatitis), kidney (e.g., nephritis), pancreas (e.g., pancreatitis), and gastrointestinal organs, e.g., colitis, Crohn's disease and IBD.

The term “IL-1F6” refers to an IL-1 cytokine, and has at least one of the following features: (1) an amino acid sequence of a naturally occurring mammalian IL-F6 polypeptide (full length or mature form) or a fragment thereof, e.g., an amino acid sequence shown as SEQ ID NO:6 or a fragment thereof; (2) an amino acid sequence substantially identical to, e.g., at least 85%, 90%, 91%, 92%, 93%, 94%, 95%, 96%, 97%, 98%, or 99% identical to, an amino acid sequence shown as SEQ ID NO:6 or a fragment thereof; (3) an amino acid sequence which is encoded by a naturally occurring mammalian IL-1F6 nucleotide sequence or a fragment thereof (e.g., SEQ ID NO:5 or a fragment thereof); (4) an amino acid sequence encoded by a nucleotide sequence which is substantially identical to, e.g., at least 85%, 90%, 91%, 92%, 93%, 94%, 95%, 96%, 97%, 98%, or 99% identical to, a nucleotide sequence shown as SEQ ID NO:5 or a fragment thereof; (5) an amino acid sequence encoded by a nucleotide sequence degenerate to a naturally occurring IL-1F6 nucleotide sequence or a fragment thereof, e.g., SEQ ID NO:5 or a fragment thereof; or (6) a nucleotide sequence that hybridizes to SEQ ID NO:5 under stringent conditions, e.g., highly stringent conditions.

The present application provides for, at least in part, antibodies and antigen-binding fragments thereof that bind to IL-1F8, in particular, human IL-1F8, with high affinity and specificity. In certain embodiments, the anti-IL-1F8 antibodies or fragments thereof can be used to diagnose, treat or prevent IL-1F8-associated disorders and/or inflammatory disorders, e.g., autoimmune disorders, e.g., arthritis (including rheumatoid arthritis, juvenile rheumatoid arthritis, osteoarthritis, psoriatic arthritis, lupus-associated arthritis or ankylosing spondylitis), scleroderma, systemic lupus erythematosis, HIV, Sjogren's syndrome, vasculitis, multiple sclerosis, autoimmune thyroiditis, dermatitis (including atopic dermatitis and eczematous dermatitis), myasthenia gravis, inflammatory bowel disease (IBD), Crohn's disease, colitis, diabetes mellitus (type I); inflammatory conditions of, e.g., the skin (e.g., psoriasis), cardiovascular system (e.g., atherosclerosis), nervous system (e.g., Alzheimer's disease), liver (e.g., hepatitis), kidney (e.g., nephritis) and pancreas (e.g., pancreatitis); cardiovascular disorders, e.g., cholesterol metabolic disorders, oxygen free radical injury, ischemia; disorders associated with wound healing; respiratory disorders, e.g., asthma and COPD (e.g., cystic fibrosis); acute inflammatory conditions (e.g., endotoxemia, sepsis and septicaemia, toxic shock syndrome and infectious disease); transplant rejection and allergy. In one embodiment, the IL-1F8-associated disorder is, an arthritic disorder, e.g., a disorder chosen from one or more of rheumatoid arthritis, juvenile rheumatoid arthritis, osteoarthritis, psoriatic arthritis, or ankylosing spondylitis; a respiratory disorder (e.g., asthma, chronic obstructive pulmonary disease (COPD); or an inflammatory condition of, e.g., the skin (e.g., psoriasis), cardiovascular system (e.g., atherosclerosis), nervous system (e.g., Alzheimer's disease), liver (e.g., hepatitis), kidney (e.g., nephritis), pancreas (e.g., pancreatitis), and gastrointestinal organs, e.g., colitis, Crohn's disease and IBD.

The term “IL-1F8” refers to an IL-1 cytokine, and has at least one of the following features: (1) an amino acid sequence of a naturally occurring mammalian IL-F8 polypeptide (full length or mature form) or a fragment thereof, e.g., an amino acid sequence shown as SEQ ID NO:8 or a fragment thereof; (2) an amino acid sequence substantially identical to, e.g., at least 85%, 90%, 91%, 92%, 93%, 94%, 95%, 96%, 97%, 98%, or 99% identical to, an amino acid sequence shown as SEQ ID NO:8 or a fragment thereof; (3) an amino acid sequence which is encoded by a naturally occurring mammalian IL-1F8 nucleotide sequence or a fragment thereof (e.g., SEQ ID NO:7 or a fragment thereof); (4) an amino acid sequence encoded by a nucleotide sequence which is substantially identical to, e.g., at least 85%, 90%, 91%, 92%, 93%, 94%, 95%, 96%, 97%, 98%, or 99% identical to, a nucleotide sequence shown as SEQ ID NO:7 or a fragment thereof; (5) an amino acid sequence encoded by a nucleotide sequence degenerate to a naturally occurring IL-1F8 nucleotide sequence or a fragment thereof, e.g., SEQ ID NO:7 or a fragment thereof; or (6) a nucleotide sequence that hybridizes to SEQ ID NO:7 under stringent conditions, e.g., highly stringent conditions.

The present application provides for, at least in part, antibodies and antigen-binding fragments thereof that bind to IL-1F9, in particular, human IL-1F9, with high affinity and specificity. In certain embodiments, the anti-IL-1F9 antibodies or fragments thereof can be used to diagnose, treat or prevent IL-1F9-associated disorders and/or inflammatory disorders, e.g., autoimmune disorders, e.g., arthritis (including rheumatoid arthritis, juvenile rheumatoid arthritis, osteoarthritis, psoriatic arthritis, lupus-associated arthritis or ankylosing spondylitis), scleroderma, systemic lupus erythematosis, HIV, Sjogren's syndrome, vasculitis, multiple sclerosis, autoimmune thyroiditis, dermatitis (including atopic dermatitis and eczematous dermatitis), myasthenia gravis, inflammatory bowel disease (IBD), Crohn's disease, colitis, diabetes mellitus (type I); inflammatory conditions of, e.g., the skin (e.g., psoriasis), cardiovascular system (e.g., atherosclerosis), nervous system (e.g., Alzheimer's disease), liver (e.g., hepatitis), kidney (e.g., nephritis) and pancreas (e.g., pancreatitis); cardiovascular disorders, e.g., cholesterol metabolic disorders, oxygen free radical injury, ischemia; disorders associated with wound healing; respiratory disorders, e.g., asthma and COPD (e.g., cystic fibrosis); acute inflammatory conditions (e.g., endotoxemia, sepsis and septicaemia, toxic shock syndrome and infectious disease); transplant rejection and allergy. In one embodiment, the IL-1F9-associated disorder is, an arthritic disorder, e.g., a disorder chosen from one or more of rheumatoid arthritis, juvenile rheumatoid arthritis, osteoarthritis, psoriatic arthritis, or ankylosing spondylitis; a respiratory disorder (e.g., asthma, chronic obstructive pulmonary disease (COPD); or an inflammatory condition of, e.g., the skin (e.g., psoriasis), cardiovascular system (e.g., atherosclerosis), nervous system (e.g., Alzheimer's disease), liver (e.g., hepatitis), kidney (e.g., nephritis), pancreas (e.g., pancreatitis), and gastrointestinal organs, e.g., colitis, Crohn's disease and IBD.

The term “IL-1F9” refers to an IL-1 cytokine, and has at least one of the following features: (1) an amino acid sequence of a naturally occurring mammalian IL-F9 polypeptide (full length or mature form) or a fragment thereof, e.g., an amino acid sequence shown as SEQ ID NO:10 or a fragment thereof; (2) an amino acid sequence substantially identical to, e.g., at least 85%, 90%, 91%, 92%, 93%, 94%, 95%, 96%, 97%, 98%, or 99% identical to, an amino acid sequence shown as SEQ ID NO:10 or a fragment thereof; (3) an amino acid sequence which is encoded by a naturally occurring mammalian IL-1F9 nucleotide sequence or a fragment thereof (e.g., SEQ ID NO:9 or a fragment thereof); (4) an amino acid sequence encoded by a nucleotide sequence which is substantially identical to, e.g., at least 85%, 90%, 91%, 92%, 93%, 94%, 95%, 96%, 97%, 98%, or 99% identical to, a nucleotide sequence shown as SEQ ID NO:9 or a fragment thereof; (5) an amino acid sequence encoded by a nucleotide sequence degenerate to a naturally occurring IL-1F9 nucleotide sequence or a fragment thereof, e.g., SEQ ID NO:9 or a fragment thereof; or (6) a nucleotide sequence that hybridizes to SEQ ID NO:9 under stringent conditions, e.g., highly stringent conditions.

The present application provides for, at least in part, antibodies and antigen-binding fragments thereof that bind to IL-1Rrp2, in particular, human IL-1Rrp2, with high affinity and specificity. In certain embodiments, the anti-IL-1Rrp2 antibodies or fragments thereof can be used to diagnose, treat or prevent IL-1Rrp2-associated disorders and/or inflammatory disorders, e.g., autoimmune disorders, e.g., arthritis (including rheumatoid arthritis, juvenile rheumatoid arthritis, osteoarthritis, psoriatic arthritis, lupus-associated arthritis or ankylosing spondylitis), scleroderma, systemic lupus erythematosis, HIV, Sjogren's syndrome, vasculitis, multiple sclerosis, autoimmune thyroiditis, dermatitis (including atopic dermatitis and eczematous dermatitis), myasthenia gravis, inflammatory bowel disease (IBD), Crohn's disease, colitis, diabetes mellitus (type I); inflammatory conditions of, e.g., the skin (e.g., psoriasis), cardiovascular system (e.g., atherosclerosis), nervous system (e.g., Alzheimer's disease), liver (e.g., hepatitis), kidney (e.g., nephritis) and pancreas (e.g., pancreatitis); cardiovascular disorders, e.g., cholesterol metabolic disorders, oxygen free radical injury, ischemia; disorders associated with wound healing; respiratory disorders, e.g., asthma and COPD (e.g., cystic fibrosis); acute inflammatory conditions (e.g., endotoxemia, sepsis and septicaemia, toxic shock syndrome and infectious disease); transplant rejection and allergy. In one embodiment, the IL-1Rrp2-associated disorder is, an arthritic disorder, e.g., a disorder chosen from one or more of rheumatoid arthritis, juvenile rheumatoid arthritis, osteoarthritis, psoriatic arthritis, or ankylosing spondylitis; a respiratory disorder (e.g., asthma, chronic obstructive pulmonary disease (COPD); or an inflammatory condition of, e.g., the skin (e.g., psoriasis), cardiovascular system (e.g., atherosclerosis), nervous system (e.g., Alzheimer's disease), liver (e.g., hepatitis), kidney (e.g., nephritis), pancreas (e.g., pancreatitis), and gastrointestinal organs, e.g., colitis, Crohn's disease and IBD.

The term “IL-1 Rrp2” refers to an IL-1 cytokine receptor (interleukin 1 receptor-like 2 (IL-1RL2)), and has at least one of the following features: (1) an amino acid sequence of a naturally occurring mammalian IL-Rrp2 polypeptide (full length or mature form) or a fragment thereof, e.g., an amino acid sequence shown as SEQ ID NO:12 or a fragment thereof; (2) an amino acid sequence substantially identical to, e.g., at least 85%, 90%, 91%, 92%, 93%, 94%, 95%, 96%, 97%, 98%, or 99% identical to, an amino acid sequence shown as SEQ ID NO:12 or a fragment thereof; (3) an amino acid sequence which is encoded by a naturally occurring mammalian IL-1Rrp2 nucleotide sequence or a fragment thereof (e.g., SEQ ID NO:11 or a fragment thereof); (4) an amino acid sequence encoded by a nucleotide sequence which is substantially identical to, e.g., at least 85%, 90%, 91%, 92%, 93%, 94%, 95%, 96%, 97%, 98%, or 99% identical to, a nucleotide sequence shown as SEQ ID NO:11 or a fragment thereof; (5) an amino acid sequence encoded by a nucleotide sequence degenerate to a naturally occurring IL-1 Rrp2 nucleotide sequence or a fragment thereof, e.g., SEQ ID NO:11 or a fragment thereof; or (6) a nucleotide sequence that hybridizes to SEQ ID NO:11 under stringent conditions, e.g., highly stringent conditions.

The present application provides for, at least in part, antibodies and antigen-binding fragments thereof that bind to IL-17A, in particular, human IL-17A, with high affinity and specificity. In certain embodiments, the anti-IL-17A antibodies or fragments thereof can be used to diagnose, treat or prevent IL-17A-associated disorders and/or inflammatory disorders, e.g., autoimmune disorders, e.g., arthritis (including rheumatoid arthritis, juvenile rheumatoid arthritis, osteoarthritis, psoriatic arthritis, lupus-associated arthritis or ankylosing spondylitis), scleroderma, systemic lupus erythematosis, HIV, Sjogren's syndrome, vasculitis, multiple sclerosis, autoimmune thyroiditis, dermatitis (including atopic dermatitis and eczematous dermatitis), myasthenia gravis, inflammatory bowel disease (IBD), Crohn's disease, colitis, diabetes mellitus (type I); inflammatory conditions of, e.g., the skin (e.g., psoriasis), cardiovascular system (e.g., atherosclerosis), nervous system (e.g., Alzheimer's disease), liver (e.g., hepatitis), kidney (e.g., nephritis) and pancreas (e.g., pancreatitis); cardiovascular disorders, e.g., cholesterol metabolic disorders, oxygen free radical injury, ischemia; disorders associated with wound healing; respiratory disorders, e.g., asthma and COPD (e.g., cystic fibrosis); acute inflammatory conditions (e.g., endotoxemia, sepsis and septicaemia, toxic shock syndrome and infectious disease); transplant rejection and allergy. In one embodiment, the IL-17A-associated disorder is, an arthritic disorder, e.g., a disorder chosen from one or more of rheumatoid arthritis, juvenile rheumatoid arthritis, osteoarthritis, psoriatic arthritis, or ankylosing spondylitis; a respiratory disorder (e.g., asthma, chronic obstructive pulmonary disease (COPD); or an inflammatory condition of, e.g., the skin (e.g., psoriasis), cardiovascular system (e.g., atherosclerosis), nervous system (e.g., Alzheimer's disease), liver (e.g., hepatitis), kidney (e.g., nephritis), pancreas (e.g., pancreatitis), and gastrointestinal organs, e.g., colitis, Crohn's disease and IBD.

The term “IL-17A” refers to an IL-17 cytokine, and has at least one of the following features: (1) an amino acid sequence of a naturally occurring mammalian IL-17A polypeptide (full length or mature form) or a fragment thereof, e.g., an amino acid sequence shown as SEQ ID NO:14 or a fragment thereof; (2) an amino acid sequence substantially identical to, e.g., at least 85%, 90%, 91%, 92%, 93%, 94%, 95%, 96%, 97%, 98%, or 99% identical to, an amino acid sequence shown as SEQ ID NO:14 or a fragment thereof; (3) an amino acid sequence which is encoded by a naturally occurring mammalian IL-17A nucleotide sequence or a fragment thereof (e.g., SEQ ID NO:13 or a fragment thereof); (4) an amino acid sequence encoded by a nucleotide sequence which is substantially identical to, e.g., at least 85%, 90%, 91%, 92%, 93%, 94%, 95%, 96%, 97%, 98%, or 99% identical to, a nucleotide sequence shown as SEQ ID NO:13 or a fragment thereof; (5) an amino acid sequence encoded by a nucleotide sequence degenerate to a naturally occurring IL-17A nucleotide sequence or a fragment thereof, e.g., SEQ ID NO:13 or a fragment thereof; or (6) a nucleotide sequence that hybridizes to SEQ ID NO:13 under stringent conditions, e.g., highly stringent conditions.

The term “cytokine activity”, whether used generically or as applied to a particular cytokines such as, but not limited to, IL-22, IL-17A, IL-1F6, IL-1F8, or IL-1F9, refers to at least one cellular process initiated or interrupted as a result of binding of that cytokine to its receptor(s) on a cell. Cytokine activities for IL-22 include at least one of, but are not limited to: (1) binding to a cellular receptor subunit or complex, such as IL-22R1, IL-10R2, or IL-22R1/IL-10R2; (2) associating with signal transduction molecules (e.g., JAK-1); (3) stimulating phosphorylation of STAT proteins (e.g., STATS, STAT3, or combination thereof); (4) activating STAT proteins; (5) inducing parameters indicative of an acute phase response, including the modulation of acute phase reactants (e.g., serum amyloid A, fibrinogen, haptoglobin, or serum albumin) and cells (e.g., neutrophils, platelets, or red blood cells; and (6) modulating (e.g., increasing or decreasing) proliferation, differentiation, effector cell function, cytolytic activity, cytokine secretion, survival, or combinations thereof, of epithelial cells, fibroblasts, or immune cells. Epithelial cells include, but are not limited to, cells of the skin, gut, liver, and kidney, as well as endothelial cells. Fibroblasts include, but are not limited to, synovial fibroblasts. Immune cells may include CD8⁺ and CD4⁺ T cells, NK cells, B cells, macrophages, megakaryocytes, and specialized or tissue immune cells, such as those found in inflammed tissues or those expressing an IL-22 receptor.

Cytokine activities for IL-17A and IL-17F include at least one of, but are not limited to: (1) binding to a cellular receptor, such as IL-17R, IL-17A, IL-17RC, IL-17RH1, IL-17RL, IL-17RD, or IL-17RE ; (2) inhibition of angiogenesis; (3) modulating (e.g., increasing or decreasing) proliferation, differentiation, effector cell function, cytolytic activity, cytokine secretion, survival, or combinations thereof, of hematopoietic cells or cells present in cartilage, bone, meniscus, brain, kidney, lung, skin and intestine; (4) inducing production of IL-6 and/or IL-8; and (5) stimulating nitric oxide production.

The terms “induce”, “reduce,” “inhibit,” “potentiate,” “elevate,” “increase,” “decrease” or the like, e.g., which denote quantitative differences between two states, refer to at least statistically significant differences between the two states.

The terms “specific binding” or “specifically binds” refers to two molecules forming a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the association constant K_{A} is higher than 10⁶M⁻¹. If necessary, nonspecific binding can be reduced without substantially affecting specific binding by varying the binding conditions. The appropriate binding conditions, such as concentration of antibodies, ionic strength of the solution, temperature, time allowed for binding, concentration of a blocking agent (e.g., serum albumin, milk casein), etc., may be optimized by a skilled artisan using routine techniques.

The term “specific binding agent” refers to a natural or non-natural molecule that specifically binds to a target. Examples of specific binding agents include, but are not limited to, proteins, peptides, nucleic acids, carbohydrates, lipids, and small molecule compounds. In certain embodiments, a specific binding agent is an antibody. In certain embodiments, a specific binding agent is an antigen binding region.

The term “structure” encompasses both structures of biologics (for example and not limitation, antibodies and fragments thereof) and small molecules.

The term “antibody” refers to an immunoglobulin or fragment thereof, such as Fab, Fab′, F(ab′)₂, Fc, Fd, Fd′, Fv, single chain antibodies (scFv for example), single variable domain antibodies (Dab), diabodies (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The antibodies of the present invention can be monoclonal or polyclonal. The antibody can also be a monospecific, polyspecific, non-specific, humanized, human, single-chain, chimeric, synthetic, recombinant, hybrid, mutated, CDR-grafted, and/or in vitro generated antibody. The antibody can have a heavy chain constant region chosen from, e.g., IgG1, IgG2, IgG3, or IgG4. The antibody can also have a light chain chosen from, e.g., kappa or lambda. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function). Typically, the antibody specifically binds to a predetermined antigen, e.g., an antigen associated with a disorder, e.g., a neurodegenerative, metabolic, inflammatory, autoimmune and/or a malignant disorder. Unless preceded by the word “intact”, the term “antibody” includes, in addition to complete antibody molecules, antibody fragments such as Fab, F(ab′)₂, Fv, scFv, Fd, dAb, and other antibody fragments that retain antigen-binding function. Typically, such fragments comprise an antigen-binding domain.

The antibody can further include a heavy and light chain constant region, to thereby form a heavy and light immunoglobulin chain, respectively. In certain embodiments, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

The terms “antigen-binding domain” and “antigen-binding fragment” refer to a part of an antibody molecule that comprises amino acids responsible for the specific binding between antibody and antigen. The part of the antigen that is specifically recognized and bound by the antibody is referred to as the “epitope.” An antigen-binding domain may comprise an antibody light chain variable region (V_{L}) and an antibody heavy chain variable region (V_{H}); however, it does not have to comprise both. Fd fragments, for example, have two V_{H} regions and often retain some antigen-binding function of the intact antigen-binding domain. Examples of antigen-binding fragments of an antibody include (1) a Fab fragment, a monovalent fragment having the V_{L}, V_{H}, C_{L} and C_{H}1 domains; (2) a F(ab′)₂ fragment, a bivalent fragment having two Fab fragments linked by a disulfide bridge at the hinge region; (3) a Fd fragment having the two V_{H} and C_{H}1 domains; (4) a Fv fragment having the V_{L} and V_{H} domains of a single arm of an antibody, (5) a dAb fragment (Ward et al., (1989) Nature 341:544-546), which has a V_{H} domain; (6) an isolated complementarity determining region (CDR); and (7) a single chain Fv (scFv). Although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). These antibody fragments are obtained using conventional techniques known to those with skill in the art, and the fragments are evaluated for function in the same manner as are intact antibodies.

As used herein, the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd or 214 amino acids) are encoded by a variable region gene at the NH2-terminus (about 110 amino acids) and a kappa or lambda constant region gene at the COOH-terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids).

As used herein, “isotype” refers to the antibody class (e.g., IgM or IgGI) that is encoded by heavy chain constant region genes.

The term “human antibody” includes antibodies having variable and constant regions corresponding substantially to human germline immunoglobulin sequences known in the art, including, for example, those described by Kabat et al. (See Kabat, et al. (1991) Sequences of Proteins of Immunological Interest, Fifth Edition, U.S. Department of Health and Human Services, NIH Publication No. 91-3242). In certain embodiments, human antibodies may include amino acid residues not encoded by human germline immunoglobulin sequences (e.g., mutations introduced by random or site-specific mutagenesis in vitro or by somatic mutation in vivo), for example in the CDRs, and in particular, CDR3. The human antibody can have at least one, two, three, four, five, or more positions replaced with an amino acid residue that is not encoded by the human germline immunoglobulin sequence.

The term “isolated” refers to a molecule that is substantially free of its natural environment. For instance, an isolated protein is substantially free of cellular material or other proteins from the cell or tissue source from which it was derived. The term also refers to preparations where the isolated protein is sufficiently pure for pharmaceutical compositions; or at least 70-80% (w/w) pure; or at least 80-90% (w/w) pure; or at least 90-95% pure; or at least 95%, 96%, 97%, 98%, 99%, or 100% (w/w) pure.

The term “therapeutic agent” is a substance that treats or assists in treating a medical disorder. Therapeutic agents may include, but are not limited to, substances that modulate immune cells or immune responses in a manner that complements the IL-22 activity of anti-IL-22 antibodies. Non-limiting examples and uses of therapeutic agents are described herein.

The term “treatment” refers to a therapeutic or preventative measure. The treatment may be administered to a subject having a medical disorder or who ultimately may acquire the disorder, in order to prevent, cure, delay, reduce the severity of, and/or ameliorate one or more symptoms of a disorder or recurring disorder, or in order to prolong the survival of a subject beyond that expected in the absence of such treatment.

The term “effective amount” refers to a dosage or amount that is sufficient to regulate an activity to ameliorate clinical symptoms or achieve a desired biological outcome, e.g., decreased T cell and/or B cell activity, suppression of autoimmunity, suppression of transplant rejection, etc.

The term “in combination” in the context of treatment with two agents means that the agents are given substantially contemporaneously, either simultaneously or sequentially. If given sequentially, at the onset of administration of the second compound, the first of the two compounds is preferably still detectable at effective concentrations at the site of treatment.

The phrase “percent identical” or “percent identity” refers to the similarity between at least two different sequences. This percent identity can be determined by standard alignment algorithms, for example, the Basic Local Alignment Tool (BLAST) described by Altshul et al. ((1990) J. Mol. Biol., 215: 403-410); the algorithm of Needleman et al. ((1970) J. Mol. Biol., 48: 444-453); or the algorithm of Meyers et al. ((1988) Comput. Appl. Biosci., 4: 11-17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM 120 weight residue table, a gap length penalty of 12 and a gap penalty of 4. The percent identity is usually calculated by comparing sequences of similar length.

In certain embodiments, sequences similar or homologous (e.g., at least about 85% sequence identity) to the sequences disclosed are provided. In certain embodiments, the sequence identity can be about 90%, 91%, 92%, 93%, 94%, 95%, 96%, 97%, 98%, 99% or higher. Alternatively, substantial identity exists when the nucleic acid segments will hybridize under selective hybridization conditions (e.g., highly stringent hybridization conditions), to the complement of the strand. The nucleic acids may be present in whole cells, in a cell lysate, or in a partially purified or substantially pure form.

Isolated polynucleotides may be used as hybridization probes and primers to identify and isolate nucleic acids having sequences identical to or similar to those encoding the disclosed polynucleotides. Polynucleotides isolated in this fashion may be used, for example and not limitation, to produce antibodies against IL-22 or other IL-10-like cytokines or to identify cells expressing such antibodies. Hybridization methods for identifying and isolating nucleic acids include Southern hybridizations, in situ hybridization and Northern hybridization, and are well known to those skilled in the art.

Hybridization reactions can be performed under conditions of different stringencies. Preferably, each hybridizing polynucleotide hybridizes to its corresponding polynucleotide under reduced stringency conditions, more preferably stringent conditions, and most preferably highly stringent conditions. Examples of stringency conditions are shown in Table 1 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 1

| | | Hybrid | Hybridization | Wash |
|---|---|---|---|---|
| | | Length | Temperature and | Temperature |
| Condition | Hybrid | (bp)¹ | Buffer² | and Buffer² |
| A | DNA:DNA | >50 | 65° C.; 1X SSC -or- | 65° C.; 0.3X SSC |
| | | | 42° C.; 1X SSC, | |
| | | | 50% formamide | |
| B | DNA:DNA | <50 | T_{B}*; 1X SSC | T_{B}*; 1X SSC |
| C | DNA:RNA | >50 | 67° C.; 1X SSC -or- | 67° C.; 0.3X SSC |
| | | | 45° C.; 1X SSC, | |
| | | | 50% formamide | |
| D | DNA:RNA | <50 | T_{D}*; 1X SSC | T_{D}*; 1X SSC |
| E | RNA:RNA | >50 | 70° C.; 1X SSC -or- | 70° C.; 0.3X SSC |
| | | | 50° C.; 1X SSC, | |
| | | | 50% formamide | |
| F | RNA:RNA | <50 | T_{F}*; 1X SSC | T_{F}*; 1X SSC |
| G | DNA:DNA | >50 | 65° C.; 4X SSC -or- | 65° C.; 1X SSC |
| | | | 42° C.; 4X SSC, | |
| | | | 50% formamide | |
| H | DNA:DNA | <50 | T_{H}*; 4X SSC | T_{H}*; 4X SSC |
| I | DNA:RNA | >50 | 67° C.; 4X SSC -or- | 67° C.; 1X SSC |
| | | | 45° C.; 4X SSC, | |
| | | | 50% formamide | |
| J | DNA:RNA | <50 | T_{J}*; 4X SSC | T_{J}*; 4X SSC |
| K | RNA:RNA | >50 | 70° C.; 4X SSC -or- | 67° C.; 1X SSC |
| | | | 50° C.; 4X SSC, | |
| | | | 50% formamide | |
| L | RNA:RNA | <50 | T_{L}*; 2X SSC | T_{L}*; 2X SSC |
| M | DNA:DNA | >50 | 50° C.; 4X SSC -or- | 50° C.; 2X SSC |
| | | | 40° C.; 6X SSC, | |
| | | | 50% formamide | |
| N | DNA:DNA | <50 | T_{N}*; 6X SSC | T_{N}*; 6X SSC |
| O | DNA:RNA | >50 | 55° C.; 4X SSC -or- | 55° C.; 2X SSC |
| | | | 42° C.; 6X SSC, | |
| | | | 50% formamide | |
| P | DNA:RNA | <50 | T_{P}*; 6X SSC | T_{P}*; 6X SSC |
| Q | RNA:RNA | >50 | 60° C.; 4X SSC -or- | 60° C.; 2X SSC |
| | | | 45° C.; 6X SSC, | |
| | | | 50% formamide | |
| R | RNA:RNA | <50 | T_{R}*; 4X SSC | T_{R}*; 4X SSC |

¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.
²SSPE (1xSSPE is 0.15M NaC1, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 minutes after hybridization is complete.
T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, Tₘ(° C.) = 81.5 + 16.6(log₁₀Na⁺) + 0.41(% G + C) − (600/N), where N is the number of bases in the hybrid, and Na⁺ is the concentration of sodium ions in the hybridization buffer (Na⁺ for 1X SSC = 0.165M).

Additional examples of stringency conditions for polynucleotide hybridization are provided in Sambrook et al., Molecular Cloning: A Laboratory Manual, Chs. 9 & 11, Cold Spring Harbor Laboratory Press, Cold Spring Harbor, N.Y. (1989), and Ausubel et al., eds., Current Protocols in Molecular Biology, Sects. 2.10 & 6.3-6.4, John Wiley & Sons, Inc. (1995), herein incorporated by reference.

Isolated polynucleotides may be used as hybridization probes and primers to identify and isolate DNAs having sequences encoding allelic variants of the disclosed polynucleotides. Allelic variants are naturally occurring alternative forms of the disclosed polynucleotides that encode polypeptides that are identical to or have significant similarity to the polypeptides encoded by the disclosed polynucleotides. Preferably, allelic variants have at least 90%, 91%, 92%, 93%, 94%, 95%, 96%, 97%, 98%, or 99% sequence identity with the disclosed polynucleotides.

Isolated polynucleotides may also be used as hybridization probes and primers to identify and isolate DNAs having sequences encoding polypeptides homologous to the disclosed polynucleotides. These homologs are polynucleotides and polypeptides isolated from a different species than that of the disclosed polypeptides and polynucleotides, or within the same species, but with significant sequence similarity to the disclosed polynucleotides and polypeptides. In certain embodiments, polynucleotide homologs have at least 50%, 75%, 90%, 91%, 92%, 93%, 94%, 95%, 96%, 97%, 98%, or 99% identity with the disclosed polynucleotides, whereas polypeptide homologs have at least 30%, 45%, or 60% identity with the disclosed antibodies/polypeptides. In certain embodiments, homologs of the disclosed polynucleotides and polypeptides are those isolated from mammalian species.

Isolated polynucleotides may also be used as hybridization probes and primers to identify cells and tissues that express proteins, including antibodies, and the conditions under which they are expressed.

It is understood that the polypeptides and antagonists, e.g., antibodies, may have additional conservative or non-essential amino acid substitutions, which do not have a substantial effect on their functions. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

IL-22 nucleotide and amino acid sequences are described in U.S. Pat. No. 7,307,161 and provided below. The nucleotide sequence of each clone can also be determined by sequencing of the deposited clone in accordance with known methods. As used herein, a “secreted” protein is one which, when expressed in a suitable host cell, is transported across or through a membrane, including transport as a result of signal sequences in its amino acid sequence. “Secreted” proteins include without limitation proteins secreted wholly (e.g., soluble proteins) or partially (e.g., receptors) from the cell in which they are expressed. “Secreted” proteins also include without limitation proteins that are transported across the membrane of the endoplasmic reticulum.

Any form of IL-22 proteins less than full length can be used in the methods and compositions of the present claims. IL-22 fragments, e.g., IL-22 proteins of less than full length, can be produced by expressing a corresponding fragment of the polynucleotide encoding the full-length IL-22 protein in a host cell. Modified polynucleotides as described above may be made by standard molecular biology techniques, including construction of appropriate desired deletion mutants, site-directed mutagenesis methods, or by the polymerase chain reaction using appropriate oligonucleotide primers.

Any form of IL-1F6 proteins less than full length can be used in the methods and compositions of the present claims. IL-1F6 fragments, e.g., IL-1F6 proteins of less than full length, can be produced by expressing a corresponding fragment of the polynucleotide encoding the full-length IL-F6 protein in a host cell. Modified polynucleotides as described above may be made by standard molecular biology techniques, including construction of appropriate desired deletion mutants, site-directed mutagenesis methods, or by the polymerase chain reaction using appropriate oligonucleotide primers.

Any form of IL-1F8 proteins less than full length can be used in the methods and compositions of the present claims. IL-1F8 fragments, e.g., IL-1F8 proteins of less than full length, can be produced by expressing a corresponding fragment of the polynucleotide encoding the full-length IL-1F8 protein in a host cell. Modified polynucleotides as described above may be made by standard molecular biology techniques, including construction of appropriate desired deletion mutants, site-directed mutagenesis methods, or by the polymerase chain reaction using appropriate oligonucleotide primers.

Any form of IL-1F9 proteins less than full length can be used in the methods and compositions of the present claims. IL-1F9 fragments, e.g., IL-1F9 proteins of less than full length, can be produced by expressing a corresponding fragment of the polynucleotide encoding the full-length IL-1F9 protein in a host cell. Modified polynucleotides as described above may be made by standard molecular biology techniques, including construction of appropriate desired deletion mutants, site-directed mutagenesis methods, or by the polymerase chain reaction using appropriate oligonucleotide primers.

Any form of IL-1Rrp2 proteins less than full length can be used in the methods and compositions of the present claims. IL-1Rrp2 fragments, e.g., IL-1Rrp2 proteins of less than full length, can be produced by expressing a corresponding fragment of the polynucleotide encoding the full-length IL-1Rrp2 protein in a host cell. Modified polynucleotides as described above may be made by standard molecular biology techniques, including construction of appropriate desired deletion mutants, site-directed mutagenesis methods, or by the polymerase chain reaction using appropriate oligonucleotide primers.

Any form of IL-17A proteins less than full length can be used in the methods and compositions of the present claims. IL-17A fragments, e.g., IL-17A proteins of less than full length, can be produced by expressing a corresponding fragment of the polynucleotide encoding the full-length IL-17A protein in a host cell. Modified polynucleotides as described above may be made by standard molecular biology techniques, including construction of appropriate desired deletion mutants, site-directed mutagenesis methods, or by the polymerase chain reaction using appropriate oligonucleotide primers.

Fragments of the protein can be in linear form, or they can be cyclized using known methods, for example, as described in H. U. Saragovi, et al., Bio/Technology 10, 773-778 (1992) and in R. S. McDowell, et al., J. Amer. Chem. Soc. 114, 9245-9253 (1992), both of which are incorporated herein by reference. Such fragments can be fused to carrier molecules such as immunoglobulins for many purposes, including increasing the valency of protein binding sites. For example, fragments of the protein can be fused through “linker” sequences to the Fc portion of an immunoglobulin. For a bivalent form of the protein, the fusion can be to the Fc portion of an IgG molecule. Other immunoglobulin isotypes may be used to generate such fusions. For example, a protein-1 gM fusion can be used to generate a decavalent form of the protein.

IL-22 proteins and fragments thereof include proteins with amino acid sequence lengths that are at least 25% (more preferably at least 50%, and most preferably at least 75%) of the length of a disclosed protein and have at least 60% sequence identity (more preferably, at least 75% identity; most preferably at least 90%, 91%, 92%, 93%, 94%, 95%, 96%, 97%, 98%, 99%, or 100% identity along the length of the fragment) with that disclosed protein, where sequence identity is determined by comparing the amino acid sequences of the proteins when aligned so as to maximize overlap and identity while minimizing sequence gaps. In certain embodiments, proteins and protein fragments contain a segment comprising 8 or more (more preferably 20 or more, most preferably 30 or more) contiguous amino acids that shares at least 75% sequence identity (more preferably, at least 85% identity; most preferably at least 91%, 92%, 93%, 94%, 95%, 96%, 97%, 98%, or 99% identity) with any such segment of any of the disclosed proteins.

IL-1F6 proteins and fragments thereof include proteins with amino acid sequence lengths that are at least 25% (more preferably at least 50%, and most preferably at least 75%) of the length of a disclosed protein and have at least 60% sequence identity (more preferably, at least 75% identity; most preferably at least 90%, 91%, 92%, 93%, 94%, 95%, 96%, 97%, 98%, 99%, or 100% identity along the length of the fragment) with that disclosed protein, where sequence identity is determined by comparing the amino acid sequences of the proteins when aligned so as to maximize overlap and identity while minimizing sequence gaps. In certain embodiments, proteins and protein fragments contain a segment comprising 8 or more (more preferably 20 or more, most preferably 30 or more) contiguous amino acids that shares at least 75% sequence identity (more preferably, at least 85% identity; most preferably at least 91%, 92%, 93%, 94%, 95%, 96%, 97%, 98%, or 99% identity) with any such segment of any of the disclosed proteins.

IL-1F8 proteins and fragments thereof include proteins with amino acid sequence lengths that are at least 25% (more preferably at least 50%, and most preferably at least 75%) of the length of a disclosed protein and have at least 60% sequence identity (more preferably, at least 75% identity; most preferably at least 90%, 91%, 92%, 93%, 94%, 95%, 96%, 97%, 98%, 99%, or 100% identity along the length of the fragment) with that disclosed protein, where sequence identity is determined by comparing the amino acid sequences of the proteins when aligned so as to maximize overlap and identity while minimizing sequence gaps. In certain embodiments, proteins and protein fragments contain a segment comprising 8 or more (more preferably 20 or more, most preferably 30 or more) contiguous amino acids that shares at least 75% sequence identity (more preferably, at least 85% identity; most preferably at least 91%, 92%, 93%, 94%, 95%, 96%, 97%, 98%, or 99% identity) with any such segment of any of the disclosed proteins.

IL-1F9 proteins and fragments thereof include proteins with amino acid sequence lengths that are at least 25% (more preferably at least 50%, and most preferably at least 75%) of the length of a disclosed protein and have at least 60% sequence identity (more preferably, at least 75% identity; most preferably at least 90%, 91%, 92%, 93%, 94%, 95%, 96%, 97%, 98%, 99%, or 100% identity along the length of the fragment) with that disclosed protein, where sequence identity is determined by comparing the amino acid sequences of the proteins when aligned so as to maximize overlap and identity while minimizing sequence gaps. In certain embodiments, proteins and protein fragments contain a segment comprising 8 or more (more preferably 20 or more, most preferably 30 or more) contiguous amino acids that shares at least 75% sequence identity (more preferably, at least 85% identity; most preferably at least 91%, 92%, 93%, 94%, 95%, 96%, 97%, 98%, or 99% identity) with any such segment of any of the disclosed proteins.

IL-1Rrp2 proteins and fragments thereof include proteins with amino acid sequence lengths that are at least 25% (more preferably at least 50%, and most preferably at least 75%) of the length of a disclosed protein and have at least 60% sequence identity (more preferably, at least 75% identity; most preferably at least 90%, 91%, 92%, 93%, 94%, 95%, 96%, 97%, 98%, 99%, or 100% identity along the length of the fragment) with that disclosed protein, where sequence identity is determined by comparing the amino acid sequences of the proteins when aligned so as to maximize overlap and identity while minimizing sequence gaps. In certain embodiments, proteins and protein fragments contain a segment comprising 8 or more (more preferably 20 or more, most preferably 30 or more) contiguous amino acids that shares at least 75% sequence identity (more preferably, at least 85% identity; most preferably at least 91%, 92%, 93%, 94%, 95%, 96%, 97%, 98%, or 99% identity) with any such segment of any of the disclosed proteins.

IL-17A proteins and fragments thereof include proteins with amino acid sequence lengths that are at least 25% (more preferably at least 50%, and most preferably at least 75%) of the length of a disclosed protein and have at least 60% sequence identity (more preferably, at least 75% identity; most preferably at least 90%, 91%, 92%, 93%, 94%, 95%, 96%, 97%, 98%, 99%, or 100% identity along the length of the fragment) with that disclosed protein, where sequence identity is determined by comparing the amino acid sequences of the proteins when aligned so as to maximize overlap and identity while minimizing sequence gaps. In certain embodiments, proteins and protein fragments contain a segment comprising 8 or more (more preferably 20 or more, most preferably 30 or more) contiguous amino acids that shares at least 75% sequence identity (more preferably, at least 85% identity; most preferably at least 91%, 92%, 93%, 94%, 95%, 96%, 97%, 98%, or 99% identity) with any such segment of any of the disclosed proteins.

Recombinant polynucleotides can be operably linked to an expression control sequence such as, for example and not limitation, the pMT2 or pED expression vectors disclosed in Kaufman et al., Nucleic Acids Res. 19, 4485-4490 (1991), to produce the protein recombinantly. Many suitable expression control sequences are known in the art. General methods of expressing recombinant proteins are also known and are exemplified in R. Kaufman (1990) Methods in Enzymology 185, 537-566. As defined herein “operably linked” means that the isolated polynucleotide and an expression control sequence are situated within a vector or cell in such a way that the protein is expressed by a host cell which has been transformed (transfected) with the ligated polynucleotide/expression control sequence.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids.

The term “regulatory sequence” as used herein includes promoters, enhancers and other expression control elements (e.g., polyadenylation signals) that control the transcription or translation of the antibody chain genes. Such regulatory sequences are described, for example, in Goeddel; Gene Expression Technology: Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, etc. Regulatory sequences for mammalian host cell expression include, but are not limited to, viral elements that direct high levels of protein expression in mammalian cells, such as promoters and/or enhancers derived from FF-la promoter and BGH poly A, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al.

In certain embodiments, recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr host cells with methotrexate selection/amplification) and the neo gene (for G418 selection).

A number of types of cells may act as suitable host cells for expression of a protein (or fusion protein). Any cell type capable of expressing functional IL-22 protein may be used. Suitable mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK, Rat2, BaF3, 32D, FDCP-1, PC12, M1x or C2C12 cells.

In certain embodiments, a protein or fusion protein may also be produced by operably linking an isolated polynucleotide to suitable control sequences in one or more insect expression vectors, and employing an insect expression system. Materials and methods for baculovirus/insect cell expression systems are commercially available in kit form from, e.g., Invitrogen, San Diego, Calif. U.S.A. (the MaxBac® kit), and such methods are well known in the art, as described in Summers and Smith, Texas Agricultural Experiment Station Bulletin No. 1555 (1987), incorporated herein by reference. Soluble forms of the IL-22 protein may also be produced in insect cells using appropriate isolated polynucleotides as described above.

Alternatively, the protein or fusion protein may be produced in lower eukaryotes such as yeast or in prokaryotes such as bacteria. Suitable yeast strains include Saccharomyces cerevisiae, Schizosaccharomyces pombe, Kluyveromyces strains, Candida, or any yeast strain capable of expressing heterologous proteins. Suitable bacterial strains include Escherichia coli, Bacillus subtilis, Salmonella typhimurium, or any bacterial strain capable of expressing heterologous proteins.

Expression in bacteria may result in formation of inclusion bodies incorporating the recombinant protein. Thus, refolding of the recombinant protein may be required in order to produce active or more active material. Several methods for obtaining correctly folded heterologous proteins from bacterial inclusion bodies are known in the art. These methods generally involve solubilizing the protein from the inclusion bodies, then denaturing the protein completely using a chaotropic agent. When cysteine residues are present in the primary amino acid sequence of the protein, it is often necessary to accomplish the refolding in an environment which allows correct formation of disulfide bonds (a redox system). General methods of refolding are disclosed in Kohno, Meth. Enzym., 185:187-195 (1990). EP 0433225 and copending application U.S. Ser. No. 08/163,877 describe other appropriate methods.

A protein or fusion protein may also be expressed as a product of transgenic animals, e.g., as a component of the milk of transgenic cows, goats, pigs, or sheep which are characterized by somatic or germ cells containing a polynucleotide sequence encoding the protein or fusion protein.

The protein or fusion protein may be prepared by growing a culture transformed host cells under culture conditions necessary to express the desired protein. The resulting expressed protein may then be purified from the culture medium or cell extracts. Soluble forms of the protein or fusion protein can be purified from conditioned media. In certain embodiments, membrane-bound forms of protein can be purified by preparing a total membrane fraction from the expressing cell and extracting the membranes with a non-ionic detergent such as Triton X-100.

In certain embodiments, the protein can be purified using methods known to those skilled in the art. For example, and not limitation, the protein can be concentrated using a commercially available protein concentration filter, including, but not limited to, an Amicon or Millipore Pellicon ultrafiltration unit. Following the concentration step, the concentrate can be applied to a purification matrix such as a gel filtration medium. Alternatively, an anion exchange resin can be employed, for example, a matrix or substrate having pendant diethylaminoethyl (DEAB) or polyetheyleneimine (PEI) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. In certain embodiments, sulfopropyl groups are preferred (e.g., S-Sepharose® columns). The purification of the protein or fusion protein from culture supernatant may also include one or more column steps over such affinity resins as concanavalin A-agarose, heparin-toyopearl® or Cibacrom blue 3GA Sepharose®; or by hydrophobic interaction chromatography using such resins as phenyl ether, butyl ether, or propyl ether; or by immunoaffinity chromatography. Finally, one or more reverse-phase high performance liquid chromatography (RP-HPLC) steps employing hydrophobic RP-HPLC media, e.g., silica gel having pendant methyl or other aliphatic groups, can be employed to further purify the protein. Affinity columns including antibodies to the protein can also be used in purification in accordance with known methods. Some or all of the foregoing purification steps, in various combinations or with other known methods, can also be employed to provide a substantially purified isolated recombinant protein. Preferably, the isolated protein is purified so that it is substantially free of other mammalian proteins.

Polypeptides may also be produced by known conventional chemical synthesis. Methods for constructing proteins by synthetic means are known to those skilled in the art. The synthetically-constructed protein sequences, by virtue of sharing primary, secondary or tertiary structural and/or conformational characteristics with proteins may possess biological properties in common therewith, including protein activity. Thus, they can be employed as biologically active or immunological substitutes for natural, purified proteins in screening of therapeutic compounds and in immunological processes for the development of antibodies.

Antibodies, also known as immunoglobulins, are typically tetrameric glycosylated proteins composed of two light (L) chains of approximately 25 kDa each and two heavy (H) chains of approximately 50 kDa each. Two types of light chain, termed lambda and kappa, may be found in antibodies. Depending on the amino acid sequence of the constant domain of heavy chains, immunoglobulins can be assigned to five major classes: A, D, E, G, and M, and several of these may be further divided into subclasses (isotypes), e.g., IgG₁, IgG₂, IgG₃, IgG₄, IgA₁, and IgA₂. Each light chain includes an N-terminal variable (V) domain (VL) and a constant (C) domain (CL). Each heavy chain includes an N-terminal V domain (VH), three or four C domains (CHs), and a hinge region. The CH domain most proximal to VH is designated as CH1. The VH and VL domains consist of four regions of relatively conserved sequences called framework regions (FR1, FR2, FR3, and FR4), which form a scaffold for three regions of hypervariable sequences (complementarity determining regions, CDRs). The CDRs contain most of the residues responsible for specific interactions of the antibody with the antigen. CDRs are referred to as CDR1, CDR2, and CDR3. Accordingly, CDR constituents on the heavy chain are referred to as H1, H2, and H3, while CDR constituents on the light chain are referred to as L1, L2, and L3.

CDR3 is typically the greatest source of molecular diversity within the antibody-binding site. H3, for example, can be as short as two amino acid residues or greater than 26 amino acids. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988. One of skill in the art will recognize that each subunit structure, e.g., a CH, VH, CL, VL, CDR, FR structure, comprises active fragments, e.g., the portion of the VH, VL, or CDR subunit the binds to the antigen, i.e., the antigen-binding fragment, or, e.g., the portion of the CH subunit that binds to and/or activates, e.g., an Fc receptor and/or complement. The CDRs typically refer to the Kabat CDRs, as described in Sequences of Proteins of Immunological Interest, US Department of Health and Human Services (1991), eds. Kabat et al. Another standard for characterizing the antigen binding site is to refer to the hypervariable loops as described by Chothia. See, e.g., Chothia, D. et al. (1992) J. Mol. Biol. 227:799-817; and Tomlinson et al. (1995) EMBO J. 14:4628-4638. Still another standard is the AbM definition used by Oxford Molecular's AbM antibody modelling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). Embodiments described with respect to Kabat CDRs can alternatively be implemented using similar described relationships with respect to Chothia hypervariable loops or to the AbM-defined loops.

The Fab fragment (Fragment antigen-binding) consists of V_{H}-C_{H}1 and V_{L}-C_{L} domains covalently linked by a disulfide bond between the constant regions. The Fᵥ fragment is smaller and consists of V_{H} and V_{L} domains non-covalently linked. To overcome the tendency of non-covalently linked domains to dissociate, a single chain Fᵥ fragment (scFᵥ) can be constructed. The scFᵥ contains a flexible polypeptide that links (1) the C-terminus of V_{H} to the N-terminus of V_{L}, or (2) the C-terminus of V_{L} to the N-terminus of V_{H}. A 15-mer (Gly₄Ser)₃ peptide may be used as a linker, but other linkers are known in the art.

The sequence of antibody genes after assembly and somatic mutation is highly varied, and these varied genes are estimated to encode 10¹⁰ different antibody molecules (Immunoglobulin Genes, 2nd ed., eds. Jonio et al., Academic Press, San Diego, Calif., 1995).

Numerous methods known to those skilled in the art are available for obtaining antibodies or antigen-binding fragments thereof. For example, antibodies can be produced using recombinant DNA methods (U.S. Pat. No. 4,816,567). Monoclonal antibodies may also be produced by generation of hybridomas (see e.g., Kohler and Milstein (1975) Nature, 256: 495-499) in accordance with known methods. Hybridomas formed in this manner are then screened using standard methods, such as enzyme-linked immunosorbent assay (ELISA) and surface plasmon resonance (BIACORE™) analysis, to identify one or more hybridomas that produce an antibody that specifically binds with a specified antigen. Any form of the specified antigen may be used as the immunogen, e.g., recombinant antigen, naturally occurring forms, any variants or fragments thereof, as well as antigenic peptide thereof.

One exemplary method of making antibodies includes screening protein expression libraries, e.g., phage or ribosome display libraries. Phage display is described, for example, in Ladner et al., U.S. Pat. No. 5,223,409; Smith (1985) Science 228:1315-1317; Clackson et al. (1991) Nature, 352: 624-628; Marks et al. (1991) J. Mol. Biol., 222: 581-597WO92/18619; WO 91/17271; WO 92/20791; WO 92/15679; WO 93/01288; WO 92/01047; WO 92/09690; and WO 90/02809.

In addition to the use of display libraries, the specified antigen can be used to immunize a non-human animal, e.g., including, but not limited to, mouse, hamster, rat, monkey, camel, llama, fish, shark, goat, rabbit, and bovine. In certain embodiments, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XENOMOUSE™, Green et al. (1994) Nature Genetics 7:13-21, US 2003-0070185, WO 96/34096, published Oct. 31, 1996, and PCT Application No. PCT/US96/05928, filed Apr. 29, 1996.

In certain embodiments, a monoclonal antibody is obtained from a non-human animal, e.g., including, but not limited to, mouse, hamster, rat, monkey, camel, llama, fish, shark, goat, rabbit, and bovine and then modified, e.g., humanized or deimmunized. In certain embodiments chimeric antibodies may be produced using recombinant DNA techniques known in the art. A variety of approaches for making chimeric antibodies have been described. See e.g., Morrison et al., Proc. Natl. Acad. Sci. U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., European Patent Publication EP171496; European Patent Publication 0173494, United Kingdom Patent GB 2177096B. Humanized antibodies may also be produced, for example, using transgenic mice that express human heavy and light chain genes, but are incapable of expressing the endogenous mouse immunoglobulin heavy and light chain genes. Winter describes an exemplary CDR-grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by Morrison (1985) Science 229:1202-1207; by Oi et al. (1986) BioTechniques 4:214; and by U.S. Pat. No. 5,585,089; U.S. Pat. No. 5,693,761; U.S. Pat. No. 5,693,762; U.S. Pat. No. 5,859,205; and U.S. Pat. No. 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

In certain embodiments, a humanized antibody is optimized by the introduction of conservative substitutions, consensus sequence substitutions, germline substitutions and/or backmutations. Such altered immunoglobulin molecules can be made by any of several techniques known in the art, (e.g., Teng et al., Proc. Natl. Acad. Sci. U.S.A., 80: 7308-7312, 1983; Kozbor et al., Immunology Today, 4: 7279, 1983; Olsson et al., Meth. Enzymol., 92: 3-16, 1982), and may be made according to the teachings of PCT Publication W092/06193 or EP 0239400).

An antibody or fragment thereof may also be modified by specific deletion of human T cell epitopes or “deimmunization” by the methods disclosed in WO 98/52976 and WO 00/34317. Briefly, the heavy and light chain variable domains of an antibody can be analyzed for peptides that bind to MHC Class II; these peptides represent potential T-cell epitopes (as defined in WO 98/52976 and WO 00/34317). For detection of potential T-cell epitopes, a computer modeling approach termed “peptide threading” can be applied, and in addition a database of human MHC class II binding peptides can be searched for motifs present in the V_{H} and V_{L} sequences, as described in WO 98/52976 and WO 00/34317. These motifs bind to any of the 18 major MHC class II DR allotypes, and thus constitute potential T cell epitopes. Potential T-cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. Human germline sequences, e.g., are disclosed in Tomlinson, et al. (1992) J. Mol. Biol. 227:776-798; Cook, G. P. et al. (1995) Immunol. Today Vol. 16 (5): 237-242; Chothia, D. et al. (1992) J. Mol. Biol. 227:799-817; and Tomlinson et al. (1995) EMBO J. 14:4628-4638. The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

In certain embodiments, an antibody can contain an altered immunoglobulin constant or Fc region. For example, an antibody produced in accordance with the teachings herein may bind more strongly or with more specificity to effector molecules such as complement and/or Fc receptors, which can control several immune functions of the antibody such as effector cell activity, lysis, complement-mediated activity, antibody clearance, and antibody half-life. Typical Fc receptors that bind to an Fc region of an antibody (e.g., an IgG antibody) include, but are not limited to, receptors of the FcyRI, FcyRII, and FcyRIII and FcRn subclasses, including allelic variants and alternatively spliced forms of these receptors. Fc receptors are reviewed in Ravetch and Kinet, Annu. Rev. Immunol 9:457-92, 1991; Capel et al., Immunomethods 4:25-34,1994; and de Haas et al., J. Lab. Clin. Med. 126:330-41, 1995).

For additional antibody production techniques, see Antibodies: A Laboratory Manual, eds. Harlow et al., Cold Spring Harbor Laboratory, 1988.

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992). In certain embodiments, the bispecific antibody comprises a first binding domain polypeptide, such as a Fab' fragment, linked via an immunoglobulin constant region to a second binding domain polypeptide.

Antibodies of the present invention can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. In one aspect of the invention, a single domain antibody can be derived from a variable region of the immunoglobulin found in fish, such as, for example, that which is derived from the immunoglobulin isotype known as Novel Antigen Receptor (NAR) found in the serum of shark. Methods of producing single domain antibodies derived from a variable region of NAR (“IgNARs”) are described in WO 03/014161 and Streltsov (2005) Protein Sci. 14:2901-2909.

According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The invention also contemplates the use of binding domain-immunoglobulin fusion proteins including a binding domain polypeptide that is fused or otherwise connected to an immunoglobulin hinge or hinge-acting region polypeptide, which in turn is fused or otherwise connected to a region comprising one or more native or engineered constant regions from an immunoglobulin heavy chain, other than CH1, for example, the CH2 and CH3 regions of IgG and IgA, or the CH3 and CH4 regions of IgE (see e.g., U.S. 2005/0136049 by Ledbetter, J. et al. for a more complete description). The binding domain-immunoglobulin fusion protein can further include a region that includes a native or engineered immunoglobulin heavy chain CH2 constant region polypeptide (or CH3 in the case of a construct derived in whole or in part from IgE) that is fused or otherwise connected to the hinge region polypeptide and a native or engineered immunoglobulin heavy chain CH3 constant region polypeptide (or CH4 in the case of a construct derived in whole or in part from IgE) that is fused or otherwise connected to the CH2 constant region polypeptide (or CH3 in the case of a construct derived in whole or in part from IgE). Typically, such binding domain-immunoglobulin fusion proteins are capable of at least one immunological activity selected from the group consisting of antibody dependent cell-mediated cytotoxicity, complement fixation, and/or binding to a target, for example, a target antigen.

In certain embodiments, therapeutic proteins, i.e., a protein or peptide that has a biological effect on a region in the body on which it acts or on a region of the body on which it remotely acts via intermediates, and method of designing and making these therapeutic proteins, are provided. Therapeutic proteins of the current invention can include peptide mimetics. Mimetics are peptide-containing molecules that mimic elements of protein secondary structure. See, for example, Johnson et al., “Peptide Turn Mimetics” in BIOTECHNOLOGY AND PHARMACY, Pezzuto et al., Eds., Chapman and Hall, New York (1993), incorporated herein by reference. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. In conjunction with the information provided by the current invention, these principles may be used to engineer second generation molecules having many of the natural properties of the targeting peptides disclosed herein. These second generation molecules can also be altered and provide potentially improved characteristics. Using the current invention, both protein and small molecule therapeutics can be designed to interrupt the desired cytokine activity by, for example, being specifically designed to bind at the desired positions, i.e., at the amino acid positions demonstrated as being important for a binding complex, and therefore effectively reducing or inhibiting the activity associated with the cytokine and its receptor or receptor complex.

Other embodiments of therapeutic proteins include fusion proteins. These molecules generally have all or a substantial portion of a targeting peptide, for example, IL-22 or an anti-IL-22 antibody, linked at the N- or C-terminus, to all or a portion of a second polypeptide or protein. For example, fusions may employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions. Examples of proteins or peptides that may be incorporated into a fusion protein include cytostatic proteins, cytocidal proteins, pro-apoptosis agents, anti-angiogenic agents, hormones, cytokines, growth factors, peptide drugs, antibodies, Fab fragments of antibodies, antigens, receptor proteins, enzymes, lectins, MHC proteins, cell adhesion proteins and binding proteins. Methods of generating fusion proteins are well known to those of skill in the art. Such proteins can be produced, for example, by chemical attachment using bifunctional cross-linking reagents, by de novo synthesis of the complete fusion protein, or by attachment of a DNA sequence encoding the targeting peptide to a DNA sequence encoding the second peptide or protein, followed by expression of the intact fusion protein.

In certain embodiments, the targeting peptide, for example, IL-22 or an anti-IL-22 antibody, is fused with an immunoglobulin heavy chain constant region, such as an Fc fragment, which contains two constant region domains and a hinge region but lacks the variable region (See, U.S. Pat. Nos. 6,018,026 and 5,750,375, incorporated herein by reference). The Fc region may be a naturally occurring Fc region, or may be altered to improve certain qualities, such as therapeutic qualities, circulation time, reduced aggregation, etc. Peptides and proteins fused to an Fc region typically exhibit a greater half-life in vivo than the unfused counterpart. Also, a fusion to an Fc region permits dimerization/multimerization of the fusion polypeptide.

In certain embodiments, mutagenesis is used to make an antibody more similar to one or more germline sequences. This may be desirable when mutations are introduced into the framework region of an antibody through somatic mutagenesis or through error prone PCR. Germline sequences for the V_{H} and V_{L} domains can be identified by performing amino acid and nucleic acid sequence alignments against the VBASE database (MRC Center for Protein Engineering, UK). VBASE is a comprehensive directory of all human germline variable region sequences compiled from over a thousand published sequences, including those in the current releases of the Genbank and EMBL data libraries. In some embodiments, the FR regions of the scFvs are mutated in conformity with the closest matches in the VBASE database and the CDR portions are kept intact.

Using recombinant DNA methodology, a disclosed CDR sequence may be introduced into a repertoire of V_{H} or V_{L} domains lacking the respective CDR (Marks et al. (BioTechnology (1992) 10: 779-783). For example, a primer adjacent to the 5′ end of the variable domain and a primer to the third FR can be used to generate a repertoire of variable domain sequences lacking CDR3. This repertoire can be combined with a CDR3 of a disclosed antibody. Using analogous techniques, portions of a disclosed CDR sequence may be shuffled with portions of CDR sequences from other antibodies to provide a repertoire of antigen-binding fragments that bind IL-22. Either repertoire can be expressed in a host system such as phage display (described in WO 92/01047 and its corresponding U.S. Pat. No. 5,969,108) so suitable antigen-binding fragments that bind to IL-22 can be selected.

A further alternative uses random mutagenesis of the disclosed V_{H} or V_{L} sequences to generate variant V_{H} or V_{L} domains still capable of binding IL-22. A technique using error-prone PCR is described by Gram et al. (Proc. Nat. Acad. Sci. U.S.A. (1992) 89: 3576-3580).

Another method uses direct mutagenesis of the disclosed V_{H} or V_{L} sequences. Such techniques are disclosed by Barbas et al. (Proc. Nat. Acad. Sci. U.S.A. (1994) 91: 3809-3813) and Schier et al. (J. Mol. Biol. (1996) 263: 551-567).

A portion of a variable domain will comprise at least one CDR region substantially as set out herein and, optionally, intervening framework regions from the V_{H} or V_{L} domains as set out herein. The portion may include the C-terminal half of FR1 and/or the N-terminal half of FR4. Additional residues at the N-terminal or C-terminal end of the variable domain may not be the same residues found in naturally occurring antibodies. For example, construction of antibodies by recombinant DNA techniques often introduces N- or C-terminal residues from its use of linkers. Some linkers may be used to join variable domains to other variable domains (e.g., diabodies), constant domains, or proteinaceous labels.

Antibodies can be modified to alter their glycosylation; that is, at least one carbohydrate moiety can be deleted or added to the antibody. Deletion or addition of glycosylation sites can be accomplished by changing amino acid sequence to delete or create glycosylation consensus sites, which are well known in the art. Another means of adding carbohydrate moieties is the chemical or enzymatic coupling of glycosides to amino acid residues of the antibody (see WO 87/05330 and Aplin et al. (1981) CRC Crit. Rev. Biochem., 22: 259-306). Removal of carbohydrate moieties can also be accomplished chemically or enzymatically (see Hakimuddin et al. (1987) Arch. Biochem. Biophys., 259: 52; Edge et al. (1981) Anal. Biochem., 118: 131; Thotakura et al. (1987) Meth. Enzymol., 138: 350).

Methods for altering an antibody constant region are known in the art. Antibodies with altered function (e.g., altered affinity for an effector ligand such as FcR on a cell or the Cl component of complement) can be produced by replacing at least one amino acid residue in the constant portion of the antibody with a different residue (see e.g., EP 388,151 A1, U.S. Pat. No. 5,624,821 and U.S. Pat. No. 5,648,260). Similar types of alterations could be described which if applied to a murine or other species antibody would reduce or eliminate similar functions.

For example, it is possible to alter the affinity of an Fc region of an antibody (e.g., an IgG, such as a human IgG) for FcR (e.g., Fc gamma R1) or Clq. The affinity may be altered by replacing at least one specified residue with at least one residue having an appropriate functionality on its side chain, or by introducing a charged functional group, such as glutamate or aspartate, or perhaps an aromatic non-polar residue such as phenylalanine, tyrosine, tryptophan or alanine (see e.g., U.S. Pat. No. 5,624,821).

In another example, replacing residue 297 (asparagine) with alanine in the IgG constant region significantly inhibits recruitment of effector cells, while only slightly reducing (about three fold weaker) affinity for Clq (see e.g., U.S. Pat. No. 5,624,821). The numbering of the residues in the heavy chain is that of the EU index (see Kabat et al., 1991 supra). This alteration destroys the glycosylation site and it is believed that the presence of carbohydrate is required for Fc receptor binding. Any other substitution at this site that destroys the glycosylation site is believed to cause a similar decrease in lytic activity. Other amino acid substitutions, e.g., changing any one of residues 318 (Glu), 320 (Lys) and 322 (Lys), to Ala, are also known to abolish Clq binding to the Fc region of IgG antibodies (see e.g., U.S. Pat. No. 5,624,821).

Modified antibodies can be produced which have a reduced interaction with an Fc receptor. For example, it has been shown that in human IgG₃, which binds to the human Fc gamma R1 receptor, changing Leu 235 to Glu destroys its interaction with the receptor. Mutations on adjacent or close sites in the hinge link region of an antibody (e.g., replacing residues 234, 236 or 237 with Ala) can also be used to affect antibody affinity for the Fc gamma R1 receptor. The numbering of the residues in the heavy chain is based in the EU index (see Kabat et al., 1991 supra).

Additional methods for altering the lytic activity of an antibody, for example, by altering at least one amino acid in the N-terminal region of the CH2 domain, are described in WO 94/29351 by Morgan et al. and U.S. Pat. No. 5,624,821.

In certain embodiments, antibodies may be tagged with a detectable or functional label. These labels include radiolabels (e.g., ¹³¹I or ⁹⁹Tc), enzymatic labels (e.g., horseradish peroxidase or alkaline phosphatase), and other chemical moieties (e.g., biotin).

In certain embodiments, the IL-22 antagonists are antibodies, or fragments thereof (e.g., antigen-binding fragments thereof), that bind to mammalian (e.g., human or murine) IL-22. In certain embodiments, the anti-IL-22 antibody or fragment thereof (e.g., an Fab, F(ab′)2, Fv or a single chain Fv fragment) is a monoclonal or single specificity antibody. The antibody or fragment thereof can also be a human, humanized, chimeric, or in vitro generated antibody against human IL-22.

In certain embodiments, the IL-1F6 antagonists are antibodies, or fragments thereof (e.g., antigen-binding fragments thereof), that bind to mammalian (e.g., human or murine) IL-1F6. In certain embodiments, the anti-IL-1F6 antibody or fragment thereof (e.g., an Fab, F(ab′)2, Fv or a single chain Fv fragment) is a monoclonal or single specificity antibody. The antibody or fragment thereof can also be a human, humanized, chimeric, or in vitro generated antibody against human IL-1F6.

In certain embodiments, the IL-1F8 antagonists are antibodies, or fragments thereof (e.g., antigen-binding fragments thereof), that bind to mammalian (e.g., human or murine) IL-1F8. In certain embodiments, the anti-IL-1F8 antibody or fragment thereof (e.g., an Fab, F(ab′)2, Fv or a single chain Fv fragment) is a monoclonal or single specificity antibody. The antibody or fragment thereof can also be a human, humanized, chimeric, or in vitro generated antibody against human IL-1F8.

In certain embodiments, the IL-1F9 antagonists are antibodies, or fragments thereof (e.g., antigen-binding fragments thereof), that bind to mammalian (e.g., human or murine) IL-1F9. In certain embodiments, the anti-IL-1F9 antibody or fragment thereof (e.g., an Fab, F(ab')2, Fv or a single chain Fv fragment) is a monoclonal or single specificity antibody. The antibody or fragment thereof can also be a human, humanized, chimeric, or in vitro generated antibody against human IL-1F9.

In certain embodiments, the IL-1Rrp2 antagonists are antibodies, or fragments thereof (e.g., antigen-binding fragments thereof), that bind to mammalian (e.g., human or murine) IL-1Rrp2. In certain embodiments, the anti-IL-1Rrp2 antibody or fragment thereof (e.g., an Fab, F(ab′)2, Fv or a single chain Fv fragment) is a monoclonal or single specificity antibody. The antibody or fragment thereof can also be a human, humanized, chimeric, or in vitro generated antibody against human IL-1Rrp2.

In certain embodiments, the IL-17A antagonists are antibodies, or fragments thereof (e.g., antigen-binding fragments thereof), that bind to mammalian (e.g., human or murine) IL-17A. In certain embodiments, the anti-IL-17A antibody or fragment thereof (e.g., an Fab, F(ab′)2, Fv or a single chain Fv fragment) is a monoclonal or single specificity antibody. The antibody or fragment thereof can also be a human, humanized, chimeric, or in vitro generated antibody against human IL-17A.

In certain embodiments, the TNFα antagonists are antibodies, or fragments thereof (e.g., antigen-binding fragments thereof), that bind to mammalian (e.g., human or murine) TNFα. In certain embodiments, the anti-TNFα antibody or fragment thereof (e.g., an Fab, F(ab′)2, Fv or a single chain Fv fragment) is a monoclonal or single specificity antibody. The antibody or fragment thereof can also be a human, humanized, chimeric, or in vitro generated antibody against human TNFα.

Examples of TNFα antagonists include antibodies to TNF (e.g., human TNFα), such as D2E7 (human anti- TNFα antibody, U.S. Pat. No. 6,258,562, Humira™, BASF); CDP-571/CDP-870/BAY-10-3356 (humanized anti- TNFα antibodies, Celltech/Pharmacia); cA2 (chimeric anti- TNFα antibody, Remicade™, Centocor); and anti-TNF antibody fragments (e.g., CPD870). Other examples include soluble TNF receptor (e.g., human p55 or p75) fragments and derivatives, such as p55 kdTNFR-IgG (55 kD TNF receptor-IgG fusion protein, Lenercept™) and 75 kdTNFR-IgG (75 kD TNF receptor-IgG fusion protein, Enbrel^{TM}, Immunex, see, e.g., Arthritis & Rheumatism (1994) Vol. 37, S295; J. Invest. Med. (1996) Vol. 44, 235A). Further examples include enzyme antagonists (e.g., TNFα converting enzyme inhibitors (TACE) such as alpha-sulfonyl hydroxamic acid derivative (WO 01/55112) or N-hydroxyformamide inhibitor (GW 3333, -005, or -022)) and TNF-bp/s-TNFR (soluble TNF binding protein, see e.g., Arthritis & Rheumatism (1996) Vol. 39, No. 9 (supplement), S284; and Am. J. Physiol. Heart Circ. Physiol. (1995) Vol. 268, pp. 37-42). TNF antagonists may be soluble TNF receptor (e.g., human p55 or p75) fragments and derivatives, such as 75 kdTNFR-IgG; and TNFα converting enzyme (TACE) inhibitors.

The production of anti-IL-22 antibodies is described in more detail in U.S. Published Patent Application Nos. 2005-0042220 and 2007-0243589. One non-limiting example of an anti-IL22 antibody that interferes with IL-22 binding to IL-22R is referred to as “Ab-04” or “IL22-04” in US Published Patent Application No. 2005-0042220. Ab-04 (also referred to herein as rat monoclonal antibody “P3/2”) binds to human IL-22 and neutralizes human IL-22 activity. A hybridoma cell line producing Ab-04 has been deposited with the ATCC on Jun. 5, 2003 and has been assigned ATCC accession number PTA-5255. Another non-limiting example of an anti-IL22 antibody that interferes with IL-22 binding to IL-10R2 is “Ab-02” or “IL22-02.” Ab-02 (also referred to herein as rat monoclonal antibody “P3/3”) binds to mouse and human IL-22 and neutralizes the activity of mouse and human IL-22. A hybridoma cell line producing Ab-02 has been deposited on Jun. 5, 2003 with the ATCC and has been assigned ATCC accession number PTA-5254. Additional examples of IL-22 antibodies that reduce, inhibit or antagonize IL-22 activity are found in U.S. Published Patent Application No. 2007-0243589, which describes germlined antibodies identified as GIL01, GIL16, GIL45, GIL60, GIL68, GIL92, 062A09, 087B03, 166B06, 166G05, 354A08, 355B06, 355E04, 356A11, and 368D04.

Antibodies may also be used to detect the presence of one or more molecules, such as, but not limited to, IL-22, IL-1F6, IL-1F8, IL-1F9, and IL-17A, in biological samples. By correlating the presence or level of these proteins with a medical condition, one of skill in the art can diagnose the associated medical condition. For example, IL-22 induces changes associated with those caused by inflammatory cytokines (such as IL-1 and TNFα), and inhibitors of IL-22 ameliorate symptoms of rheumatoid arthritis (WO 2005/000897 A2). Exemplary medical conditions that may be diagnosed by the antibodies include, but are not limited to, multiple sclerosis, rheumatoid arthritis, psoriasis, lupus, inflammatory bowel disease, pancreatitis, and transplant rejection.

Certain methods described in this application utilize compositions suitable for pharmaceutical use and administration to patients. These compositions comprise a pharmaceutical excipient and one or more antibodies, one or more soluble receptors, one or more binding proteins, or combinations of those antibodies, soluble receptors, and/or binding proteins. As used herein, “pharmaceutical excipient” includes solvents, dispersion media, coatings, antibacterial and antifungal agents, isotonic and absorption delaying agents, etc., that are compatible with pharmaceutical administration. Use of these agents for pharmaceutically active substances is well known in the art. The compositions may also contain other active compounds providing supplemental, additional, or enhanced therapeutic functions. The pharmaceutical compositions may also be included in a container, pack, or dispenser together with instructions for administration.

A pharmaceutical composition can be formulated to be compatible with its intended route of administration. Methods to accomplish the administration are known to those of ordinary skill in the art. It may also be possible to create compositions which may be topically or orally administered, or which may be capable of transmission across mucous membranes. For example, the administration may be intravenous, intraperitoneal, intramuscular, intracavity, subcutaneous, cutaneous, or transdermal.

Solutions or suspensions used for intradermal or subcutaneous application typically include at least one of the following components: a sterile diluent such as water, saline solution, fixed oils, polyethylene glycol, glycerine, propylene glycol, or other synthetic solvent; antibacterial agents such as benzyl alcohol or methyl parabens; antioxidants such as ascorbic acid or sodium bisulfate; chelating agents such as ethylenediaminetetraacetic acid (EDTA); buffers such as acetate, citrate, or phosphate; and tonicity agents such as sodium chloride or dextrose. The pH can be adjusted with acids or bases. Such preparations may be enclosed in ampoules, disposable syringes, or multiple dose vials.

Solutions or suspensions used for intravenous administration include a carrier such as physiological saline, bacteriostatic water, Cremophor EL™ (BASF, Parsippany, N.J.), ethanol, or polyol. In all cases, the composition must be sterile and fluid for easy syringability. Proper fluidity can often be obtained using lecithin or surfactants. The composition must also be stable under the conditions of manufacture and storage. Prevention of microorganisms can be achieved with antibacterial and antifungal agents, e.g., parabens, chlorobutanol, phenol, ascorbic acid, thimerosal, etc. In many cases, isotonic agents (sugar), polyalcohols (mannitol and sorbitol), or sodium chloride may be included in the composition. Prolonged absorption of the composition can be accomplished by adding an agent which delays absorption, e.g., aluminum monostearate and gelatin.

Oral compositions include an inert diluent or edible carrier. The composition can be enclosed in gelatin or compressed into tablets. For the purpose of oral administration, the antibodies can be incorporated with excipients and placed in tablets, troches, or capsules. Pharmaceutically compatible binding agents or adjuvant materials can be included in the composition. The tablets, troches, and capsules, may contain (1) a binder such as microcrystalline cellulose, gum tragacanth or gelatin; (2) an excipient such as starch or lactose, (3) a disintegrating agent such as alginic acid,

Primogel, or corn starch; (4) a lubricant such as magnesium stearate; (5) a glidant such as colloidal silicon dioxide; or (6) a sweetening agent or a flavoring agent.

The pharmaceutical composition may also be administered by a transmucosal or transdermal route. For example, antibodies that comprise a Fc portion may be capable of crossing mucous membranes in the intestine, mouth, or lungs (via Fc receptors). Transmucosal administration can be accomplished through the use of lozenges, nasal sprays, inhalers, or suppositories. Transdermal administration can also be accomplished through the use of a composition containing ointments, salves, gels, or creams known in the art. For transmucosal or transdermal administration, penetrants appropriate to the barrier to be permeated are used. For administration by inhalation, the antibodies are delivered in an aerosol spray from a pressured container or dispenser, which contains a propellant (e.g., liquid or gas) or a nebulizer.

In certain embodiments, the pharmaceutical compositions are prepared with carriers to protect the active component against rapid elimination from the body. Biodegradable polymers (e.g., ethylene vinyl acetate, polyanhydrides, polyglycolic acid, collagen, polyorthoesters, polylactic acid) are often used. Methods for the preparation of such formulations are known by those skilled in the art. Liposomal suspensions can be used as pharmaceutically acceptable carriers too. The liposomes can be prepared according to established methods known in the art (U.S. Pat. No. 4,522,811).

The pharmaceutical compositions are administered in therapeutically effective amounts as described. Therapeutically effective amounts may vary with the subject's age, condition, sex, and severity of medical condition. Appropriate dosage may be determined by a physician based on clinical indications. The compositions may be given as a bolus dose to maximize the circulating levels of active component of the composition for the greatest length of time. Continuous infusion may also be used after the bolus dose.

As used herein, the term “subject” is intended to include human and non-human animals. The term “non-human animals” of the invention includes all vertebrates, such as non-human primates, sheep, dogs, cows, chickens, amphibians, reptiles, etc.

Examples of dosage ranges that can be administered to a subject can be chosen from: 1 μg/kg to 20 mg/kg, 1 μg/kg to 10 mg/kg, 1 μg/kg to 1 mg/kg, 10 μg/kg to 1 mg/kg, 10 μg/kg to 100 μg/kg, 100 μg/kg to 1 mg/kg, 250 μg/kg to 2 mg/kg, 250 μg/kg to 1 mg/kg, 500 μg/kg to 2 mg/kg, 500 μg/kg to 1 mg/kg, 1 mg/kg to 2 mg/kg, 1 mg/kg to 5 mg/kg, 5 mg/kg to 10 mg/kg, 10 mg/kg to 20 mg/kg, 15 mg/kg to 20 mg/kg, 10 mg/kg to 25 mg/kg, 15 mg/kg to 25 mg/kg, 20 mg/kg to 25 mg/kg, and 20 mg/kg to 30 mg/kg (or higher). These dosages may be administered daily, weekly, biweekly, monthly, or less frequently, for example, biannually, depending on dosage, method of administration, disorder or symptom(s) to be treated, and individual subject characteristics. Dosages can also be administered via continuous infusion (such as through a pump). The administered dose may also depend on the route of administration. For example, subcutaneous administration may require a higher dosage than intravenous administration.

In certain circumstances, it may be advantageous to formulate compositions in dosage unit form for ease of administration and uniformity of dosage. Dosage unit form as used herein refers to physically discrete units suited for the patient. Each dosage unit contains a predetermined quantity of antibody calculated to produce a therapeutic effect in association with the carrier. The dosage unit depends on the characteristics of the antibodies and the particular therapeutic effect to be achieved.

Toxicity and therapeutic efficacy of the pharmaceutical composition can be determined by standard pharmaceutical procedures in cell cultures or experimental animals, e.g., determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used to formulate a dosage range in humans. The dosage of these compounds may lie within the range of circulating antibody concentrations in the blood, which includes an ED₅₀ with little or no toxicity. The dosage may vary within this range depending upon the dosage composition form employed and the route of administration. The therapeutically effective dose can be estimated initially using cell culture assays. A dose may be formulated in animal models to achieve a circulating plasma concentration range that includes the IC₅₀ (i.e., the concentration of agent which achieves a half-maximal inhibition of symptoms). The effects of any particular dosage can be monitored by a suitable bioassay. Examples of suitable bioassays include DNA replication assays, transcription-based assays, receptor-binding assays, and other immunological assays.

The antagonists, antibodies and binding fragments discussed above may also be used to detect the presence of at least one of IL-22, IL-17A, IL-17F, IL-1F6, IL-1F8, IL-1F9, and IL-1Rrp2 in a biological sample. These cytokines and receptors can be detected either extracellularly or intracellularly using methods known in the art, including the methods disclosed in this application. By correlating the presence or level of these proteins with a medical condition, one of skill in the art can diagnose the associated medical condition. For example, IL-22 induces changes associated with those caused by inflammatory cytokines (such as IL-1 and TNFα), and inhibitors of IL-22 ameliorate symptoms in an animal model of rheumatoid arthritis (WO 02/068476 A2).

Antibody-based detection methods are well known in the art, and include ELISA, radioimmunoassays, immunoblots, Western blots, flow cytometry, immunofluorescence, immunoprecipitation, and other related techniques. The antibodies may be provided in a diagnostic kit. The kit may contain other components, packaging, instructions, or other material to aid the detection of the protein and use of the kit.

Antibodies may be modified with detectable markers, including ligand groups (e.g., biotin), fluorophores and chromophores, radioisotopes, electron-dense reagents, or enzymes. Enzymes are detected by their activity. For example, horseradish peroxidase is detected by its ability to convert tetramethylbenzidine (TMB) to a blue pigment, quantifiable with a spectrophotometer. Other suitable binding partners include biotin and avidin, IgG and protein A, and other receptor-ligand pairs known in the art.

Antibodies can also be functionally linked (e.g., by chemical coupling, genetic fusion, non-covalent association or otherwise) to at least one other molecular entity, such as another antibody (e.g., a bispecific or a multispecific antibody), toxins, radioisotopes, cytotoxic or cytostatic agents, among others. Other permutations and possibilities are apparent to those of ordinary skill in the art, and they are considered equivalents within the scope of this invention.

In certain embodiments, when the detection method is an in vitro method, it includes: (1) contacting the sample or a control sample with a first reagent that binds to a first target (for example and not limitation IL-1F6), and a second reagent that binds to a second target (for example, and not limitation IL-1F8), and (2) detecting formation of a complex between the first and second reagents and the sample or the control sample, wherein a statistically significant change in the formation of the complex in the sample relative to a control sample, is indicative of the presence of the cytokines in the sample. In one embodiment, the method includes contacting a sample comprising cells with a labeled reagent, such as a fluorescent antibody, that binds to a target selected from one of IL-22, IL-1F6, IL-1F8, IL-1F9, IL-1Rrp2, or IL-17A within the cells. The amount of reagent detected within a cell is directly proportional to the amount of intracellular target expressed within the cell. In certain embodiments, the sample is a blood sample from a patient. Additional samples in which expression levels can be measured include, but are not limited to, synovial fluid, buccal swabs, skin, material removed for biopsy, for example and not limitation, skin of a psoriasis patient removed for a biopsy, semen, hair, bone, urine, nasal secretions, and sputum, including, but not limited to, fluids obtained from a bronchial lavage during a bronchoscopy.

The detection method can also be an in vivo detection method (e.g., in vivo imaging in a subject). The method can be used to diagnose a disorder, e.g., a disorder as described herein. The method includes: (1) administering a first reagent that binds to a first target (for example and not limitation IL-1F6) and a second reagent that binds to a second target (for example, and not limitation IL-1F8) to a subject or a control subject under conditions that allow binding of the first and second reagents to their targets, and (2) detecting formation of a complex between the first and second reagents and their targets, wherein a statistically significant change in the formation of the complex in the subject relative to a control, e.g., a control subject, is indicative of the presence of the cytokines

In certain embodiments, a detection method can also be an in vitro detection method that measures mRNA levels. The method can be used to diagnose a disorder, e.g., a disorder as described herein. In certain embodiments, the method includes: (1) harvesting a sample from a patient, and (2) detecting the levels of target mRNA in the sample. In certain embodiments, the target mRNA includes at least one of IL-22 mRNA, IL-1F6 mRNA, IL-1F8 mRNA, IL-1F9 mRNA, IL-1Rrp2 mRNA and IL-17A mRNA. Methods for detecting and quantitating mRNA are known in the art. In certain embodiments, the method comprises quantitative RT-PCR. In certain embodiments, the sample is a blood sample from a patient. Additional samples in which expression levels can be measured include, but are not limited to, synovial fluid, buccal swabs, skin, material removed for biopsy, for example and not limitation, skin of a psoriasis patient removed for a biopsy, semen, hair, bone, urine, nasal secretions, and sputum, including, but not limited to, fluids obtained from a bronchial lavage during a bronchoscopy.

### EXAMPLES

### Example 1

### Increased Gene and Protein Expression of IL-1 Cytokines in a Psoriatic-Like Mouse Model

Psoriasis vulgaris is a chronic inflammatory skin disease characterized by hyperproliferative epidermis and mixed cutaneous lymphocytic infiltrate. While initially regarded as a primary disease of keratinocyte alteration, effective immune-modulating therapies demonstrate the role played by the immune cells and the cytokines that they produce in psoriatic disease pathogenesis.

4×10⁵ of CD4⁺ CD45RB^{hi}CD25⁻ cells were transferred into pathogen-free CB17 scid/scid to induce scaly and raised skin plaques with certain characteristics resembling human psoriasis. At day 60 post transfer, the majority of mice developed psoriasis-like skin inflammation, such as thickening of the epidermis (acanthosis) due to increased proliferation of keratinocytes (parakeratosis), downward papillary projections of the epidermis (basilar papilla), as well as inflammatory cell infiltrates in the epidermis and dermis.

To test cytokine components of skin lesions, mouse ears were harvested at day 70 post adaptive transfer and subjected to quantitative RT-PCR to evaluate the gene expression of IL-1F6, IL-1F8, IL-1F9 and their specific receptor IL-1Rrp2. RNA was isolated from frozen mouse ear biopsies using the QIAGEN RNeasy® kit (QIAGEN). The expression of cytokine genes was examined using Taqman® RT-PCR kits with pre-qualified primers and probes (Applied Biosystems) according to the following table.

TABLE 2

| | Applied Biosystems | Applied Biosystems |
|---|---|---|
| | Catalog Number | Catalog Number |
| Gene | Human | Mouse |
| Il-1Rrp2 | Hs00187259_m1 | Mm00519250_m1 |
| IL-1F6 | Hs00205367_m1 | Mm00457645_m1 |
| IL-1F8 | Hs00205359_m1 | Mm01337545_m1 |
| IL-1F9 | Hs00219742_m1 | Mm00463327_m1 |
| GAPDH | Hs99999905_m1 | Mm99999915_g1 |

Gene expression was normalized to the expression of the housekeeping gene GAPDH with the assumption of 1,000 copies of GAPDH mRNA per cell.

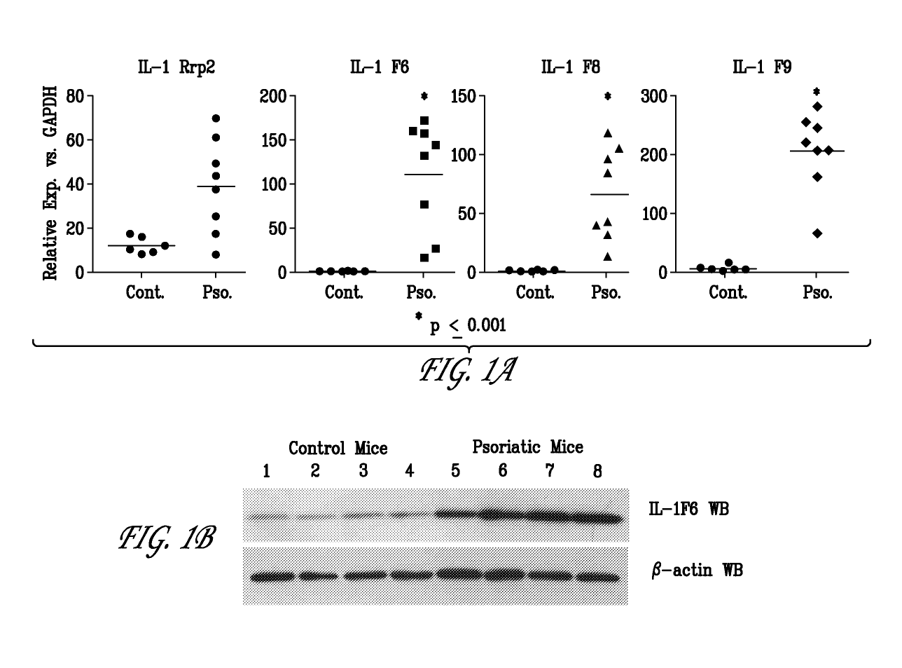

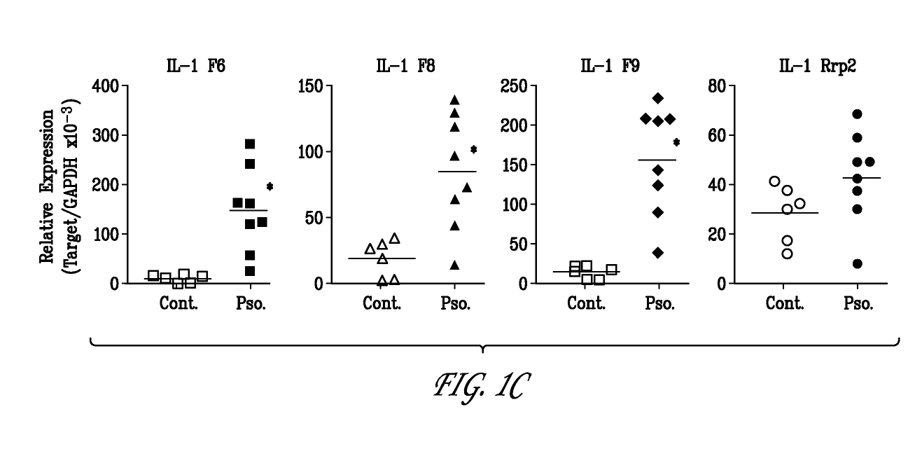

Compared to the control mice that received saline injection, mice that developed psoriatic-like formation had significantly elevated expression of IL-1 cytokines, IL-1F6 (-500 fold), IL-1F8 (-60 fold), and IL-1F9 (-50 fold) in the ear samples (FIGS. 1a and 1c). The transcripts of their receptor, IL-1Rrp2 were also increased about 3-5 fold, although the increase did not reach statistical significance. In addition, a three fold increase in the expression of IL-1F6 and a five fold increase in the expression of IL-1F9 were detected in the white blood cells of the mice with psoriasis.

To confirm that the increased gene expression was correlated with increased protein production in the cells, the protein level of one of the three cytokines, IL-1F6, was checked in the ear lysate by western blot. Three to four times more IL-1F6 protein was detected by western blot in the ear biopsies from recipients of CD4⁺ CD45RB^{hi}CD25⁻ cells compared to control ear samples (FIG. 1b), indicating increased protein production consistent with the observed increased gene expression.

### Example 2

### IL-22 Regulates the Expression of IL-1 Cytokines in the Mouse Skin

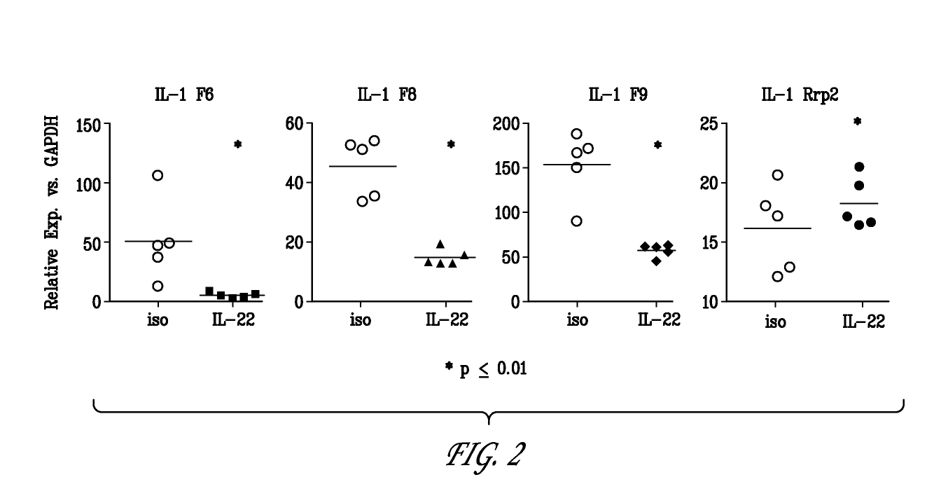

IL-22 is required for Th17 cell mediated pathology in psoriasis and IL-22 neutralization alone is sufficient to prevent psoriasis progression. 4×10⁵ of CD4⁺ CD45RB^{hi}CD25⁻ cells were transferred into pathogen-free CB17 scid/scid mice as described in Example 1. An antibody that neutralizes IL-22 was intravenously injected into the pathogen-free CB17 scid/scid mice once a week starting at the day of the adaptive transfer of CD4⁺ CD45RB^{hi}CD25⁻ T cells. Intravenous injection of the antibody that neutralizes IL-22 prevented the development of the psoriasis-like lesions in the recipient mice. Correlated with the diminished symptoms on day 70 post transfer, the transcript levels of IL-1 cytokines in the ears were also reduced: ˜9-fold reduction in IL-1F6 expression level, ˜2.25-fold reduction in IL-1F8 expression level and ˜2.2-fold reduction in IL-1F9 expression level (FIG. 2). However, IL-22 neutralization had no effect on the elevated expression level of the receptor IL-1 Rrp2 gene. Transcript levels were measured by RT-PCR as described in Example 1.

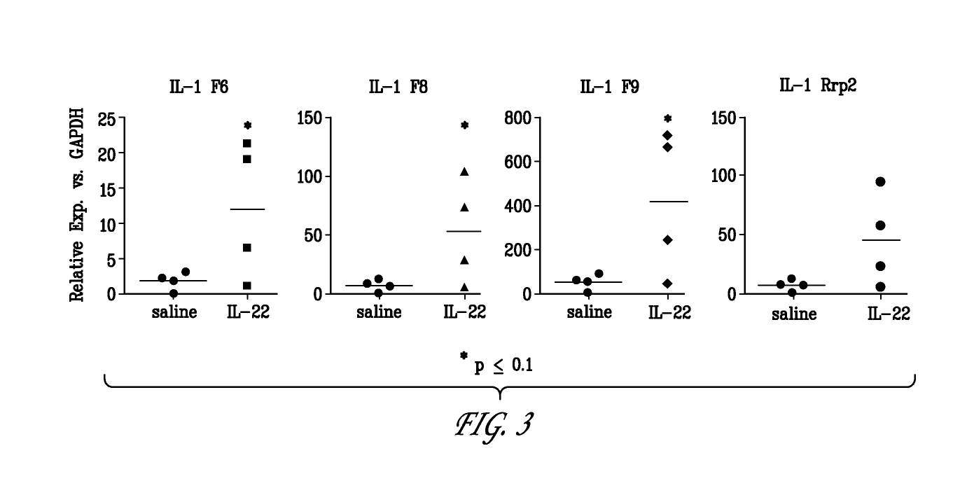

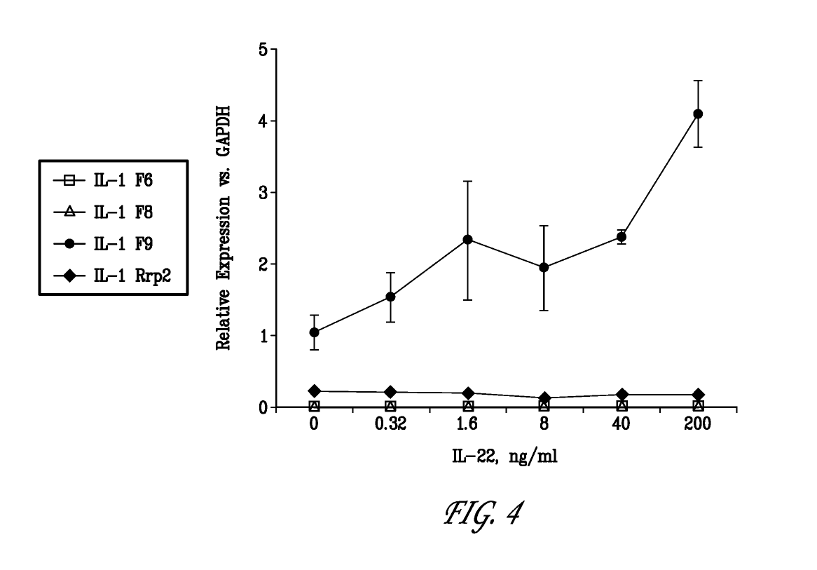

To demonstrate that the expression of IL-1 cytokines was related to the concentration of IL-22 at the local level, wild type BALB/c mice were injected directly into the ears with 500 ng of recombinant mouse IL-22 every other day for two weeks. Six hours after the last treatment, mouse ears were harvested, and transcript levels were measured by RT-PCR as described in Example 1. Transcript levels for IL-1F6, IL-1F8 and IL-1F9 all increased ˜6-fold in the right ears that received IL-22 compared to in the left ears of the same mice that received saline as controls (FIG. 3). Receptor IL-1Rrp2 mRNA also had a trend of up-regulation in the IL-22 treated ears. These data suggest that the Th17 cytokine, IL-22, can directly regulate the gene expression of IL-1 cytokines at the site of inflammation.

### Example 3

### Cytokine Effects on the Production of IL-1 Cytokines in the Human Keratinocytes

IL-22, IL-17A, and IL-17F

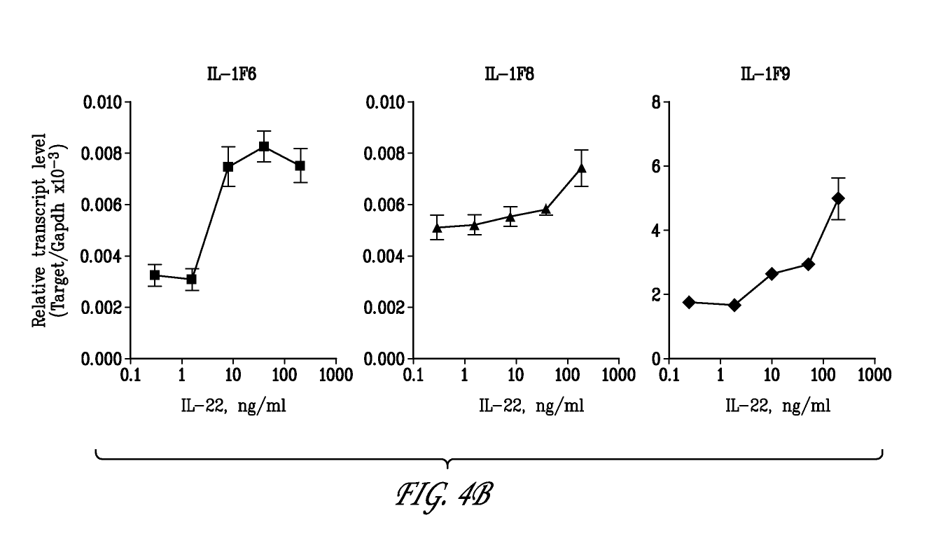

To further confirm the direct induction of IL-1 isoforms by IL-22, primary human epithelial keratinocytes were treated with IL-22 for two days before examining gene expression by quantitative RT-PCR. Human keratinocytes were used as pl-p3 passages after thaw and were treated with variable concentrations of IL-22 from 0 ng/ml to 200 ng/ml as shown in FIG. 4a or 4b. Transcript levels were measured by RT-PCR as described in Example 1. In a first set of experiments, in keratinocytes treated with 200 ng/ml IL-22, IL-1F9 transcript levels increased 4-fold compared to non-treated cells. IL-1F8 transcripts were up-regulated from non-detectable levels to detectable levels. IL-1F6 transcripts were under detectable level and IL-1Rrp2 expression had no change. In a second set of experiments, a dose dependent increase in IL-1F6, IL-1F8, and IL-1F9 transcripts was observed. Despite a vast difference in transcript copy numbers (relative to gapdh, E⁻⁶ for illf6, E⁻⁵ for illf8 and E⁻³ for illf9 transcripts), a 2-4-fold increase in illf6, illf8 and illf9 transcript levels was generally detected in keratinocytes cultured with 200 ng/ml of IL-22 compared with those cultured with media alone. (FIG. 4b) However, IL-22 had no effect on the receptor 0112 expression (data not shown).

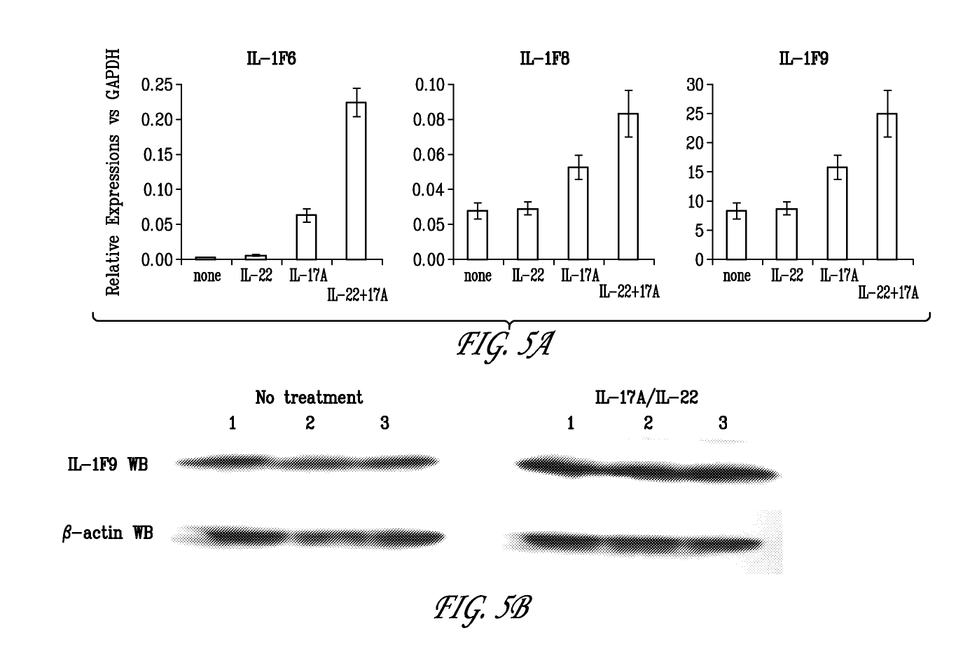

IL-22 and IL-17A are co-expressed by Th17 cells. IL-22 and IL-17A act synergistically to enhance the expression of several antimicrobial peptides, e.g., β-defensin 2, S100A7, S100A8 and S100A9. To further investigate the functional relationship of these Th17 cytokines, the expression of IL-1 isoforms and their receptor was examined in primary keratinocytes treated with combinations of IL-22, IL-17A, and IL-17F. As shown in FIG. 5a, primary keratinocytes were treated with IL-22 (200 ng/ml) or IL-17A(20 ng/ml) alone or the combination of IL-22 (200 ng/ml) and IL-17A (20ng/m1) for two days before examination of gene expression. Transcript levels were measured by RT-PCR as described in Example 1. Although IL-17A alone can induce the expression of IL-1F6 (-20-fold) , IL-1F8 (-1-fold), and IL-1F9 (-7-fold), the presence of IL-22 synergistically induced the expression of IL-1F6 (-80-fold) and IL-1F9 (-15-fold), and additively enhanced the expression of IL-1F8 (-2-fold) (FIGS. 5a and 5c). However, treatment with IL-17F alone did not induce the gene expression of these IL-1 cytokines and their receptor IL-1Rrp2, and the combination of IL-22 and IL-17F did not induce the gene expression of the IL-1 cytokines and IL-1 Rrp2 more than what was observed with 11-22 alone. The amount of IL-1F9 protein in IL-22+IL-17A treated keratinocytes was examined by western blot (FIG. 5b), confirming that the increased transcript level was correlated with increased protein production. At least a two-fold increase of signal was detected by western blot in the IL-22+IL-17A treated keratinocytes compared to those without treatment.

TNF-α

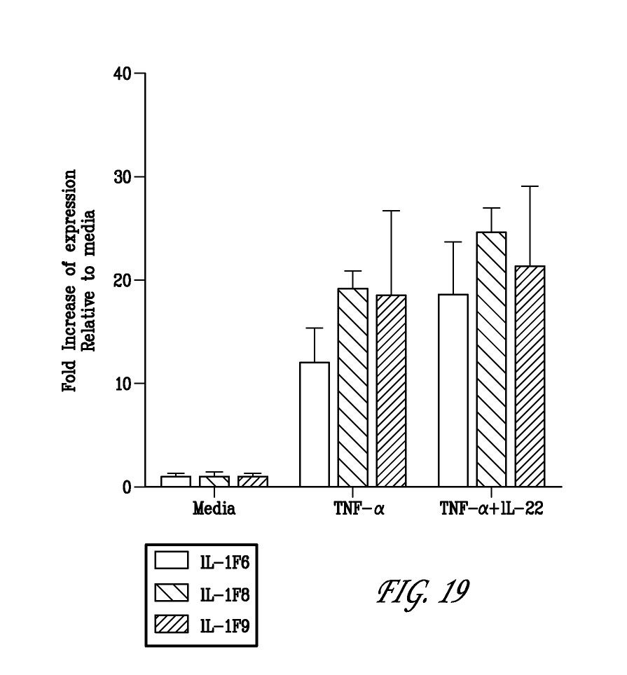

TNF-α is another important pro-inflammatory cytokine that initiates and maintains inflammatory responses in the skin. Clinical studies have demonstrated that blocking the TNF pathway is an effective treatment in psoriasis patients. Gottlieb A. B. et al., J. Immunol. 175, 2721-29 (2005). In light of the clinical efficacy of TNF blockade, the possible regulation of IL-1F6, IL-1F8, and IL-1F9 by TNF-α was examined in our in vitro human keratinocyte culture system. As shown in FIG. 19, TNF-α stimulated keratinocytes had a 10-20-fold increase in illf6, illf8, and illf9 gene transcripts compared to cells cultured with media alone. The increased IL-1 gene expression could be further enhanced with addition of IL-22 to the culture. Together, these data suggest that IL-1F6, IL-1F8 and IL-1F9 are effector cytokines downstream of their induction by IL-17A, IL-22 and TNF-α in the skin.

IFN-γ and IL-12

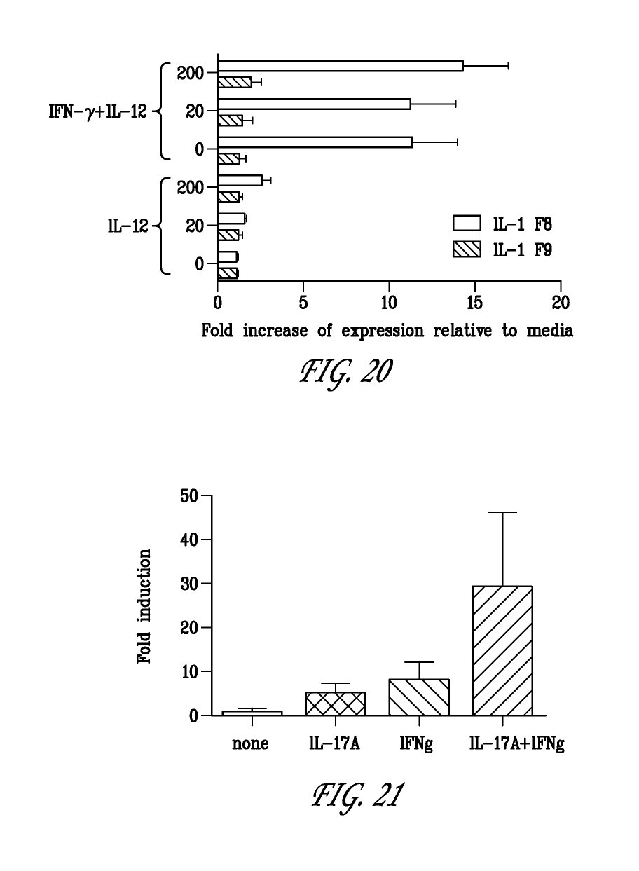

The induction and progression of tissue damage in psoriasis has been traditionally linked to Th1 T cells and their signature cytokines IFN-γ and IL-12. The effect of these Thl cytokines on expression of illf6, i11f8, and illf9 genes was examined in primary keratinocytes. As shown in FIG. 20, expression of illf8 was induced 2 fold by IL-12 at 200 ng/ml concentration and transcript levels of illf9 showed no change. Treatment with IFN-γ alone induced about a 10-fold increase of illf8 transcript while having minimal effect on illf9 gene expression. Addition of IL-12 to the IFN-γ keratinocyte cultures did not substantially enhance i11f8 or illf9 transcripts induced by IFN-γ alone. Of note, in contrast to the significant increase in illf6 transcript levels in keratinocytes in response to Th17 cytokines or TNF-α, Th1 cytokines had no effect on the expression of illf6. These data demonstrate that in our in vitro keratinocyte system, the expression of IL-1F6, IL-1F8, and IL-1F9 is predominantly regulated by Th17 cytokines but not by Th1 cytokines.

IL-17A and IFN-γ

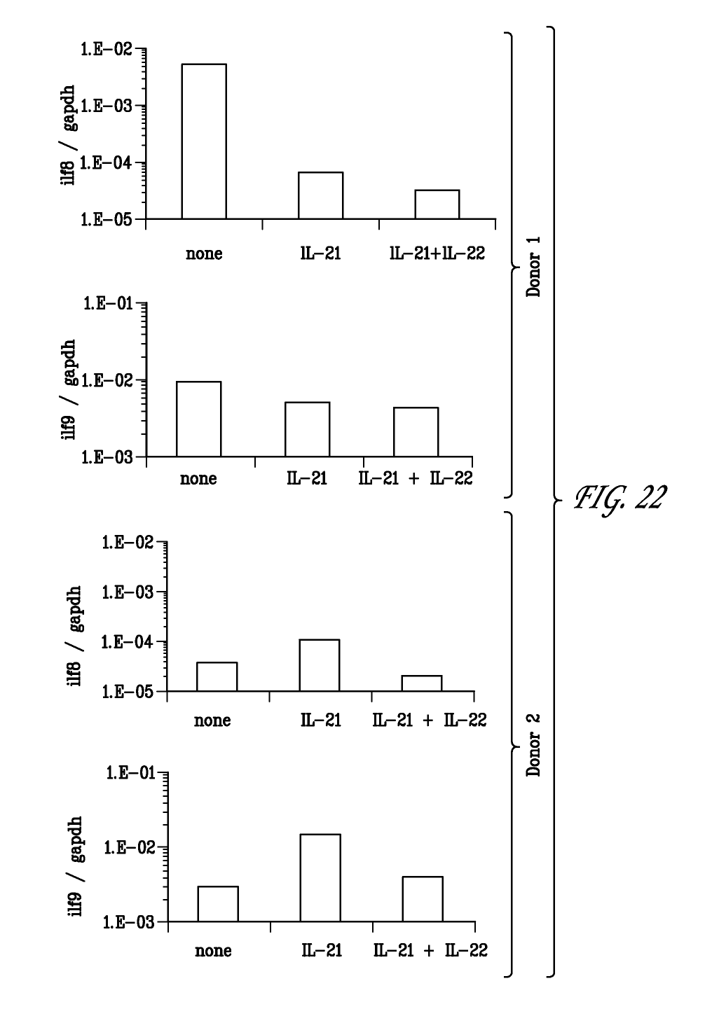

Because Th1 cell and Th17 cells are often co-localized in human psoriatic plaques, the combined effect of IL-17A and IFN-γ on the expression of IL-1 isoforms by human keratinocytes was examined. As shown in FIG. 21, IL-17A enhanced the induction of the illf8 transcript by IFN-y by another 3-fold. However, this cytokine combination does not enhance the increase of illf6 or illf9 transcript by IL-17A (data not shown).

### Example 4

### Increased IL-1 Cytokine Gene Expression Correlated with IL-17A, IL-22, TNF-α, and IFN-γ in the Human Psoriasis Skin Lesions

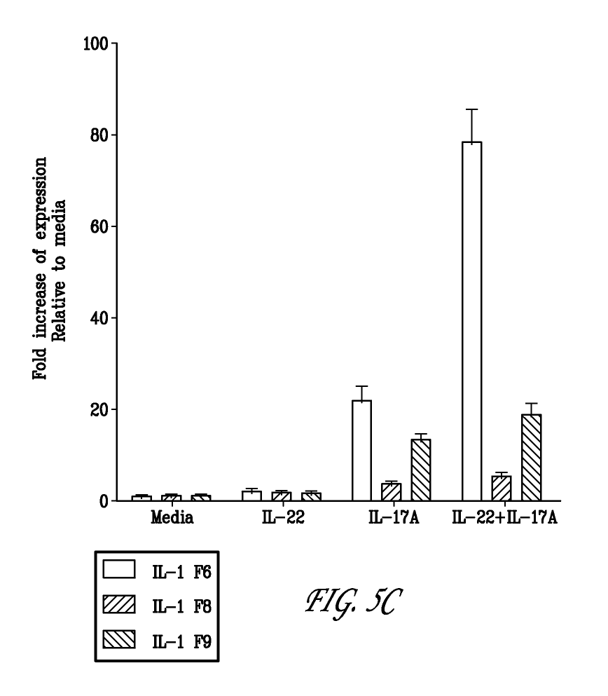

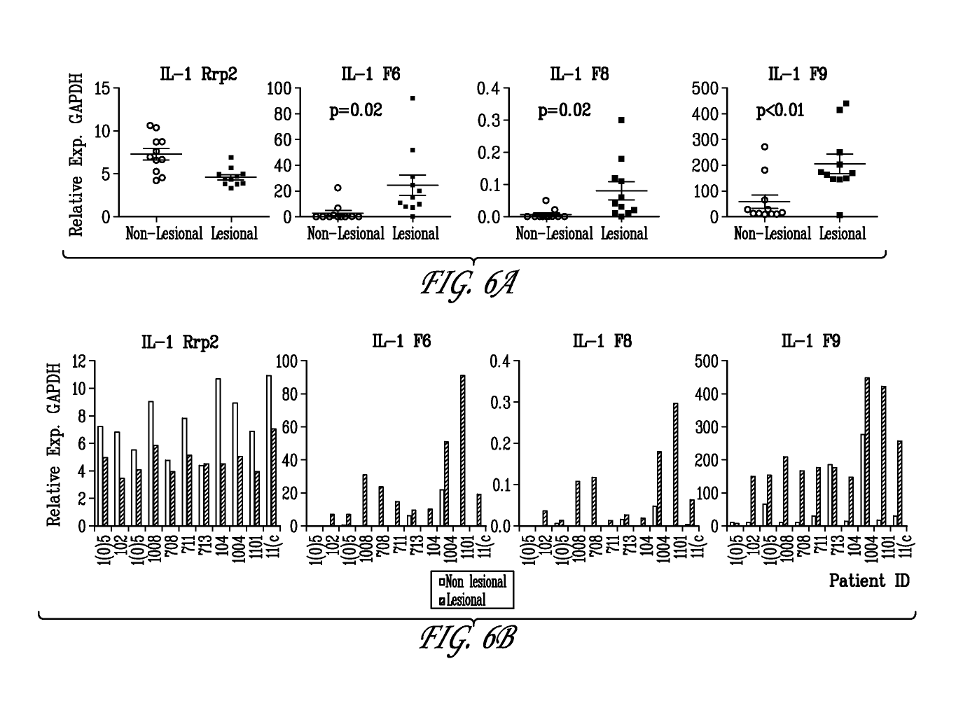

To confirm the observations in the mouse model of psoriasis, human paired non-lesional and lesional skin samples that were obtained from psoriatic patients were examined. Expression levels of a panel of pro-inflammatory cytokines in 11 paired skin samples were examined using quantitative RT-PCR. Transcript levels were measured by RT-PCR as described in Example 1. All patients had increased expression of IL-1F6 (average of about 20-fold higher), IL-1F8 (average of 100-fold higher), and IL-1F9 (average of 4-fold higher) in the psoriatic lesions either as a group (FIG. 6a) or individually (FIGS. 6b and 6c). IL-1F9 mRNA reached the highest copy number per cell: 44% of GAPDH mRNA copies (FIG. 6a), indicating a strong biological function in skin inflammation. Elevated IL-1Rrp2 transcripts in the psoriatic lesions were not detected. The expression level of IL-1Rrp2 was down-modulated in the lesions compared with the mRNA copies in the normal skin tissues.

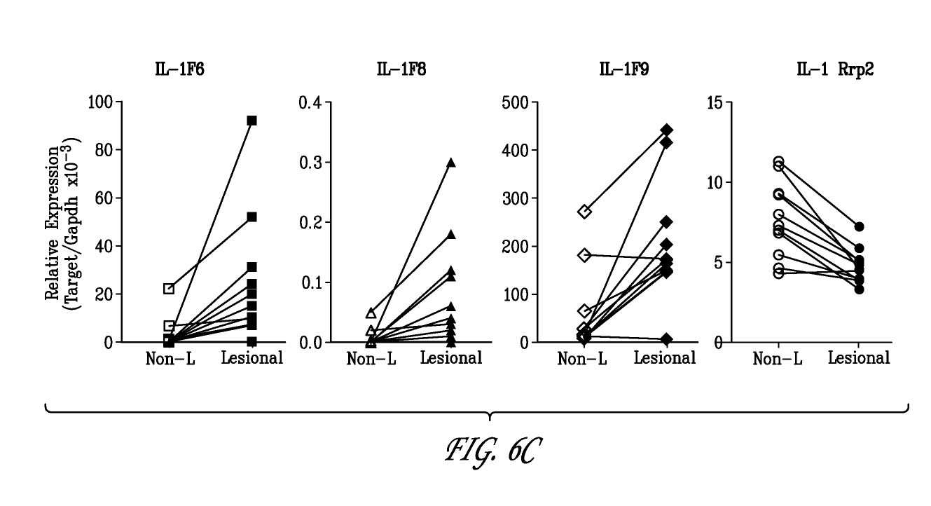

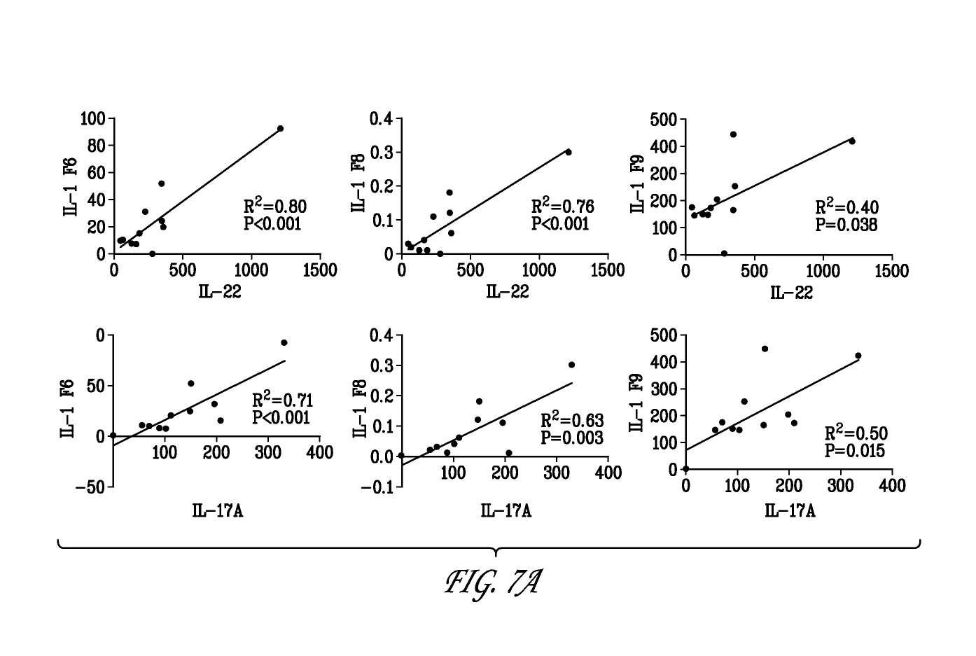

As expected, the expression of all three IL-1 isoforms in human skin lesions strongly correlated with the expression of Th17 cytokines IL-22, IL-21, and IL-17A (Table 3, FIG. 7a, and FIG. 8), yet the expression of the receptor IL1RL2 had no correlation with Th17 cytokines (Table 3 and FIG. 7b). Expression of IL-21R is also correlated with the IL-1 isoforms, but not with IL-22R or IL-22BP. Consistent with the above observation of the effect of IL-17F on keratinocytes in vitro, there was no correlation between the expression of IL-1 isoforms and the expression of IL-17F (FIG. 8). Additionally, expression of the three IL-1 isoforms was compared to expression of IL-22R, IL-22BP, IL-21, IL-21R, IL-23, and TGFα. The expression of IL-1F6 and IL-1F8 also correlated with the other Th17 cytokine, IL-21, and its receptor. The expression of IL-1F9 also correlated with the expression of IL-23, and the expression of IL-1F6, IL-1F8, and IL-1F9 correlated with the expression of TGFα.

TABLE 3: Correlation of cytokine expression profiles in human psoriatic lesions. In Table 3, statistical correlations were determined by Pearson correlation test and p values were determined by two-tail Student's t test.

| | | F6 | F8 | F9 | RL2 |
|---|---|---|---|---|---|
| IL-12p35 | r | −0.324 | −0.324 | −0.524 | 0.154 |
| | p | 0.331 | 0.332 | 0.098 | 0.651 |
| | R² | 0.105 | 0.105 | 0.274 | 0.024 |
| IL-17A | r | 0.848 | 0.795 | 0.705 | −0.037 |
| | p | 0.001 | 0.004 | 0.015 | 0.913 |
| | R² | 0.719 | 0.632 | 0.497 | 0.001 |
| IL-17F | r | 0.491 | 0.562 | 0.237 | −0.351 |
| | p | 0.125 | 0.072 | 0.482 | 0.290 |
| | R² | 0.241 | 0.316 | 0.056 | 0.123 |
| IL-21 | r | 0.823 | 0.781 | 0.535 | −0.169 |
| | p | 0.002 | 0.005 | 0.090 | 0.619 |
| | R² | 0.678 | 0.609 | 0.286 | 0.029 |
| IL-21R | r | 0.932 | 0.919 | 0.726 | −0.303 |
| | p | <0.0001 | <0.0001 | 0.011 | 0.366 |
| | R² | 0.869 | 0.844 | 0.527 | 0.092 |
| IL-22 | r | 0.894 | 0.873 | 0.629 | −0.103 |
| | p | 0.000 | 0.001 | 0.038 | 0.764 |
| | R² | 0.799 | 0.761 | 0.395 | 0.011 |
| IL-22R | r | −0.219 | −0.236 | −0.263 | 0.136 |
| | p | 0.517 | 0.486 | 0.435 | 0.691 |
| | R² | 0.048 | 0.055 | 0.069 | 0.018 |
| IL-22BP | r | −0.481 | −0.507 | −0.572 | 0.499 |
| | p | 0.135 | 0.111 | 0.066 | 0.118 |
| | R² | 0.231 | 0.258 | 0.327 | 0.249 |
| IL-23 | r | 0.588 | 0.600 | 0.790 | 0.000 |
| | p | 0.057 | 0.051 | 0.004 | 1.000 |
| | R² | 0.346 | 0.360 | 0.624 | 0.000 |
| IFN-γ | r | 0.863 | 0.810 | 0.862 | −0.171 |
| | p | 0.001 | 0.003 | 0.001 | 0.615 |
| | R² | 0.745 | 0.656 | 0.742 | 0.029 |
| TNF-α | r | 0.618 | 0.677 | 0.676 | 0.162 |
| | p | 0.043 | 0.022 | 0.023 | 0.635 |
| | R² | 0.382 | 0.459 | 0.457 | 0.026 |

Corroborating the keratinocyte expression of IL-1 isoforms upon TNF-α stimulation in vitro, the expression of TNF correlated with that of IL-1 cytokines in psoriatic lesions. In addition, elevated expression of IFN-γ, but not IL-12p35, was also detected in the lesions and correlated well with the expression of IL-1F6, IL-1F8, and IL-1F9, indicating a collaborative involvement of both Thl and Th17 cells in psoriasis.

### Example 5

### Differentiated Expression of IL-1 Cytokines As Biomarkers in the Animal Models of Autoimmune Diseases

To evaluate the potential of IL-1F6, IL-1F8, IL-1F9 and their receptor as biomarkers of autoimmune diseases, the expression of those genes was examined in blood obtained from three different autoimmune mouse models.

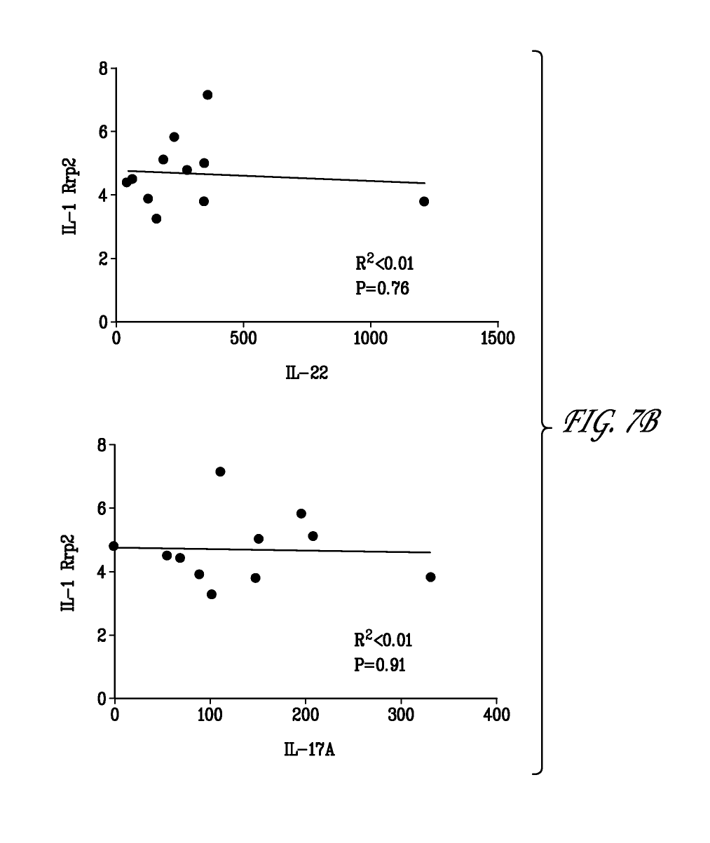

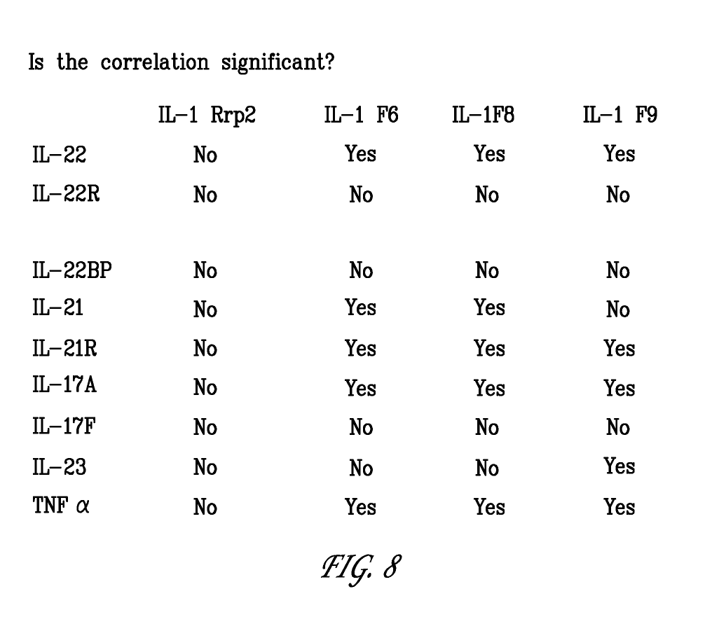

IL-1 isoforms IL-1F6, IL-1F8, and IL-1F9 were examined in the Collagen Induced Arthritis (CIA) mouse model, in which inflammation is induced by immunization with collagen and adjuvant. To induce disease, DBA1 mice were immunized with 200 ng of bovine type II collagen (Chondrex) emulsified in CFA intradermally. On day 21, all mice received a boost of 200 ng of collagen in IFA. On day 35, the blood was collected and immediately subjected to RNA extraction using QIAGEN RNeasy® blood mini kit. White blood cells were used for gene expression analysis via quantitative RT-PCR. Transcript levels were measured by RT-PCR as described in Example 1. Compared to naïve control mice, the mRNA of IL-1F8 and IL-1F9 was increased ˜10-fold and ˜4.3-fold respectively in the blood of diseased mice, while message of IL-1F6 was un-detectable in both diseased and control mice (FIG. 9).

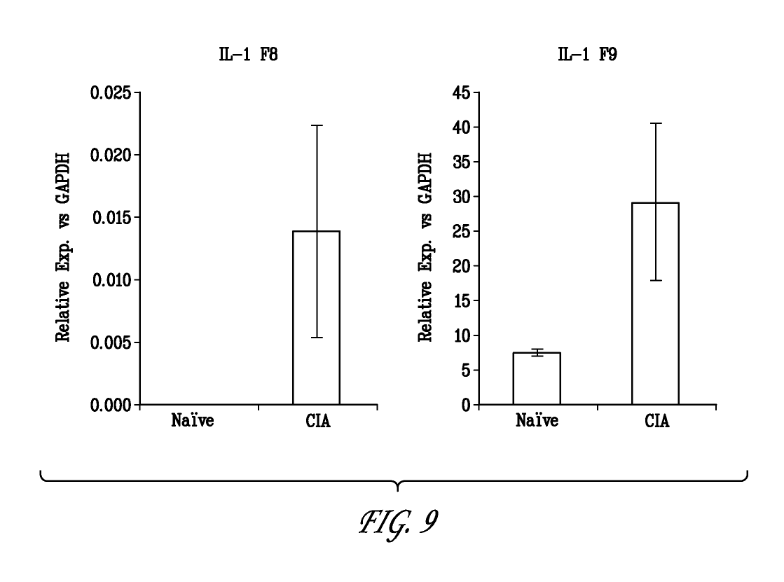

IL-1 isoforms IL-1F6, IL-1F8, and IL-1F9 were also examined in a psoriasis-like skin inflammation mouse model that was induced in scid/scid mice by intravenous transfer of effector (CD4⁺ CD45RB^{hi}CD25⁻) T cells from naïve wide type Balb/c mice. Mouse blood was collected on day 70 of disease induction and gene expression in white blood cells was analyzed. Transcript levels were measured by RT-PCR as described in Example 1. Transcript levels of IL-1F6 and IL-1F9 were increased 3-6 fold in the recipient mice that developed psoriasis as compared to control mice that received saline injection. The transcript level of IL-1F8 in the blood was under the detection level. The expression of the receptor IL-1Rrp2 was also very low and seemed down-regulated in the blood of psoriatic-like mice (FIG. 10).

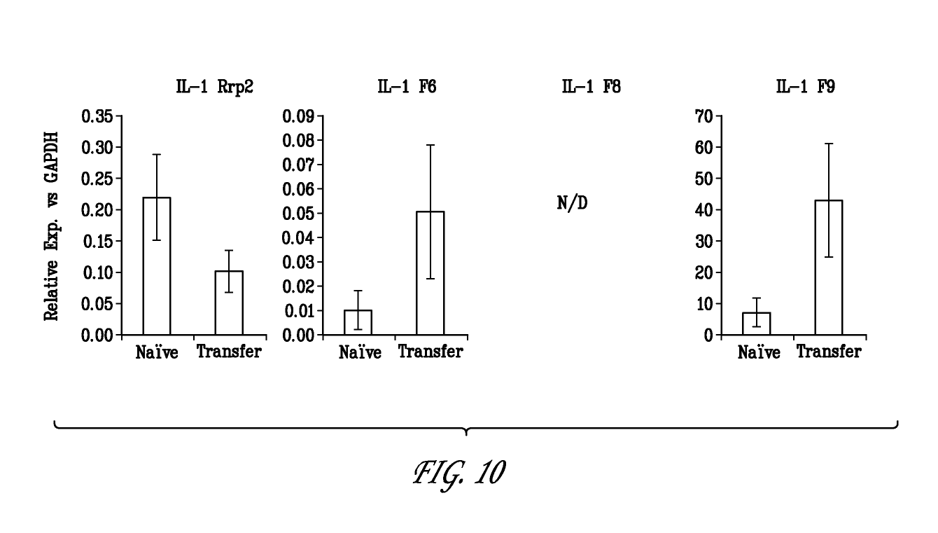

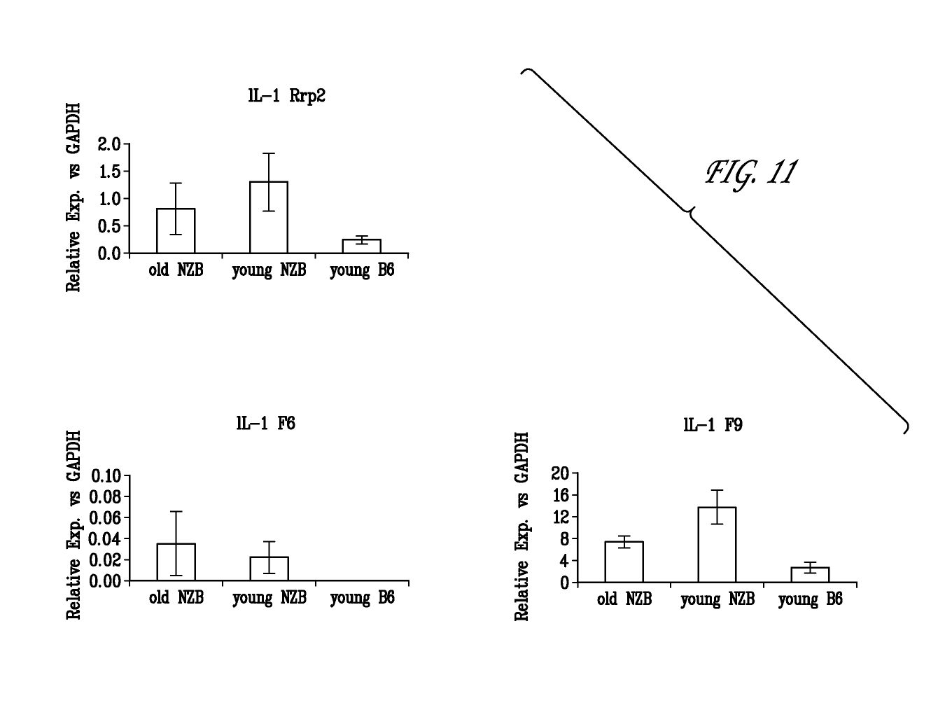

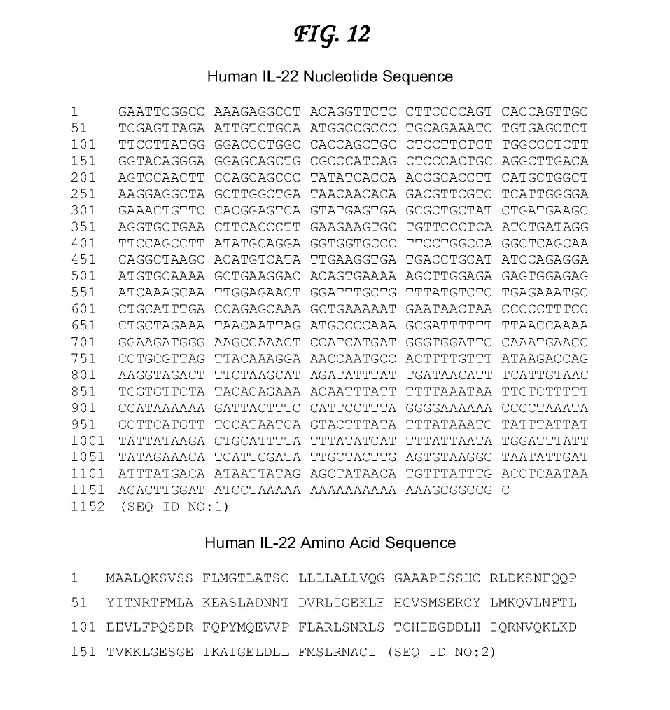

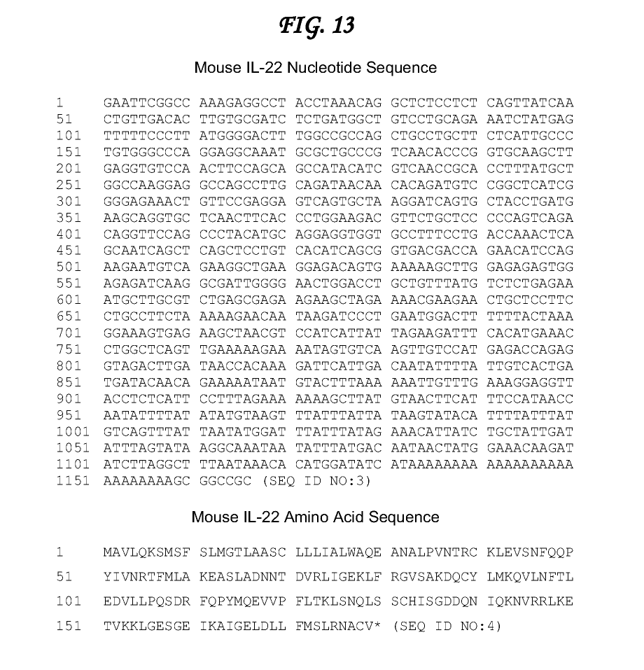

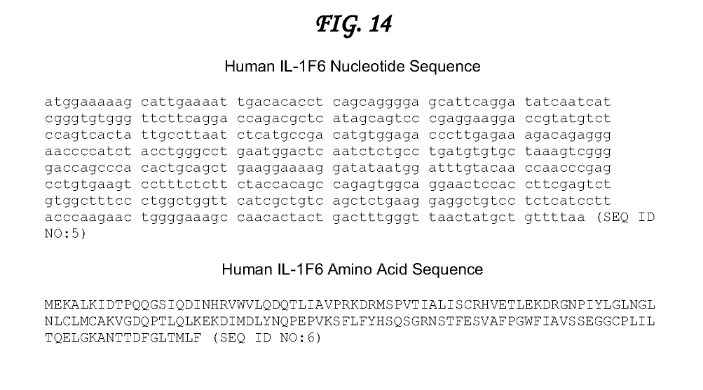

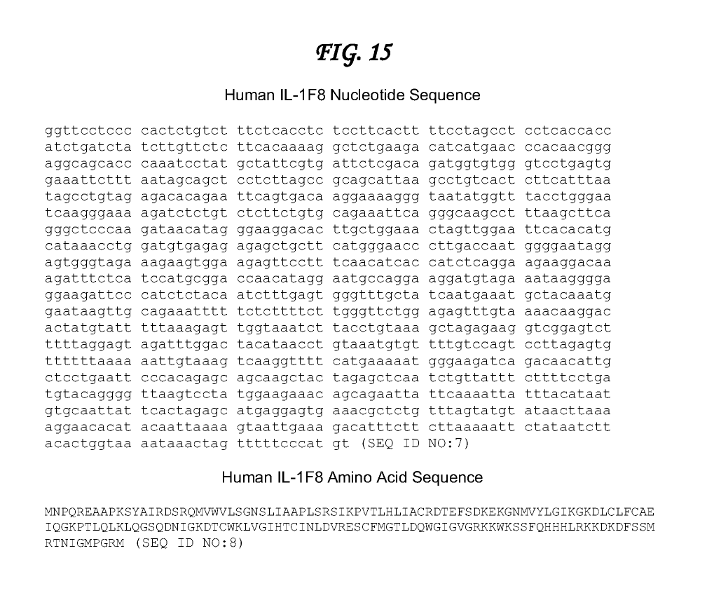

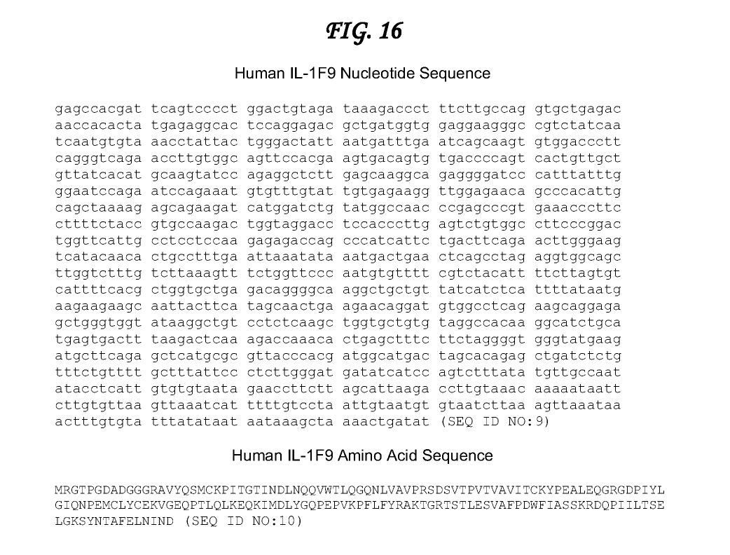

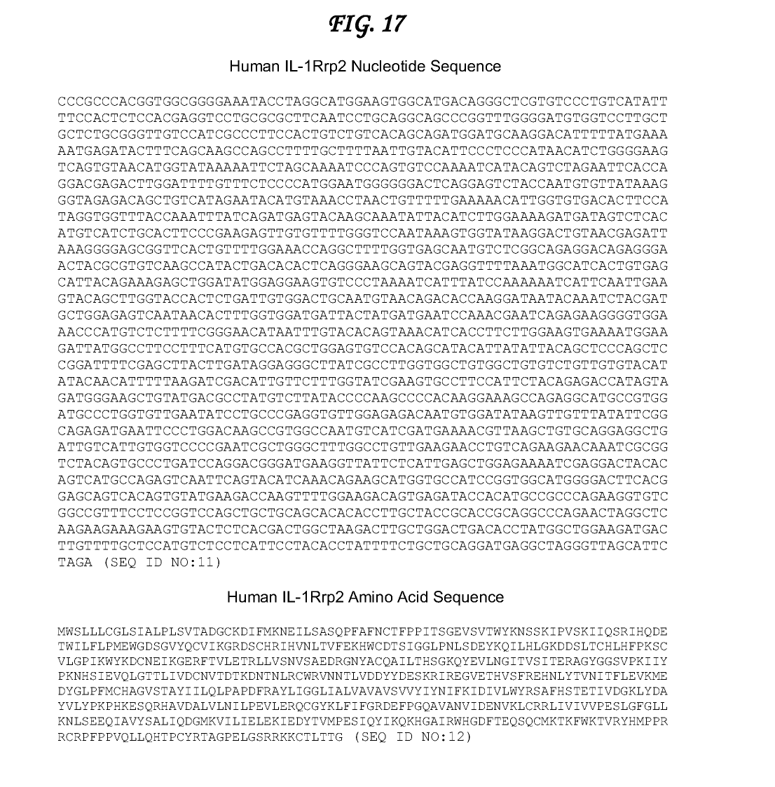

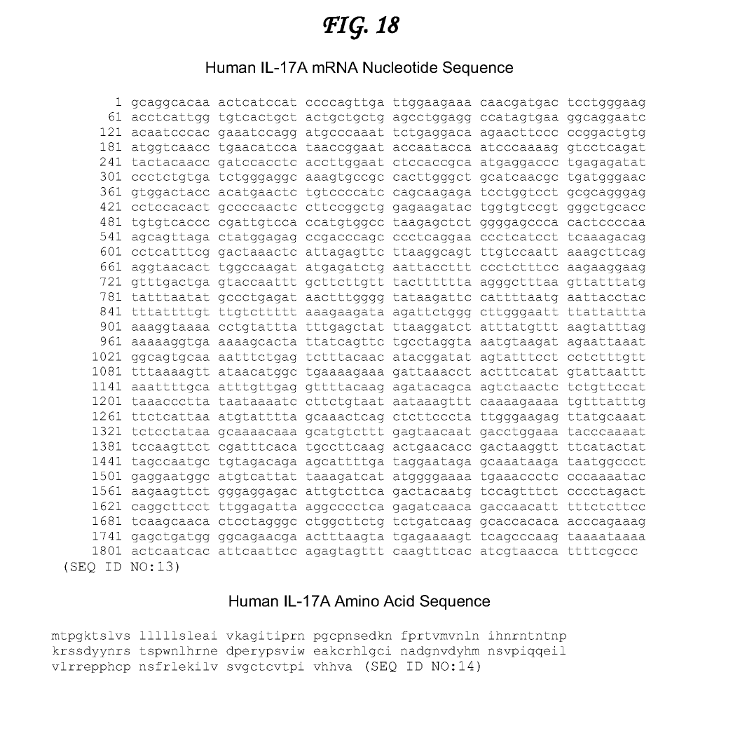

IL-1 isoforms IL-1F6, IL-1F8, and IL-1F9 were also examined in a spontaneous lupus mouse model. The NZBWF/1 strain of mice is genetically susceptible to spontaneous development of lupus. The colony used typically displays detectable lupus symptoms such as proteinuria or anti-dsDNA antibodies at approximately 20 weeks. Blood samples of these mice were collected at 10 weeks before and 7 months after the onset of disease, and gene expression was examined and compared with 10-week old healthy C57BL/6 mice, a strain that does not develop spontaneous lupus. Transcript levels were measured by RT-PCR as described in Example 1. At the 10-week early time point in disease, there was increased gene expression of IL-1F6, IL-1F9, and IL-1Rrp2 (FIG. 11), indicating that these genes may be early biomarkers for lupus. The transcripts for IL-1F6 were further elevated in the 7-month old mice which had developed the lupus symptoms of proteinuria and increased anti-dsDNA antibodies. However, the transcripts of IL-1F9 and receptor IL-1Rrp2 were down-regulated at this time point yet still much higher compared to the control C57BL/6 mice. The transcripts of IL-1F8 were too low to be detected in the blood of NZBWF1/J lupus mice.

These results demonstrated that up-regulated mRNA expression of IL-1 isoforms is associated with inflammatory diseases and can be detected from the blood samples of diseased animals. Previously these isoforms had only been detected in skin tissue samples and keratinocytes. The different levels of induction and expression of IL-1F6, IL-1F8, and IL-1F9 as well as their specific receptor in the different disease models indicate that these genes are aberrantly expressed in various autoimmune diseases, suggesting a potential use of IL-1 isoform expression (e.g. mRNA or protein) as biomarkers for diagnosis of human inflammatory and autoimmune diseases.

### Example 6

### IL-1 Isoforms Are Not Regulated By IL-21 In Vitro

As shown in FIG. 22, IL-21 down-regulates the illf8 and i11f9 transcripts in donor 1, but slightly increases their expression in donor 2, indicating that IL-21 does not directly regulate IL-1 isoforms in keratinocytes. Furthermore, adding IL-22 to the cultures does not significantly affect the expression of IL-1 isoforms. The transcript levels of illf6 in keratinocytes co-cultured with IL-21 were below the limit of detection.

### Example 7

### IL-1α and IL-1β Expression is Not Regulated By Th17 or Th1 Cytokines In Vitro

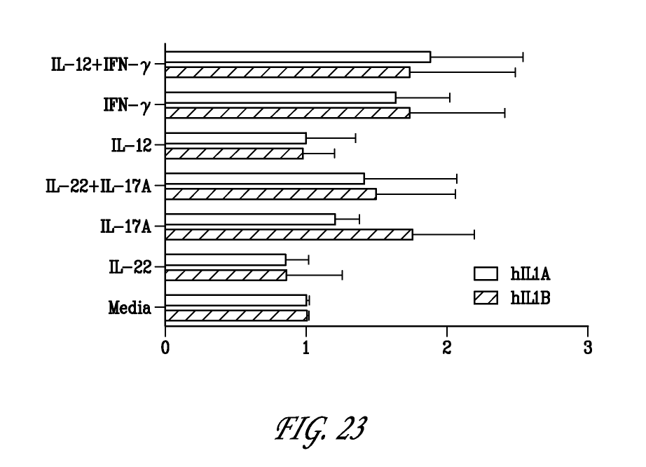

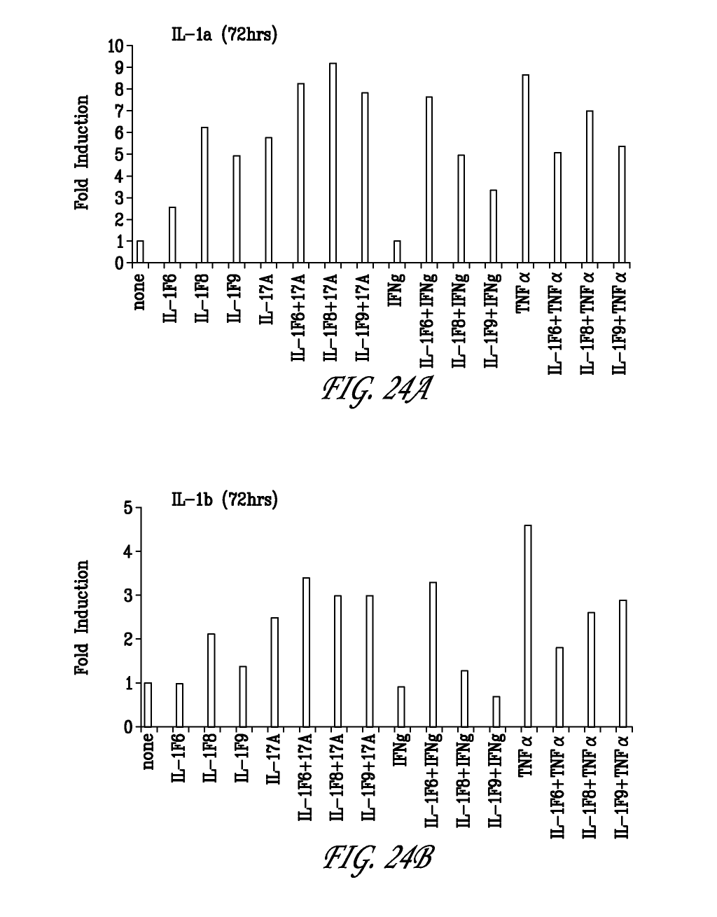

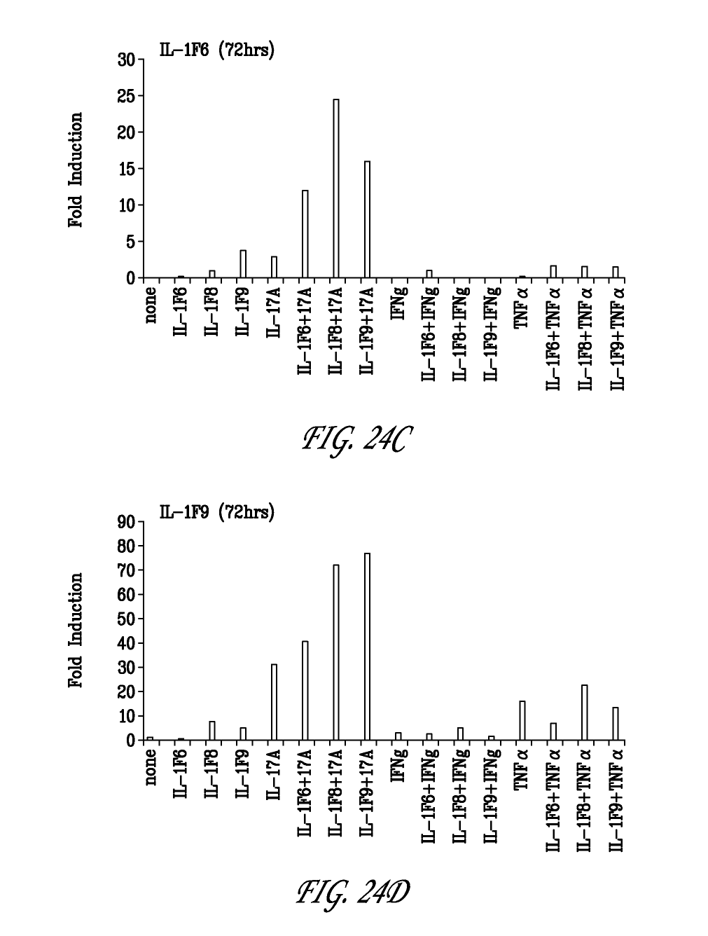

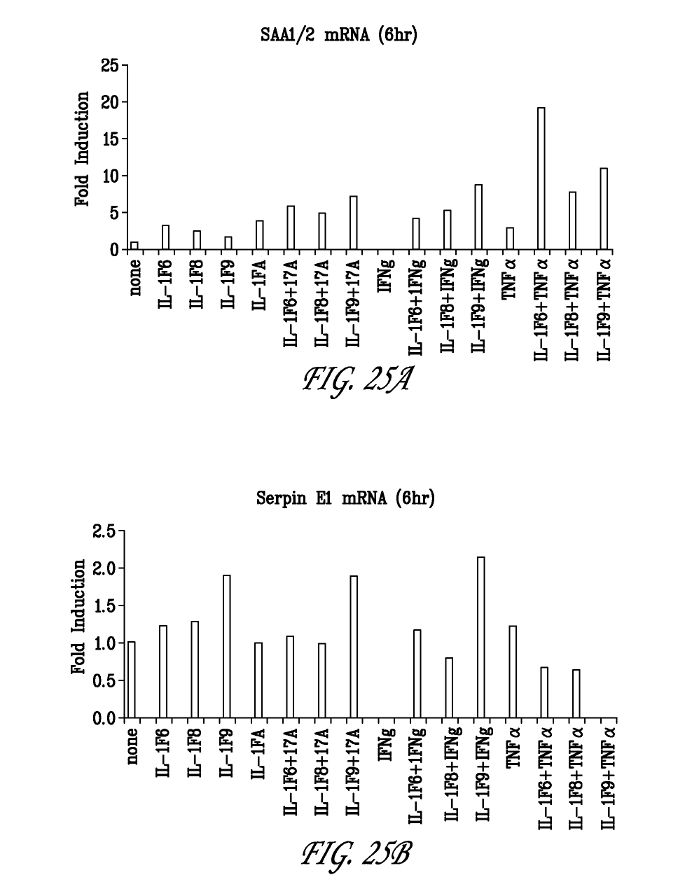

Recent clinical trials have demonstrated that biological agents that block the IL-12/23p40 pathway are efficacious in psoriasis. Krueger, G. et al. N Engl J Med 356, 580-592 (2007). Furthermore, preliminary clinical evidence indicates that blocking IL-17A also has beneficial effect. Patel, D. In ACR/ARHP Annual Scientific Meeting, San Francisco (2008). In contrast, blockade of the IL-1α and IL-1β pathway with a recombinant IL-1R antagonist was shown to be of modest benefit in pilot psoriasis studies. Gibbs, A. G. et al. In 25th European Workshop for Rheumatology Research. Arthritis Research and Therapy, Glasgow, UK. 68 (2005). The regulation of ill a and illb by T cell-derived cytokines was examined in our in vitro culture system. A slight increase (-1.5-2 fold) in the expressions of illa and illb were induced by IL-17A or IFN-γ in keratinocytes (FIG. 23). However, neither IL-22 nor IL-12 had an effect, either alone or in combination with IL-17A or IFN-γ, respectively. These data suggest that IL-1α and IL-1β may not be major local immune mediators that are important for the psoriasis pathogenesis. However, the strong induction of IL-1F6, IL-1F8, and IL-1F9 by Th17 cytokines suggests that these IL-1 isoforms may represent major local mediators of the disease.

### Example 8

### IL-la, IL-1F6, and IL-1F9 Expression Are Regulated By IL-1 Isoforms In Vitro

To investigate the downstream effect of elevated IL-1 isoforms in psoriasis, we examined the ability of by IL-16, IL-1F8, and IL-1F9 to regulate expression of IL-la, IL-1β, IL-1F6, and IL-1F9 in our in vitro culture system (FIG. 24). A slight increase (-2-6 fold) in the expression of illa was induced by IL-1F6, IL-1F8, or IL-1F9 alone. Addition of IL-17A further increased the induction by IL-1 isoforms. However, addition of IFN-y or TNF-a had no further effect on IL-1 a expression. The three IL-1 isoforms showed little regulation of IL-1β expression, either alone or in combination with Th1 or Th17 cytokines All three IL-1 isoforms induced expression of IL-1F6 and this increase was detected as early as 6 hr (data not shown) after co-culture. IL-17A synergized with all three isoforms and strongly enhanced the induction of illf6 mRNA. Addition of TNF-α also enhanced this increase but to a lesser intensity. IL-1F8 and IL-1F9 strongly induced expression of IL-1F9 up to 10 fold. Again, IL-17A synergized with IL-1F8 and IL-1F9 to increase the transcription level of illf9 to ˜80 fold. Addition of TNF-β had slightly additive effects while addition of IFN-γ had no effect. These data suggest that novel IL-1 isoforms not only induce their own gene expressions, but co-operate with Th17 cytokines to further enhance this self-regulation. IFN-y has modest effects on self-enhancement of IL-1 isoforms, which is consistent with its modest effect on the induction of gene expression of IL-1F6, IL-1F8, and IL-1F9.

### Example 9

### Synergistic Effect of IL-1 Isoforms in the Induction of Acute Phase Reactant

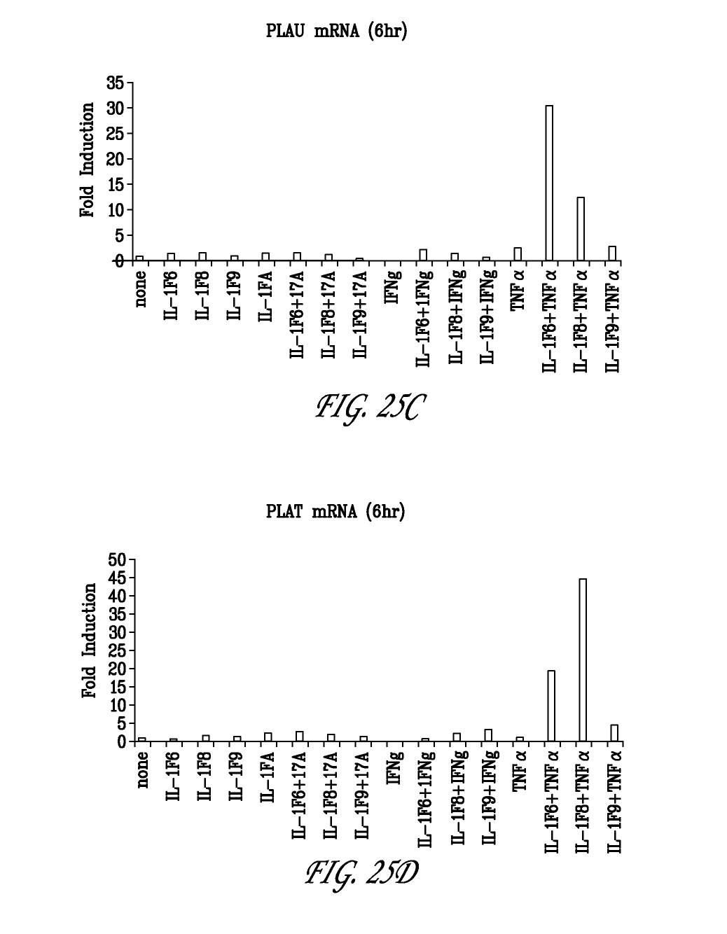

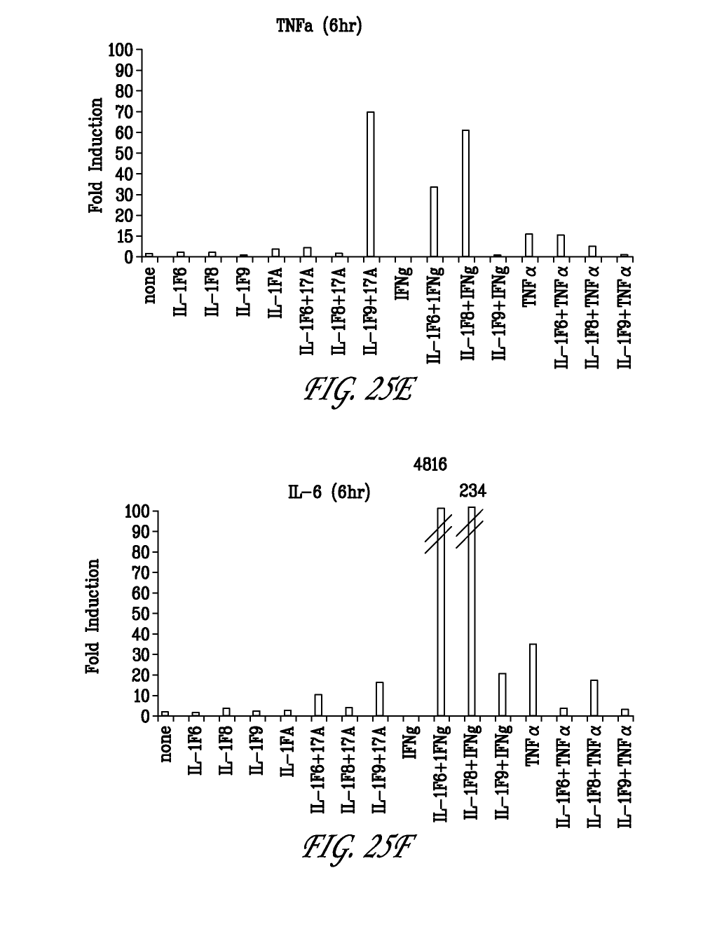

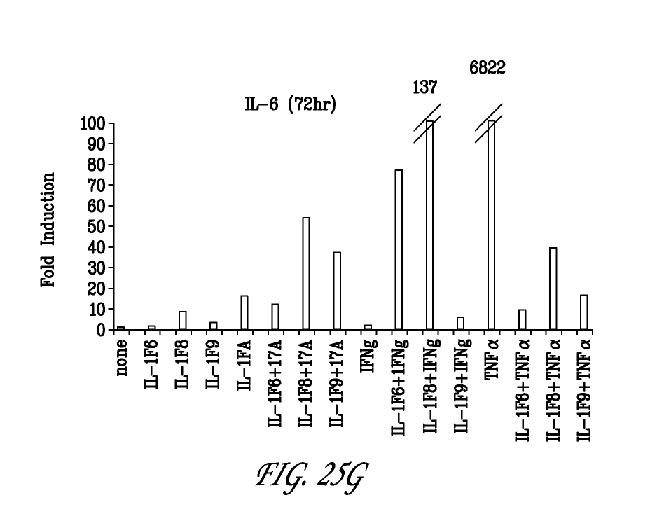

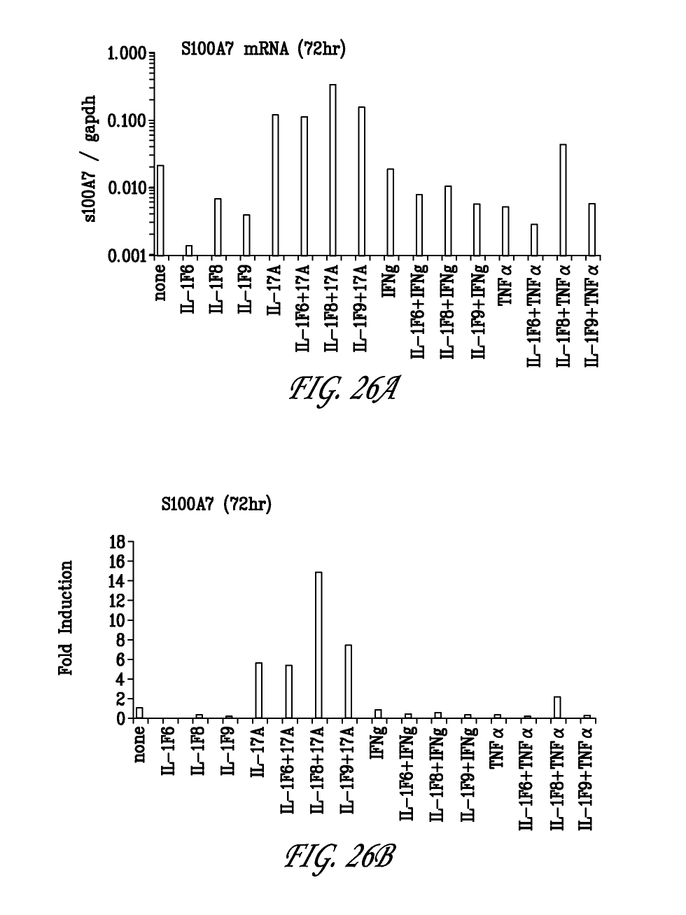

IL-1F6, IL-1F8, or IL-1F9 alone induced a slight increase (˜1-3 fold) in the gene expression of various acute phase reactants, including saa1/2 (serum amyloid A1/2), serpine1 (plasminogen activation inhibitor-1, also known as PAI-1, Serpin E1), plau (urokinase type plasminogen activator, also known as u-PA), plat (tissue plasminogen activator, also known as t-PA), tnfa and il6 (FIG. 25).

Addition of TNF-α had a synergistic effect with all three IL-1 isoforms, strongly enhancing the expression of saa1/2 transcripts induced by the IL-1 isoforms. The addition of IL-17A or IFN-γ had an additive effect on the expression of saa1/2 gene. Addition of TNF-α also had a synergistic effect with IL-1F6 and IL-1F8, strongly enhancing the expression of the plau and plat transcripts induced by each of the IL-1 isoforms. The addition of IL-17A or IFN-g had no further effect on the expression of the plau and plat genes (FIG. 25).

IL-17A synergized with IL-1F9, while IFN-γ synergized with IL-1F6 and IL-1 F8 to increase the expression of tnfa transcripts induced by IL-1 isoforms by ˜40-60 fold. TNF-α induced its own expression by about 10-fold, but no synergy was observed when combined with the IL-1 isoforms (FIG. 25).

IFN-γ synergized with IL-1F6 and IL-1F8 to increase the expression of il6 transcripts by ˜230-4000 fold at 6 hr after co-culture. This strong induction of the il6 gene was still observed at 72 hr post co-culture. IFN-γ also had a synergistic effect with IL-1F9, increasing the expression of il6 transcripts by ˜20 fold. IL-17A also showed synergistic effects with IL-1F6 and IL-1F9 on il6 expression, although the synergistic response was not as strong as that of IFN-γ. Combining TNF-α with the three IL-1 isoforms did not have a synergistic effect on il6 expression (FIG. 25).

### Example 10

### IL-1 Isoforms Induce Antimicrobial Peptides in Cooperation With IL-17A

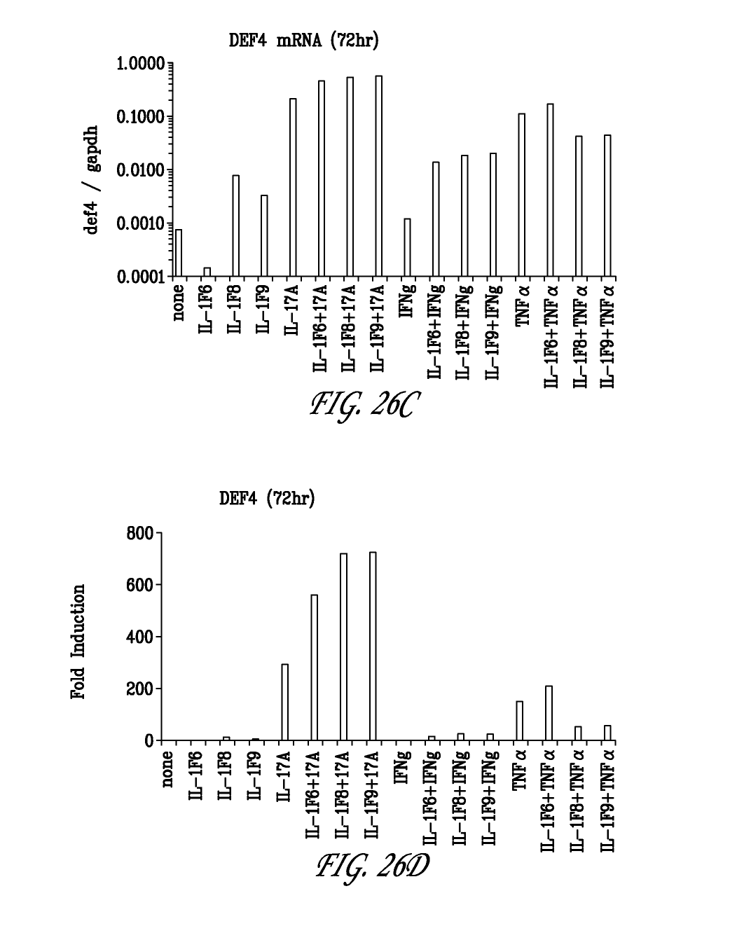

It is known that IL-17A induces the expression of antimicrobial peptides associated with host defense, including β-defensin 2 (gene symbol: def4) and S100A7 (gene symbol: s100a7). To examine whether IL-1 isoforms could induce the same genes by themselves or in combination with Th17 or Th1 cytokines, keratinocytes were incubated with individual IL-1 cytokine or in paired combination of these cytokines IL-1 isoforms did not strongly induce β-defensin 2 or S100A7 gene expression alone, but in combination with IL-17A, IL-1F8 induced ˜16-fold of increase of s100a7 transcripts, while IL-1F6, IL-1F8, and IL-1F9, in combination with IL-17A, increased the level of def4 transcription by ˜600-800 fold. FIG. 26. TNF-a has additive effects with IL-1F8 in the induction of s100a7 expression and IFN-y has additive effects with all three of IL-1 isoforms in the gene expression of def4 (FIG. 26).

The following documents provide additional information on IL-1 cytokines and the IL-1Rrp2 receptor, and are incorporated herein by reference for any purpose.

Berglof, E., R. Andre, B. R. Renshaw, S. M. Allan, C. B. Lawrence, N. J. Rothwell, and E. Pinteaux. 2003. IL-1Rrp2 expression and IL-1F9 (IL-1H1) actions in brain cells. J Neuroimmunol 139:36-43.

Blumberg, H., H. Dinh, E. S. Trueblood, J. Pretorius, D. Kugler, N. Weng, S. T. Kanaly, J. E. Towne, C. R. Willis, M. K. Kuechle, J. E. Sims, and J. J. Peschon. 2007. Opposing activities of two novel members of the IL-1 ligand family regulate skin inflammation. J Exp Med 204:2603-2614.

Dunn, E., J. E. Sims, M. J. Nicklin, and L. A. O'Neill. 2001. Annotating genes with potential roles in the immune system: six new members of the IL-1 family. Trends Immunol 22:533-536.

Kumar, S., P. C. McDonnell, R. Lehr, L. Tierney, M. N. Tzimas, D. E. Griswold, E. A. Capper, R. Tal-Singer, G. I. Wells, M. L. Doyle, and P. R. Young. 2000. Identification and initial characterization of four novel members of the interleukin-1 family. J Biol Chem 275:10308-10314.

Magne, D., G. Palmer, J. L. Barton, F. Mezin, D. Talabot-Ayer, S. Bas, T. Duffy, M. Noger, P. A. Guerne, M. J. Nicklin, and C. Gabay. 2006. The new IL-1 family member IL-1F8 stimulates production of inflammatory mediators by synovial fibroblasts and articular chondrocytes. Arthritis Res Ther 8:R80.

Sims, J., J. Towne, and H. Blumberg. 2006. 11 IL-1 family members in inflammatory skin disease. Ernst Schering Res Found Workshop:187-191.

Sims, J. E. 2002. IL-1 and IL-18 receptors, and their extended family. Curr Opin Immunol 14:117-122.

Sims, J. E., M. J. Nicklin, J. F. Bazan, J. L. Barton, S. J. Busfield, J. E. Ford, R. A. Kastelein, S. Kumar, H. Lin, J. J. Mulero, J. Pan, Y. Pan, D. E. Smith, and P. R. Young. 2001. A new nomenclature for IL-1-family genes. Trends Immunol 22:536-537.

Smith, D. E., B. R. Renshaw, R. R. Ketchem, M. Kubin, K. E. Garka, and J. E. Sims. 2000. Four new members expand the interleukin-1 superfamily. J Biol Chem 275:1169-1175.

Towne, J. E., K. E. Garka, B. R. Renshaw, G. D. Virca, and J. E. Sims. 2004. Interleukin (IL)-1F6, IL-1F8, and IL-1F9 signal through IL-1Rrp2 and IL-1RAcP to activate the pathway leading to NF-kappaB and MAPKs. J Biol Chem 279:13677-13688.

Vos, J. B., M. A. van Sterkenburg, K. F. Rabe, J. Schalkwijk, P. S. Hiemstra, and N. A. Datson. 2005. Transcriptional response of bronchial epithelial cells to Pseudomonas aeruginosa: identification of early mediators of host defense. Physiol Genomics 21:324-336.

Wang, P., B. Meinhardt, R. Andre, B. R. Renshaw, I. Kimber, N. J. Rothwell, and E. Pinteaux. 2005. The interleukin-l-related cytokine IL-1F8 is expressed in glial cells, but fails to induce IL-1 beta signalling responses. Cytokine 29:245-250.

Those skilled in the art will recognize, or be able to ascertain using no more than routine experimentation, many equivalents to the specific embodiments described herein. Such equivalents are encompassed by the following claims.

What is claimed is:

1. A method of detecting an inflammatory disorder comprising identifying upregulation of at least one of (a) at least one isoform of IL-1 and (b) IL-1Rrp2 in a patient, wherein the at least one isoform of IL-1 is IL-1F6, IL-1F8, or IL-1F9.

2. The method of claim 1, wherein the inflammatory disorder is psoriasis, lupus, or arthritis.

3. The method of claim 1, wherein the upregulation of at least one of (a) the at least one isoform of IL-1 and (b) IL-1Rrp2 is determined by detecting mRNA levels.

4. The method of claim 1, wherein the upregulation of at least one of (a) the at least one isoform of IL-1 and (b) IL-1Rrp2 is determined by detecting protein levels.

5. The method of claim 1, wherein the detection of upregulation of at least two of (a) the at least one isoform of IL-1 and (b) IL-1Rrp2 is detected by detecting protein levels of at least one of (a) the at least one isoform of IL-1 and (b) IL-1Rrp2 and by detecting mRNA levels of at least one of (a) the at least one isoform of IL-1 and (b) IL-1Rrp2.

6. A method of treating an IL-22-associated disorder comprising administering at least one inhibitor of at least one of IL-1F6, IL-1F8, and IL-1F9 to a patient with said IL-22-associated disorder.

7. The method of claim 6 wherein the at least one inhibitor is an anti-IL-1F6 antibody.

8. The method of claim 6 wherein the at least one inhibitor is an anti-IL-1F8 antibody.

9. The method of claim 6 wherein the at least one inhibitor is an anti-IL-1F9 antibody.

10. The method of claim 6 wherein the at least one inhibitor is an anti-IL-1Rrp2 antibody.

11. The method of any one of claims 6-10, wherein the IL-22 associated disorder is psoriasis, lupus, or arthritis.

12. A method of treating an inflammatory disorder comprising administering to a patient with an inflammatory disorder a combination of (a) at least one of (i) an anti-IL-1F6 antibody, (ii) an anti-IL-1F8 antibody, (iii) an anti-IL-1F9 antibody, and (iv) an anti-IL-1Rrp2 antibody; and (b) an anti-IL-22 antibody.

13. A method of treating an inflammatory disorder comprising administering to a patient with an inflammatory disorder an anti-IL-1 antibody and an anti-IL-17A antibody.

14. A method of treating an inflammatory disorder comprising administering to a patient with an inflammatory disorder a combination of (a) at least one of (i) an anti-IL-1F6 antibody, (ii) an anti-IL-1F8 antibody, (iii) an anti-IL-1F9 antibody, and (iv) an anti-IL-1Rrp2 antibody; (b) an anti-IL-22 antibody; and (c) an anti-IL-17A antibody.

15. A method for determining the effectiveness of a therapeutic agent in the treatment, reduction, prevention, and/or amelioration of an inflammatory disorder in a subject by detecting a level of gene expression in the subject compared to a level of gene expression in a control sample, wherein the gene expression detected is the gene expression from at least one of IL-1F6, IL-1F8, IL-1F9, IL-1Rrp; and wherein a lower level of gene expression in the subject compared to the control indicates effectiveness of the therapeutic agent in the treatment, reduction, prevention, and/or amelioration of the inflammatory disorder in the subject.

16. The method of any one of claims 12-15, wherein the inflammatory disease is psoriasis, lupus, or arthritis.


SEQUENCE LISTING (14 sequences)

SEQ ID NO: 1 - DNA, 1191 bp, Homo sapiens
gaattcggccaaagaggcctacaggttctccttccccagtcaccagttgctcgagttagaattgtctgcaatggccgccctgcagaaatctgtgagctctttccttatggggaccctggccaccagctgcctccttctcttggccctcttggtacagggaggagcagctgcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcatttgaccagagcaaagctgaaaaatgaataactaaccccctttccctgctagaaataacaattagatgccccaaagcgattttttttaaccaaaaggaagatgggaagccaaactccatcatgatgggtggattccaaatgaacccctgcgttagttacaaaggaaaccaatgccacttttgtttataagaccagaaggtagactttctaagcatagatatttattgataacatttcattgtaactggtgttctatacacagaaaacaatttattttttaaataattgtctttttccataaaaaagattactttccattcctttaggggaaaaaacccctaaatagcttcatgtttccataatcagtactttatatttataaatgtatttattattattataagactgcattttatttatatcattttattaatatggatttatttatagaaacatcattcgatattgctacttgagtgtaaggctaatattgatatttatgacaataattatagagctataacatgtttatttgacctcaataaacacttggatatcctaaaaaaaaaaaaaaaaaagcggccgc

SEQ ID NO: 2 - protein, 179 aa, Homo sapiens
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 3 - DNA, 1166 bp, Mus sp.
gaattcggccaaagaggcctacctaaacaggctctcctctcagttatcaactgttgacacttgtgcgatctctgatggctgtcctgcagaaatctatgagtttttcccttatggggactttggccgccagctgcctgcttctcattgccctgtgggcccaggaggcaaatgcgctgcccgtcaacacccggtgcaagcttgaggtgtccaacttccagcagccatacatcgtcaaccgcacctttatgctggccaaggaggccagccttgcagataacaacacagatgtccggctcatcggggagaaactgttccgaggagtcagtgctaaggatcagtgctacctgatgaagcaggtgctcaacttcaccctggaagacgttctgctcccccagtcagacaggttccagccctacatgcaggaggtggtgcctttcctgaccaaactcagcaatcagctcagctcctgtcacatcagcggtgacgaccagaacatccagaagaatgtcagaaggctgaaggagacagtgaaaaagcttggagagagtggagagatcaaggcgattggggaactggacctgctgtttatgtctctgagaaatgcttgcgtctgagcgagaagaagctagaaaacgaagaactgctccttcctgccttctaaaaagaacaataagatccctgaatggacttttttactaaaggaaagtgagaagctaacgtccatcattattagaagatttcacatgaaacctggctcagttgaaaaagaaaatagtgtcaagttgtccatgagaccagaggtagacttgataaccacaaagattcattgacaatattttattgtcactgatgatacaacagaaaaataatgtactttaaaaaattgtttgaaaggaggttacctctcattcctttagaaaaaaagcttatgtaacttcatttccataaccaatattttatatatgtaagtttatttattataagtatacattttatttatgtcagtttattaatatggatttatttatagaaacattatctgctattgatatttagtataaggcaaataatatttatgacaataactatggaaacaagatatcttaggctttaataaacacatggatatcataaaaaaaaaaaaaaaaaaaaaaaaaagcggccgc

SEQ ID NO: 4 - protein, 179 aa, Mus sp.
MAVLQKSMSFSLMGTLAASCLLLIALWAQEANALPVNTRCKLEVSNFQQPYIVNRTFMLAKEASLADNNTDVRLIGEKLFRGVSAKDQCYLMKQVLNFTLEDVLLPQSDRFQPYMQEVVPFLTKLSNQLSSCHISGDDQNIQKNVRRLKETVKKLGESGEIKAIGELDLLFMSLRNACV

SEQ ID NO: 5 - DNA, 477 bp, Homo sapiens
atggaaaaagcattgaaaattgacacacctcagcaggggagcattcaggatatcaatcatcgggtgtgggttcttcaggaccagacgctcatagcagtcccgaggaaggaccgtatgtctccagtcactattgccttaatctcatgccgacatgtggagacccttgagaaagacagagggaaccccatctacctgggcctgaatggactcaatctctgcctgatgtgtgctaaagtcggggaccagcccacactgcagctgaaggaaaaggatataatggatttgtacaaccaacccgagcctgtgaagtcctttctcttctaccacagccagagtggcaggaactccaccttcgagtctgtggctttccctggctggttcatcgctgtcagctctgaaggaggctgtcctctcatccttacccaagaactggggaaagccaacactactgactttgggttaactatgctgttttaa

SEQ ID NO: 6 - protein, 158 aa, Homo sapiens
MEKALKIDTPQQGSIQDINHRVWVLQDQTLIAVPRKDRMSPVTIALISCRHVETLEKDRGNPIYLGLNGLNLCLMCAKVGDQPTLQLKEKDIMDLYNQPEPVKSFLFYHSQSGRNSTFESVAFPGWFIAVSSEGGCPLILTQELGKANTTDFGLTMLF

SEQ ID NO: 7 - DNA, 1172 bp, Homo sapiens
ggttcctccccactctgtctttctcacctctccttcacttttcctagcctcctcaccaccatctgatctatcttgttctcttcacaaaaggctctgaagacatcatgaacccacaacgggaggcagcacccaaatcctatgctattcgtgattctcgacagatggtgtgggtcctgagtggaaattctttaatagcagctcctcttagccgcagcattaagcctgtcactcttcatttaatagcctgtagagacacagaattcagtgacaaggaaaagggtaatatggtttacctgggaatcaagggaaaagatctctgtctcttctgtgcagaaattcagggcaagcctttaagcttcagggctcccaagataacatagggaaggacacttgctggaaactagttggaattcacacatgcataaacctggatgtgagagagagctgcttcatgggaacccttgaccaatggggaataggagtgggtagaaagaagtggaagagttcctttcaacatcaccatctcaggaagaaggacaaagatttctcatccatgcggaccaacataggaatgccaggaaggatgtagaaataaggggaggaagattcccatctctacaatctttgagtgggtttgctatcaatgaaatgctacaaatggaataagttgcagaaatttttctcttttcttgggttctggagagtttgtaaaacaaggacactatgtatttttaaagagttggtaaatcttacctgtaaagctagagaaggtcggagtctttttaggagtagatttggactacataacctgtaaatgtgttttgtccagtccttagagtgttttttaaaaaattgtaaagtcaaggttttcatgaaaaatgggaagatcagacaacattgctcctgaattcccacagagcagcaagctactagagctcaatctgttatttcttttcctgatgtacaggggttaagtcctatggaagaaacagcagaattattcaaaattatttacataatgtgcaattattcactagagcatgaggagtgaaacgctctgtttagtatgtataacttaaaaggaacacatacaattaaaagtaattgaaagacatttcttcttaaaaattctataatcttacactggtaaaataaactagtttttcccatgt

SEQ ID NO: 8 - protein, 164 aa, Homo sapiens
MNPQREAAPKSYAIRDSRQMVWVLSGNSLIAAPLSRSIKPVTLHLIACRDTEFSDKEKGNMVYLGIKGKDLCLFCAEIQGKPTLQLKLQGSQDNIGKDTCWKLVGIHTCINLDVRESCFMGTLDQWGIGVGRKKWKSSFQHHHLRKKDKDFSSMRTNIGMPGRM

SEQ ID NO: 9 - DNA, 1180 bp, Homo sapiens
gagccacgattcagtcccctggactgtagataaagaccctttcttgccaggtgctgagacaaccacactatgagaggcactccaggagacgctgatggtggaggaagggccgtctatcaatcaatgtgtaaacctattactgggactattaatgatttgaatcagcaagtgtggacccttcagggtcagaaccttgtggcagttccacgaagtgacagtgtgaccccagtcactgttgctgttatcacatgcaagtatccagaggctcttgagcaaggcagaggggatcccatttatttgggaatccagaatccagaaatgtgtttgtattgtgagaaggttggagaacagcccacattgcagctaaaagagcagaagatcatggatctgtatggccaacccgagcccgtgaaacccttccttttctaccgtgccaagactggtaggacctccacccttgagtctgtggccttcccggactggttcattgcctcctccaagagagaccagcccatcattctgacttcagaacttgggaagtcatacaacactgcctttgaattaaatataaatgactgaactcagcctagaggtggcagcttggtctttgtcttaaagtttctggttcccaatgtgttttcgtctacattttcttagtgtcattttcacgctggtgctgagacaggggcaaggctgctgttatcatctcattttataatgaagaagaagcaattacttcatagcaactgaagaacaggatgtggcctcagaagcaggagagctgggtggtataaggctgtcctctcaagctggtgctgtgtaggccacaaggcatctgcatgagtgactttaagactcaaagaccaaacactgagctttcttctaggggtgggtatgaagatgcttcagagctcatgcgcgttacccacgatggcatgactagcacagagctgatctctgtttctgttttgctttattccctcttgggatgatatcatccagtctttatatgttgccaatatacctcattgtgtgtaatagaaccttcttagcattaagaccttgtaaacaaaaataattcttgtgttaagttaaatcatttttgtcctaattgtaatgtgtaatcttaaagttaaataaactttgtgtatttatataataataaagctaaaactgatat

SEQ ID NO: 10 - protein, 169 aa, Homo sapiens
MRGTPGDADGGGRAVYQSMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 11 - DNA, 1964 bp, Homo sapiens
cccgcccacggtggcggggaaatacctaggcatggaagtggcatgacagggctcgtgtccctgtcatattttccactctccacgaggtcctgcgcgcttcaatcctgcaggcagcccggtttggggatgtggtccttgctgctctgcgggttgtccatcgcccttccactgtctgtcacagcagatggatgcaaggacatttttatgaaaaatgagatactttcagcaagccagccttttgcttttaattgtacattccctcccataacatctggggaagtcagtgtaacatggtataaaaattctagcaaaatcccagtgtccaaaatcatacagtctagaattcaccaggacgagacttggattttgtttctccccatggaatggggggactcaggagtctaccaatgtgttataaagggtagagacagctgtcatagaatacatgtaaacctaactgtttttgaaaaacattggtgtgacacttccataggtggtttaccaaatttatcagatgagtacaagcaaatattacatcttggaaaagatgatagtctcacatgtcatctgcacttcccgaagagttgtgttttgggtccaataaagtggtataaggactgtaacgagattaaaggggagcggttcactgttttggaaaccaggcttttggtgagcaatgtctcggcagaggacagagggaactacgcgtgtcaagccatactgacacactcagggaagcagtacgaggttttaaatggcatcactgtgagcattacagaaagagctggatatggaggaagtgtccctaaaatcatttatccaaaaaatcattcaattgaagtacagcttggtaccactctgattgtggactgcaatgtaacagacaccaaggataatacaaatctacgatgctggagagtcaataacactttggtggatgattactatgatgaatccaaacgaatcagagaaggggtggaaacccatgtctcttttcgggaacataatttgtacacagtaaacatcaccttcttggaagtgaaaatggaagattatggccttcctttcatgtgccacgctggagtgtccacagcatacattatattacagctcccagctccggattttcgagcttacttgataggagggcttatcgccttggtggctgtggctgtgtctgttgtgtacatatacaacatttttaagatcgacattgttctttggtatcgaagtgccttccattctacagagaccatagtagatgggaagctgtatgacgcctatgtcttataccccaagccccacaaggaaagccagaggcatgccgtggatgccctggtgttgaatatcctgcccgaggtgttggagagacaatgtggatataagttgtttatattcggcagagatgaattccctggacaagccgtggccaatgtcatcgatgaaaacgttaagctgtgcaggaggctgattgtcattgtggtccccgaatcgctgggctttggcctgttgaagaacctgtcagaagaacaaatcgcggtctacagtgccctgatccaggacgggatgaaggttattctcattgagctggagaaaatcgaggactacacagtcatgccagagtcaattcagtacatcaaacagaagcatggtgccatccggtggcatggggacttcacggagcagtcacagtgtatgaagaccaagttttggaagacagtgagataccacatgccgcccagaaggtgtcggccgtttcctccggtccagctgctgcagcacacaccttgctaccgcaccgcaggcccagaactaggctcaagaagaaagaagtgtactctcacgactggctaagacttgctggactgacacctatggctggaagatgacttgttttgctccatgtctcctcattcctacacctattttctgctgcaggatgaggctagggttagcattctaga

SEQ ID NO: 12 - protein, 575 aa, Homo sapiens
MWSLLLCGLSIALPLSVTADGCKDIFMKNEILSASQPFAFNCTFPPITSGEVSVTWYKNSSKIPVSKIIQSRIHQDETWILFLPMEWGDSGVYQCVIKGRDSCHRIHVNLTVFEKHWCDTSIGGLPNLSDEYKQILHLGKDDSLTCHLHFPKSCVLGPIKWYKDCNEIKGERFTVLETRLLVSNVSAEDRGNYACQAILTHSGKQYEVLNGITVSITERAGYGGSVPKIIYPKNHSIEVQLGTTLIVDCNVTDTKDNTNLRCWRVNNTLVDDYYDESKRIREGVETHVSFREHNLYTVNITFLEVKMEDYGLPFMCHAGVSTAYIILQLPAPDFRAYLIGGLIALVAVAVSVVYIYNIFKIDIVLWYRSAFHSTETIVDGKLYDAYVLYPKPHKESQRHAVDALVLNILPEVLERQCGYKLFIFGRDEFPGQAVANVIDENVKLCRRLIVIVVPESLGFGLLKNLSEEQIAVYSALIQDGMKVILIELEKIEDYTVMPESIQYIKQKHGAIRWHGDFTEQSQCMKTKFWKTVRYHMPPRRCRPFPPVQLLQHTPCYRTAGPELGSRRKKCTLTTG

SEQ ID NO: 13 - DNA, 1859 bp, Homo sapiens
gcaggcacaaactcatccatccccagttgattggaagaaacaacgatgactcctgggaagacctcattggtgtcactgctactgctgctgagcctggaggccatagtgaaggcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccaccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaagagctctggggagcccacactccccaaagcagttagactatggagagccgacccagcccctcaggaaccctcatccttcaaagacagcctcatttcggactaaactcattagagttcttaaggcagtttgtccaattaaagcttcagaggtaacacttggccaagatatgagatctgaattacctttccctctttccaagaaggaaggtttgactgagtaccaatttgcttcttgtttacttttttaagggctttaagttatttatgtatttaatatgccctgagataactttggggtataagattccattttaatgaattacctactttattttgtttgtctttttaaagaagataagattctgggcttgggaattttattatttaaaaggtaaaacctgtatttatttgagctatttaaggatctatttatgtttaagtatttagaaaaaggtgaaaaagcactattatcagttctgcctaggtaaatgtaagatagaattaaatggcagtgcaaaatttctgagtctttacaacatacggatatagtatttcctcctctttgtttttaaaagttataacatggctgaaaagaaagattaaacctactttcatatgtattaatttaaattttgcaatttgttgaggttttacaagagatacagcaagtctaactctctgttccattaaacccttataataaaatccttctgtaataataaagtttcaaaagaaaatgtttatttgttctcattaaatgtattttagcaaactcagctcttccctattgggaagagttatgcaaattctcctataagcaaaacaaagcatgtctttgagtaacaatgacctggaaatacccaaaattccaagttctcgatttcacatgccttcaagactgaacaccgactaaggttttcatactattagccaatgctgtagacagaagcattttgataggaatagagcaaataagataatggccctgaggaatggcatgtcattattaaagatcatatggggaaaatgaaaccctccccaaaatacaagaagttctgggaggagacattgtcttcagactacaatgtccagtttctcccctagactcaggcttcctttggagattaaggcccctcagagatcaacagaccaacatttttctcttcctcaagcaacactcctagggcctggcttctgtctgatcaaggcaccacacaacccagaaaggagctgatggggcagaacgaactttaagtatgagaaaagttcagcccaagtaaaataaaaactcaatcacattcaattccagagtagtttcaagtttcacatcgtaaccattttcgccc

SEQ ID NO: 14 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA